(19) United States
(12) Patent Application Publication
(10) Pub. No.: US 2008/0255073 A1
(43) Pub. Date: Oct. 16, 2008

(54) COMPOUNDS FOR SUSTAINED RELEASE OF ORALLY DELIVERED DRUGS

(75) Inventors: Mark A. GALLOP, Los Altos, CA (US); Kenneth C. Cundy, Redwood City, CA (US)

Correspondence Address: BUCHANAN, INGERSOLL & ROONEY PC, POST OFFICE BOX 1404, ALEXANDRIA, VA, 22313-1404, US

(73) Assignee: XONOPORT, INC., Santa Clara, CA (US)

(21) Appl. No.: 12/128,111

(22) Filed: May 28, 2008

Related U.S. Application Data
(63) Continuation of application No. 10/887,505, filed on Jul. 7, 2004.
Application No. 10/887,505 is a division of application No. 09/972,402, filed on Oct. 5, 2001, now abandoned.
(60) Provisional application No. 60/238,758, filed on Oct. 6, 2000.
(60) Provisional application No. 60/249,804, filed on Nov. 17, 2000.
(60) Provisional application No. 60/297,594, filed on Jun. 11, 2001.
(60) Provisional application No. 60/297,654, filed on Jun. 11, 2001.
(60) Provisional application No. 60/297,641, filed on Jun. 11, 2001.

(51) Int. Cl. A61K 31/56; A61K 31/58; A61K 31/585

(52) U.S. Cl. 514/174; 514/182; 514/175

(57) ABSTRACT

Disclosed are methods for providing sustained systemic blood concentrations of orally delivered drugs. Still further, disclosed are compounds and pharmaceutical compositions that are used in such methods.

### CROSS-REFERENCE TO RELATED APPLICATIONS

This application is a divisional of U.S. application Ser. No. 09/972,402, filed Oct. 5, 2001 and claims priority under 35 U.S.C. 119(e) to U.S. Provisional Application Ser. No. 60/238,758, which was filed on Oct. 6, 2000; U.S. Provisional Application Ser. No. 60/249,804, which was filed on Nov. 17, 2000; U.S. Provisional Application Ser. No. 60/297,594, which was filed on Jun. 11, 2001; U.S. Provisional Patent Application No. 60/297,654, which was filed on Jun. 11, 2001; and U.S. Provisional Application Ser. No. 60/297,641, which was filed on Jun. 11, 2001; the disclosure of each application being incorporated herein in its entirety.

### BACKGROUND OF THE INVENTION

1. Field of the Invention

This invention is directed to methods for providing sustained release of orally delivered drugs to animals. Still further, this invention is directed to compounds and pharmaceutical compositions that are used in such methods.

2. State of the Art

The serum half-life or bioclearance of many orally delivered drugs is a significant factor in determining the frequency of dosing of these drugs to an animal patient. Ideally, the drug should have a serum half-life or bioclearance which permits once a day or, at most, twice a day dosing. However, there are many commercially successful, orally delivered drugs that require a more frequent dosing regimen. Notwithstanding the success of such drugs, patient convenience and compliance with drug dosing would be improved if the frequency of dosing could be reduced.

Prior attempts to reduce drug dosing include the use of transdermal delivery devices that provide a constant infusion of drug to the patient. These devices, conventionally used in the form of a patch, pose other problems, however, such as the site of application in an inconspicuous area of the body that is amenable to transdermal delivery.

Notwithstanding the success of transdermal delivery, the preferred route of drug administration is still oral delivery and, accordingly, methods for providing sustained release of orally delivered drugs would be particularly beneficial.

### SUMMARY OF THE INVENTION

This invention is directed to the surprising discovery that the enterohepatic circulation of an animal can be utilized to provide sustained release of orally delivered drugs thereby providing prolonged therapeutic or prophylactic systemic blood concentrations of the drugs.

Specifically, an orally delivered drug is conjugated to a moiety through a cleavable linker to provide for a compound that is translocated across the intestinal wall of an animal and can participate in the enterohepatic circulation of the animal. Such conjugation allows these compounds, when orally delivered to an animal, to traverse the intestinal wall and to cycle within the enterohepatic circulation of that animal.

The cleavable linker is selected relative to the activity, specificity and localization of enzymatic activity within tissues that comprise the enterohepatic circulation such that a portion of the linker is cleaved and delivered to the systemic blood circulation of the animal during each cycle through the enterohepatic circulation.

Accordingly, in one of its method aspects, this invention is directed to a method for achieving sustained therapeutic or prophylactic blood concentrations of a drug or active metabolite thereof in the systemic circulation of an animal which method comprises orally administering to said animal a compound of formula (I):

D-Y-T (I)

wherein D is a drug having therapeutic or prophylactic activity when delivered to the systemic circulation of said animal; T is a moiety selected to permit the compound of formula (I) or an active metabolite thereof to be translocated across the intestinal wall of an animal and participate in the enterohepatic circulation in said animal; and Y is a cleavable linker covalently connecting D to T wherein Y is selected such that a portion of the linker is cleaved to release drug D or active metabolite thereof during each cycle through the enterohepatic circulation whereupon sustained release of drug D in said animal is achieved.

Preferably, the cleavable linker Y is selected to provide for sustained release of drug D in said animal for a period of at least about 10% longer (more preferably at least 50% longer and still more preferably at least 100% longer) than the oral delivery of drug D itself.

Accordingly, another method aspect of this invention is directed to a method for achieving sustained therapeutic or prophylactic blood concentrations of a drug or active metabolite thereof in the systemic circulation of an animal which method comprises orally administering to said animal a compound of formula (I):

D-Y-T (I)

wherein D is a drug having therapeutic or prophylactic activity when delivered to the systemic circulation of said animal; T is a moiety selected to permit the compound of formula (I) or a active metabolite thereof to be translocated across the intestinal wall of an animal and participate in the enterohepatic circulation in said animal; and Y is a cleavable linker covalently connecting D to T wherein Y is selected to provide for sustained release of drug D in said animal for a period of at least about 10% longer (more preferably at least 50% longer and still more preferably at least 100% longer) than the oral delivery of drug D itself.

As noted above, the selection of linker is preferably made relative to the activity, specificity and localization of enzymatic activity within tissues that comprise the enterohepatic circulation such that the drug is released at a site from where it is made available to the systemic circulation. For example, in one preferred embodiment, the linker is selected to contain one or more ester groups that permit cleavage of such groups by endogenous esterases within such tissues. In another preferred embodiment, the linker is selected to contain one or more amide groups which amide groups permit cleavage of such groups by endogenous proteases.

The methods of this invention are preferably achieved by use of compounds of formula (I) above. Accordingly, in one of its composition aspects, this invention is directed to a compound of formula (I):

D-Y-T (I)

wherein D is a drug having therapeutic or prophylactic activity when delivered to the systemic circulation of said animal; T is a moiety selected to permit the compound of formula (I) or a active metabolite thereof to be translocated across the intestinal wall of an animal and participate in the enterohepatic circulation in said animal; and Y is a cleavable linker covalently connecting D to T wherein Y is selected such that a portion of the linker is cleaved to release drug D or active metabolite thereof during each cycle through the enterohepatic circulation whereupon sustained release of drug D in said animal is achieved.

Preferably D is a drug containing at least one moiety selected from the group consisting of hydroxyl, thiol, NH, carboxylic acid (or salt thereof), phosphonic acid (or salt thereof) and phosphoric acid (or salt thereof).

The linker group Y is more preferably represented by the formula —X—[Y*]-Z- where X is the linker chemistry for attachment to the drug; Y* is a covalent bond or a linker moiety; and Z is the linker chemistry for attachment to T.

Preferably X is selected from the group consisting —OC(O)—, —OC(O)NR⁷—, —OC(O)OCR¹¹R¹²O—, —OC(O)OCR¹¹R¹²OC(O)—, —OC(O)OCR¹¹R¹²OC(O)O—, —OC(O)OCR¹¹R¹²OC(O)NR⁷—, —SC(O)—, —NR⁷C(O)O—, —NR⁷C(O)—, —NR⁷C(O)OCR¹¹R¹²OC(O)—, —NR⁷C(O)OCR¹¹R¹²OC(O)O—, —NR⁷CH₂NR⁷C(O)—, —C(O)O—, —C(O)S—, —C(O)NR⁷—, —C(O)NR⁷C(O)R⁸—, —C(O)OCR¹¹R¹²O, —C(O)OCR¹¹R¹²OC(O)—, —C(O)OCR¹¹R¹²OC(O)O—, —C(O)OCH₂C(O)NR⁷—, —C(O)OCH₂CH₂NR⁷C(O)—, —C(O)OCH₂NR⁷C(O)—, —C(O)OCR¹¹R¹²OC(O)NR⁷—, —P(O)(OR⁶)O—, —P(O)(OR⁶)NR⁷—, —P(O)(OR⁶)OCR¹¹R¹²O—, —P(O)(OR⁶)OCR¹¹R¹²OC(O)—, —P(O)(OR⁶)OCR¹¹R¹²OC(O)O—, —P(O)(OR⁶)OCR¹¹R¹²OC(O)NR⁷—, with the underlined atom being derived from the hydroxyl, thiol, NH, carboxylic acid (or salt thereof), phosphonic acid (or salt thereof) or phosphoric acid (or salt thereof) moiety of the drug;

wherein R⁶ is selected from the group consisting alkyl, substituted alkyl, aryl and substituted aryl; each R⁷ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Preferably Z is selected from the group consisting of a bond, —O—, —S—, —C(O)O—, —OC(O)O—, —NR⁷C(O)O—, —OC(O)NR⁷—, —OP(O)(OR⁶)O—, —P(O)(OR⁶)O—, —NR⁷P(O)(OR⁶)O—, —C(O)NR⁷—, —NR⁷C(O)NR⁷—, —NR⁷C(O)NR⁷—, —S(O)₂NR⁷—, —S(O)—, —S(O)₂—, —C(O)S—, —ON═, —C(O)ON═, —NR⁷C(O)ON═, —C(O)OCR¹¹R¹²ON═, and a C═C linkage, wherein R⁶-R¹² are defined as above.

Preferably Y* is a bond or a bivalent hydrocarbyl radical of 1 to 18 atoms having at least one alkylene, alkenylene or alkynylene group, with said at least one alkylene, alkenylene or alkynylene group optionally replaced with —O—, —S—, —NR⁷—, —C(O)—, —C(S)—, —OC(O)—, —C(O)O, —SC(O)—, —C(O)S—, —SC(S)—, —C(S)S—, —C(O)NR⁷—, —NR⁷C(O)—, arylene, substituted arylene, cycloalkylene, substituted cycloalkylene, cycloalkenylene, substituted cycloalkenylene, bivalent heterocyclic group or substituted bivalent heterocyclic group, wherein R⁷ is defined as above.

Y* is also preferably represented by the formula:

(R³)_{f}(R⁴)_{g}(R⁵)ₕ

where each of R³, R⁴ and R⁵ are independently selected from the group consisting of alkylene, substituted alkylene, alkenylene, substituted alkenylene, alkynylene, substituted alkynylene, cycloalkylene, substituted cycloalkylene, cycloalkenylene, substituted cycloalkenylene, arylene, substituted arylene, heteroarylene, substituted heteroarylene, heterocyclene and substituted heterocyclene; and each of f, g and h are independently an integer from 0 to 3. More preferably, Y* is alkylene, alkenylene or alkynylene.

The compounds described above are preferably administered as pharmaceutical compositions comprising the drug/cleavable linker/transporter compounds described above and a pharmaceutically acceptable excipient.

### BRIEF DESCRIPTION OF THE DRAWINGS

FIG. 1 illustrates the enterohepatic circulation in mammals with key transporter proteins mediating bile acid circulation.

FIG. 2 illustrates bile acid prodrug derivatives (I-a) and (I-b) for sustained release of drugs using cholic acid or cholic acid derivatives as the substrate suitable for enterohepatic circulation.

FIG. 3 illustrates preferred derivatives of bile acids modified at the C-3 position thereof to include a pharmaceutically active drug for sustained release.

FIG. 4 illustrates preferred derivatives of bile acids modified at the C-24 position thereof to include a pharmaceutically active drug for sustained release.

FIG. 5 illustrates GABA analog and L-DOPA derivatives suitable for conjugation to the C-3 and/or the C-24 position of bile acids for sustained release of the GABA analog or L-DOPA.

FIG. 6 illustrates catechol protection strategies applicable for L-DOPA conjugated to bile acids such that the resulting conjugate will participate within the enterohepatic circulation and provide sustained release of L-DOPA.

FIG. 7 illustrates prodrugs for enterohepatic circulation via the intestinal and liver anion transporters that will provide for sustained release of the conjugated drug in vivo.

FIG. 8 depicts a strategy for achieving enterohepatic recycling of a prodrug or other compound by exploiting intestinal absorption by the peptide transporter, PEPT1, coupled with hepatic uptake and biliary secretion by anion transporters from the OATP and ABC transporter families respectively (e.g. specifically OATP1 and/or OATP2 in the sinusoidal membrane and MRP2 in the canalicular membrane of the hepatocyte).

FIG. 9 illustrates glutathione mimetic prodrugs that undergo enterohepatic circulation via the mechanism shown in FIG. 8 and are slowly hydrolyzed in vivo to provide sustained release of drug D.

FIGS. 10-13 illustrate synthetic protocols for the synthesis of several prodrugs of gabapentin that will participate in the enterohepatic circulation and provide for sustained release of the conjugated drug in vivo.

FIG. 14 illustrates synthetic protocols for the synthesis of several prodrugs of L-DOPA that will participate in the enterohepatic circulation and provide for sustained release of the conjugated drug in vivo.

FIGS. 15-17 illustrate the synthesis of several glutathione mimetic conjugates capable of undergoing enterohepatic circulation via the intestinal peptide transporter and liver anion transporters.

### DETAILED DESCRIPTION OF THE INVENTION

This invention provides compositions and methods for providing sustained release of drugs when orally delivered to an animal. However, prior to describing this invention in further detail, the following terms will first be defined:

### DEFINITIONS

As used herein, the term “animal” refers to various species such as mammalian and avian species including, by way of example, humans, cattle, sheep, horses, dogs, cats, turkeys, chicken, and the like. Preferably, the animal is a mammal and even more preferably is a human.

The term “orally delivered drugs” refer to drugs which are administered to an animal in an oral form, preferably, in a pharmaceutically acceptable diluent. Oral delivery includes ingestion of the drug as well as oral gavage of the drug.

The term “systemic bioavailability” refers to the rate and extent of systemic exposure to a drug or an active metabolite thereof as reflected by the area under the systemic blood concentration versus time curve.

The term “translocation across the intestinal wall” refers to movement of a drug or drug conjugate by a passive or active mechanism, or both, across an epithelial cell membrane of any region of the gastrointestinal tract.

“Active metabolite of a drug” refers to products of in vivo modification of the compounds of this invention that have therapeutic or prophylactic effect

“Therapeutic or prophylactic blood concentrations” refers to systemic exposure to a sufficient concentration of a drug or an active metabolite thereof over a sufficient period of time to effect disease therapy or to prevent the onset or reduce the severity of a disease in the treated animal.

“Sustained release” refers to release of a therapeutic or prophylactic amount of the drug or an active metabolite thereof into the systemic blood circulation over a prolonged period of time relative to that achieved by oral administration of a conventional formulation of the drug.

“Tissue of the enterohepatic circulation” refers to the blood, plasma, intestinal contents, intestinal cells, liver cells, biliary tract or any fraction, suspension, homogenate, extract or preparation thereof.

“Conjugating” refers to the formation of a covalent bond.

“Bile acid transport system” refers to any membrane transporter protein capable of causing a bile acid or a derivative thereof to be translocated across a membrane of a cell of the gastrointestinal tract or liver.

“Active transport or active transport mechanism” refers to the movement of molecules across cellular membranes that:

a) is directly or indirectly dependent on an energy mediated process (i.e., driven by ATP hydrolysis, ion gradient, etc);

or

b) occurs by facilitated diffusion mediated by interaction with specific transporter proteins;

or

c) occurs through a modulated solute channel.

“A moiety selected to permit a compound of this invention or an active metabolite thereof to be translocated across the intestinal wall of an animal and participate in the enterohepatic circulation of said animal” refers to compounds which, when conjugated to the drug/cleavable linker moiety, are translocated across the intestinal wall through an active or passive transport mechanism and are subsequently substrates for participation in the enterohepatic circulation. Evaluation of which candidate compounds can be so translocated across the intestinal wall can be conducted by the in vitro assays set forth in Examples 33 and 34 below.

“Cleavable linker” refers to either a covalent bond between drug, D, and transporter, T, which bond is labile (and, hence, cleavable) and to discrete linkers that contain one or more functional groups that permit cleavage of such a bond or group in vivo by, for example, endogenous enzymes, such as esterases and amidases. Preferably, the cleavable linker is a functional group subject to cleavage at the point of attachment of the linker to the drug moiety, D, such that upon cleavage, free drug is released. The cleavable linker preferably comprises one or more functional groups such as ester groups, amide groups, glycolamide ester groups, amidomethyl esters, acyloxyalkyl esters, alkoxycarbonyloxyalkyl esters, and the like.

The term “derivatives of L-DOPA” preferably refers to L-DOPA molecules wherein:

a) a hydrogen atom of the amino group of the L-DOPA molecule is replaced with

—C(O)R²⁰⁴, —C(O)OR²⁰⁵ or an amino acid group, wherein R²⁰⁴ is selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, and R²⁰⁵ is selected from the group consisting of alkyl, substituted alkyl, cycloalkyl, substituted cycloalkyl, aryl, substituted aryl, aralkyl, substituted aralkyl, heteroaryl and substituted heteroaryl; and/or

b) one or two hydrogen atoms of the two —OH groups of the catechol group of the L-DOPA molecule are replaced with —C(O)R²⁰⁴, —C(O)OR²⁰⁵ and/or —OCR²⁰³R²⁰⁴OC(O)R²⁰⁵ wherein R²⁰³ and R²⁰⁴ independently are members selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, or R²⁰³ and R²⁰⁴ together with the carbon atom to which they are attached form a cycloalkyl, substituted cycloalkyl, heterocycle or substituted heterocyclic ring, or the two —OH groups of the catechol group of the L-DOPA molecule are protected with a 5-membered cyclic carbonate or 2,3-dioxo-1,4-dioxane ortho fused with a benzene ring of the catechol group of the L-DOPA molecule; and/or

c) the OH group of the carboxyl moiety is replaced by —OR²⁰⁴

with the proviso that one of the amino hydrogen atoms, the hydroxyl group of the carboxyl moiety or the hydrogen atom of one of the hydroxyl groups of the catechol is removed to form a covalent bond to either Y^{a} or Y^{b}.

“GABA analog” preferably refers to a moiety of the following formula:

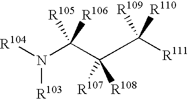

wherein

R¹⁰³ is selected from the group consisting of hydrogen, an amino-protecting group, or a covalent bond linking the moiety to either Y^{a} or Y^{b};

R¹⁰⁴ is hydrogen, or R¹⁰⁴ and R¹⁰⁹ together with the atoms to which they are attached form a heterocyclic ring;

R¹⁰⁵ and R¹⁰⁶ are independently selected from the group consisting of hydrogen, alkyl, substituted alkyl, alkenyl, substituted alkenyl, alkynyl, aryl, substituted aryl, heteroaryl and substituted heteroaryl;

R¹⁰⁷ and R¹⁰⁸ are independently selected from the group consisting of hydrogen, alkyl, substituted alkyl, alkenyl, alkynyl, aryl, substituted aryl, heteroaryl and substituted heteroaryl, or R¹⁰⁷ and R¹⁰⁸ together with the atoms to which they are attached form a cycloalkyl, substituted cycloalkyl, heterocyclic or substituted heterocyclic ring;

R¹⁰⁹ is selected from the group consisting of hydrogen, alkyl, substituted alkyl, alkenyl, alkynyl, aryl, substituted aryl, heteroaryl and substituted heteroaryl;

R¹¹⁰ is selected from the group consisting of hydrogen, alkyl, substituted alkyl, alkenyl, alkynyl, aryl, substituted aryl, heteroaryl and substituted heteroaryl;

R¹¹¹ is selected from the group consisting of carboxylic acid, carboxylic amide, carboxylic ester, sulfonamide, phosphonic acid, acidic heterocycle, sulfonic acid, hydroxamic acid and C(O)R¹¹²;

R¹¹² is a covalent bond linking the GABA analog moiety to either Y^{a} or Y^{b}, provided only one of R¹⁰³ and R¹¹² links the moiety to Y^{a} or Y^{b}.

“Acidic heterocycle” refers to a reprotonatable heterocycle having a pKa less than 7.0. Examples of such heterocycles include the following:

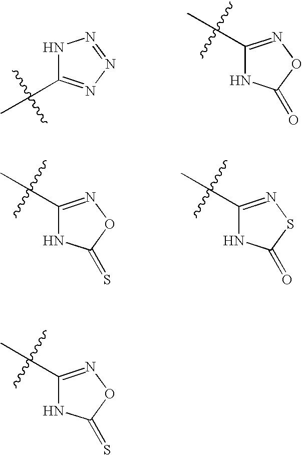

“An inhibitor of L-aromatic amino acid decarboxylase” preferably refers to L-aromatic amino acid decarboxylase inhibitors such as carbidopa and benzserazide optionally with a hydrogen atom of the amino or the hydrazido group of the L-aromatic amino acid decarboxylase inhibitor replaced with —C(O)R³⁰⁴, —C(O)OR³⁰⁵ or an amino acid group, wherein R³⁰⁴ is selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, and R³⁰⁵ is selected from the group consisting of alkyl, substituted alkyl, cycloalkyl, substituted cycloalkyl, aryl, substituted aryl, aralkyl, substituted aralkyl, heteroaryl and substituted heteroaryl; and/or optionally with one or two hydrogen atoms of the two —OH groups of the catechol or the three —OH groups of the pyrogallol group of the L-aromatic amino acid decarboxylase inhibitor are replaced with —C(O)R³⁰⁴, C(O)OR³⁰⁵ and/or —OCR³⁰³R³⁰⁴OC(O)R³⁰⁵ wherein R³⁰³ and R³⁰⁴ independently are members selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, or R³⁰³ and R³⁰⁴ together with the carbon atom to which they are attached form a cycloalkyl, substituted cycloalkyl, heterocycle or substituted heterocyclic ring; or optionally with two adjacent —OH groups of the catechol or pyrogallol group protected with a 5-membered cyclic carbonate or 2,3-dioxo-1,4-dioxane ortho fused with a benzene ring of the catechol or pyrogallol group; and/or

the OH group of the carboxyl moiety is replaced by —OR³⁰⁴ with the proviso that one of the amino hydrogen atoms, the hydroxyl group of the carboxyl moiety or the hydrogen atom of one of the hydroxyl groups of the catechol/pyrogallol is removed to form a covalent bond to either Y^{a} or Y^{b}.

“Catechol O-methyl transferase inhibitor” preferably refers to catechol O-methyl transferase inhibitors such as entacapone, nitecapone and tolcapone optionally with one or two hydrogen atoms of two hydroxyl groups of the catechol group replaced with —C(O)R³⁰⁴, —C(O)OR³⁰⁵ and/or —OCR³⁰³R³⁰⁴OC(O)R³⁰⁵ wherein R³⁰³ and R³⁰⁴ independently are members selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, or R³⁰³ and R³⁰⁴ together with the carbon atom to which they are attached form a cycloalkyl, substituted cycloalkyl, heterocycle or substituted heterocyclic ring, R³⁰⁵ is selected from the group consisting of alkyl, substituted alkyl, cycloalkyl, substituted cycloalkyl, aryl, substituted aryl, aralkyl, substituted aralkyl, heteroaryl and substituted heteroaryl, or the OH group of the carboxyl moiety is replaced by —OR³⁰⁴, with the proviso that one of the amino hydrogen atoms or the hydrogen atom of one of the hydroxyl groups of the catechol is removed to form a covalent bond to either Y^{a} or Y^{b}.

“Linear oligopeptide” refers to an amide oligomer comprising either a terminal amino group or a terminal carboxylic acid group or (preferably) both a terminal amino group and a terminal carboxylic acid group, which oligomer is formed by condensation of the terminal amino residue of at least one amino acid (or GABA analog) with the terminal carboxylic acid residue of at least a second amino acid (or GABA analog). In addition to the GABA analog, the amino acids comprising the oligopeptide are optionally either α-amino acids, β-amino acids, or a mixture of α-amino acids and β-amino acids. Note that when an α-amino acid additionally contains either a β-amino group or a β-carboxylic acid group (e.g. as in aspartic acid) a linear oligopeptide formed from such an amino acid is intended to imply that it is the α-amine or α-carboxylic acid moiety (or both) of such residue that is involved in amide formation.

“α-Amino acids” are molecules of the formula: HNR⁵⁰—CR⁵¹R⁵²—C(O)OH

wherein:
R⁵⁰ is hydrogen or R⁵⁰ and R⁵¹ together with the atoms to which they are attached form a heterocyclyl ring;
R⁵¹ is hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R⁵¹ and R⁵² together with the atoms to which they are attached form a cycloalkyl, substituted cycloalkyl, heterocyclyl or substituted heterocyclyl ring.

“β-Amino acids” are molecules of formula:

HNR⁵⁰—(CR⁵¹R⁵²)—(CR⁵³R⁵⁴)—C(O)OH:

wherein:
R⁵⁰ is hydrogen or R⁵⁰ and R⁵¹ together with the atoms to which they are attached form a heterocyclyl ring;
R⁵¹ is hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R⁵¹ and R⁵² together with the atoms to which they are attached form a cycloalkyl, substituted cycloalkyl, heterocyclyl or substituted heterocyclyl ring, or R⁵¹ and R⁵³ together with the atoms to which they are attached form a cycloalkyl, substituted cycloalkyl, heterocyclyl or substituted heterocyclyl ring;
R⁵² is hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl or substituted heteroaryl;
R⁵³ is hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R⁵³ and R⁵⁴ together with the atoms to which they are attached form a cycloalkyl, substituted cycloalkyl, heterocyclyl or substituted heterocyclyl ring;
R⁵⁴ is hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl or substituted heteroaryl.

“Naturally occurring amino acid” refers to any of the alpha-amino acids that are the chief components of proteins. The amino acids are either synthesized by living cells or are obtained as essential components of the diet. Such amino acids include, for example, the following: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“Derived from a compound” refers to a moiety that is structurally related to such a compound. The structure of the moiety is identical to the compound except at 1 or 2 positions. At these positions either a hydrogen atom attached to a heteroatom, or a hydroxyl moiety of a carboxylic, phosphonic, phosphoric or sulfonic acid group has been replaced with a covalent bond that serves as a point of attachment to another moiety. For example, the moiety:

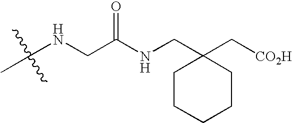

is derived from a linear oligopeptide comprising glycine and the drug gabapentin. In this moiety, a hydrogen atom has been replaced with a covalent bond. “Derived from a linear oligopeptide” is meant to specifically denote that the point of attachment is either the terminal amino group or the terminal acid group of the oligopeptide.

“Treating” a particular disease or disorder means reducing the number of symptoms or severity of symptoms of the disease, and/or reducing or limiting the further progression of the disease or disorder.

“Preventing” a disease or disorder means preventing or inhibiting the onset or occurrence of the disease or disorder.

The term “steroid” or “sterol” refers to the following core structure with the appropriate numbering system inserted therein:

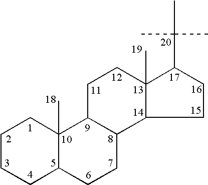

Accordingly, cholic acid which has the structure:

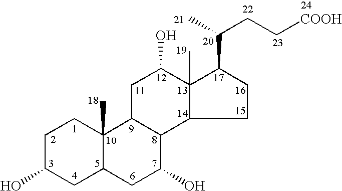

is numbered as shown above.

“Practical dosage regimen” refers to a schedule of drug administration that is practical for a patient to comply with. For human patients, a practical dosage regimen for an orally administered drug is likely to be an aggregate dose of less than 10 g/day.

“Alkyl” refers to alkyl groups preferably having from 1 to 20 carbon atoms and more preferably 1 to 6 carbon atoms. This term is exemplified by groups such as methyl, t-butyl, n-heptyl, octyl, dodecyl and the like.

“Substituted alkyl” refers to an alkyl group, preferably of from 1 to 20 carbon atoms, having from 1 to 5 substituents selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted aryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₄-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)-cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— wherein alkyl, substituted alkyl, alkenyl, substituted alkyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acylamino” refers to the group —C(O)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiocarbonylamino” refers to the group —C(S)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form, together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” refers to alkenyl group preferably having from 2 to 20 carbon atoms and more preferably 2 to 6 carbon atoms and having at least 1 and preferably from 1-2 sites of alkenyl unsaturation.

“Substituted alkenyl” refers to alkenyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynyl” refers to alkynyl group preferably having from 2 to 20 carbon atoms and more preferably 3 to 6 carbon atoms and having at least 1 and preferably from 1-2 sites of alkynyl unsaturation.

“Substituted alkynyl” refers to alkynyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkylene” refers to a divalent alkylene group preferably having from 1 to 20 carbon atoms and more preferably 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” refers to alkylene groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkenylene” refers to a divalent alkenylene group preferably having from 2 to 20 carbon atoms and more preferably 1 to 6 carbon atoms and having from 1 to 2 sites of alkenyl unsaturation. This term is exemplified by groups such as ethenylene (—CH═CH—), propenylene (—CH₂CH═CH—), and the like.

“Substituted alkenylene” refers to alkenylene groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynylene” refers to a divalent alkynylene group preferably having from 2 to 20 carbon atoms and more preferably 1 to 6 carbon atoms and having from 1 to 2 sites of alkynyl unsaturation. This term is exemplified by groups such as ethynylene, propynylene and the like.

“Substituted alkynylene” refers to alkynylene groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Amidino” refers to the group H₂NC(═NH)— and the term “alkylamidino” refers to compounds having 1 to 3 alkyl groups (e.g., alkylHNC(═NH)—).

“Thioamidino” refers to the group RSC(═NH)— where R is hydrogen or alkyl.

“Aminoacyl” refers to the groups —NRC(O)alkyl, —NRC(O)substituted alkyl, —NRC(O)cycloalkyl, —NRC(O)substituted cycloalkyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the groups —NRC(O)O-alkyl, —NRC(O)O-substituted alkyl, —NRC(O)O-alkenyl, —NRC(O)O-substituted alkenyl, —NRC(O)O-alkynyl, —NRC(O)O-substituted alkynyl, —NRC(O)O-cycloalkyl, —NRC(O)O-substituted cycloalkyl, —NRC(O)O-aryl, —NRC(O)O-substituted aryl, —NRC(O)O-heteroaryl, —NRC(O)O-substituted heteroaryl, —NRC(O)O-heterocyclic, and —NRC(O)O-substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxycarbonylamino” refers to the groups —OC(O)NH₂, —OC(O)NRR, —OC(O)NR-alkyl, —OC(O)NR-substituted alkyl, —OC(O)NR-alkenyl, —OC(O)NR-substituted alkenyl, —OC(O)NR-alkynyl, —OC(O)NR-substituted alkynyl, —OC(O)NR-cycloalkyl, —OC(O)NR-substituted cycloalkyl, —OC(O)NR-aryl, —OC(O)NR-substituted aryl, —OC(O)NR-heteroaryl, —OC(O)NR-substituted heteroaryl, —OC(O)NR-heterocyclic, and —OC(O)NR-substituted heterocyclic where R is hydrogen, alkyl or where each R is joined to form, together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxythiocarbonylamino” refers to the groups —OC(S)NH₂, —OC(S)NRR, —OC(S)NR-alkyl, —OC(S)NR-substituted alkyl, —OC(S)NR-alkenyl, —OC(S)NR-substituted alkenyl, —OC(S)NR-alkynyl, —OC(S)NR-substituted alkynyl, OC(S)NR-cycloalkyl, —OC(S)NR-substituted cycloalkyl, —OC(S)NR-aryl, —OC(S)NR-substituted aryl, —OC(S)NR-heteroaryl, —OC(S)NR-substituted heteroaryl, —OC(S)NR-heterocyclic, and —OC(S)NR-substituted heterocyclic where R is hydrogen, alkyl or where each R is joined to form together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the groups —NRC(O)NRR, —NRC(O)NR-alkyl, —NRC(O)NR-substituted alkyl, —NRC(O)NR-alkenyl, —NRC(O)NR-substituted alkenyl, —NRC(O)NR-alkynyl, —NRC(O)NR-substituted alkynyl, —NRC(O)NR-aryl, —NRC(O)NR-substituted aryl, —NRC(O)NR-cycloalkyl, —NRC(O)NR-substituted cycloalkyl, —NRC(O)NR-heteroaryl, and —NRC(O)NR-substituted heteroaryl, —NRC(O)NR-heterocyclic, and —NRC(O)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted allyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the groups —NRC(S)NRR, —NRC(S)NR-alkyl, —NRC(S)NR-substituted alkyl, —NRC(S)NR-alkenyl, —NRC(S)NR-substituted alkenyl, —NRC(S)NR-alkynyl, —NRC(S)NR-substituted alkynyl, —NRC(S)NR-aryl, —NRC(S)NR-substituted aryl, —NRC(S)NR-cycloalkyl, —NRC(S)NR-substituted cycloalkyl, —NRC(S)NR-heteroaryl, and —NRC(S)NR-substituted heteroaryl, —NRC(S)NR-heterocyclic, and —NRC(S)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to a monovalent unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like). Preferred aryls include phenyl and naphthyl.

“Substituted aryl” refers to aryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OS(O)₂—NRR where R is hydrogen or alkyl, —N S(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Arylene” refers to a divalent unsaturated aromatic carbocyclic group of from 6 to 14 carbon atoms having a single ring (e.g., phenylene) or multiple condensed rings (e.g., naphthylene or anthrylene) which condensed rings may or may not be aromatic. Preferred arylenes include phenylene and naphthylene.

Substituted arylene refers to arylene groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OS(O)₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted allyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with

—SO₂NRR where R is hydrogen or alkyl.

“Aryloxy” refers to the group aryl-O— which includes, by way of example, phenoxy, naphthoxy, and the like.

“Substituted aryloxy” refers to substituted aryl-O— groups.

“Aryloxyaryl” refers to the group-aryl-O-aryl.

“Substituted aryloxyaryl” refers to aryloxyaryl groups substituted with from 1 to 3 substituents on either or both aryl rings selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Cycloalkyl” refers to cyclic alkyl groups of from 3 to 8 carbon atoms having a single cyclic ring including, by way of example, cyclopropyl, cyclobutyl, cyclopentyl, cyclooctyl and the like. Excluded from this definition are multi-ring alkyl groups such as adamantanyl, etc.

“Cycloalkenyl” refers to cyclic alkenyl groups of from 3 to 8 carbon atoms having a single cyclic ring.

“Substituted cycloalkyl” and “substituted cycloalkenyl” refers to an cycloalkyl or cycloalkenyl group, preferably of from 3 to 8 carbon atoms, having from 1 to 5 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Cycloalkylene” refers to divalent cyclic alkylene groups of from 3 to 8 carbon atoms having a single cyclic ring including, by way of example, cyclopropylene, cyclobutylene, cyclopentylene, cyclooctylene and the like.

“Cycloalkenylene” refers to a divalent cyclic alkenylene groups of from 3 to 8 carbon atoms having a single cyclic ring.

“Substituted cycloalkylene” and “substituted cycloalkenylene” refers to a cycloalkylene or cycloalkenylene group, preferably of from 3 to 8 carbon atoms, having from 1 to 5 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NR S(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Cycloalkoxy” refers to —O-cycloalkyl groups.

“Substituted cycloalkoxy” refers to —O-substituted cycloalkyl groups.

“Guanidino” refers to the groups —NRC(═NR)NRR, —NRC(═NR)NR-alkyl, —NRC(═NR)NR-substituted alkyl, —NRC(═NR)NR-alkenyl, —NRC(═NR)NR-substituted alkenyl, —NRC(═NR)NR-alkynyl, —NRC(═NR)NR-substituted alkynyl, —NRC(═NR)NR-aryl, —NRC(═NR)NR-substituted aryl, —NRC(═NR)NR-cycloalkyl, —NRC(═NR)NR-heteroaryl, —NRC(═NR)NR-substituted heteroaryl, —NRC(═NR)NR-heterocyclic, and —NRC(═NR)NR-substituted heterocyclic where each R is independently hydrogen and alkyl as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“N,N-Dimethylcarbamyloxy” refers to the group —OC(O)N(CH₃)₂.

“Guanidinosulfone” refers to the groups —NRC(═NR)NRSO₂-alkyl, —NRC(═NR)NRSO₂-substituted alkyl, —NRC(═NR)NRSO₂-alkenyl, —NRC(═NR)NRSO₂-substituted alkenyl, —NRC(═NR)NRSO₂-alkynyl, —NRC(═NR)NRSO₂-substituted alkynyl, —NRC(═NR)NRSO₂-aryl, —NRC(═NR)NRSO₂-substituted aryl, —NRC(═NR)NRSO₂-cycloalkyl, —NRC(═NR)NRSO₂-substituted cycloalkyl, —NRC(═NR)NRSO₂-heteroaryl, and —NRC(═NR)NRSO₂-substituted heteroaryl, —NRC(═NR)NRSO₂-heterocyclic, and —NRC(═NR)NRSO₂-substituted heterocyclic where each R is independently hydrogen and alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Halo” or “halogen” refers to fluoro, chloro, bromo and iodo and preferably is either chloro or bromo.

“Heteroaryl” refers to an aromatic carbocyclic group of from 2 to 10 carbon atoms and 1 to 4 heteroatoms selected from the group consisting of oxygen, nitrogen and sulfur within the ring. Such heteroaryl groups can have a single ring (e.g., pyridyl or furyl) or multiple condensed rings (e.g., indolizinyl or benzothienyl). Preferred heteroaryls include pyridyl, pyrrolyl, indolyl and furyl.

“Substituted heteroaryl” refers to heteroaryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, OS(O)₂-alkyl, —S(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with

—SO₂NRR where R is hydrogen or alkyl.

“Heteroarylene” refers to a divalent aromatic carbocyclic group of from 2 to 10 carbon atoms and 1 to 4 heteroatoms selected from the group consisting of oxygen, nitrogen and sulfur within the ring. Such heteroarylene groups can have a single ring (e.g., pyridylene or furylene) or multiple condensed rings (e.g., indolizinylene or benzothienylene). Preferred heteroarylenes include pyridylene, pyrrolylene, indolylene and furylene.

“Substituted heteroarylene” refers to heteroarylene groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂— substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-hetero cyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl.

“Substituted heterocyclic” refers to heterocycle groups which are substituted with from 1 to 3 substituents selected from the group consisting of oxo (═O), thioxo (═S), alkoxy, substituted alkoxy, acyl, acyl, amino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, —C(O)O-aryl, —C(O)O-substituted aryl, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and

—SO₂NRR where R is hydrogen or alkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclene” refers to a divalent saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl.

“Substituted heterocyclene” refers to heterocyclene groups which are substituted with from 1 to 3 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, —C(O)O-aryl, —C(O)O-substituted aryl, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or allyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Heterocyclyloxy” refers to the group —O-heterocyclic and “substituted heterocyclyloxy” refers to the group —O-substituted heterocyclic.

“Thiol” refers to the group —SH.

“Thioalkyl” refers to the groups —S-alkyl

“Substituted thioalkyl” refers to the group —S-substituted alkyl.

“Thiocycloalkyl” refers to the groups —S-cycloalkyl.

“Substituted thiocycloalkyl” refers to the group —S-substituted cycloalkyl.

“Thioaryl” refers to the group —S-aryl and “substituted thioaryl” refers to the group —S-substituted aryl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

“Pharmaceutically acceptable salt” refers to pharmaceutically acceptable salts of a compound of this invention which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

### Compound Preparation

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Second Edition, Wiley, New York, 1991, and references cited therein.

Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The compounds of this invention can be prepared by either by direct conjugation of a drug, D, to a transporter, T, wherein the resulting covalent bond is cleavable in vivo or by covalently coupling a difunctionalized linker precursor with a drug and a suitable transporter compound. The linker precursor is selected to contain at least one reactive functionality that is complementary to at least one reactive functionality on the drug and at least one reactive functionality on the transporter compound. Such complementary reactive groups are well known in the art as illustrated below:

COMPLEMENTARY BINDING CHEMISTRIES

| First Reactive Group | Second Reactive Group | Linkage |
|---|---|---|
| hydroxyl | carboxylic acid | ester |
| amine | carboxylic acid | amide |
| hydroxyl | isocyanate | urethane |
| amine | epoxide | hydroxyamine |
| sulfonyl halide | amine | sulfonamide |
| hydroxyl | alkyl/aryl halide | ether |
| aldehyde | amine/NaCNBH₄ | amine |
| ketone | amine/NaCNBH₄ | amine |
| amine | isocyanate | urea |

Suitable linker precursors include, by way of example, dicarboxylic acids, disulfonylhalides, dialdehydes, diketones, dihalides, diisocyanates, diamines, diols, mixtures of carboxylic acids, sulfonylhalides, aldehydes, ketones, halides, isocyanates, amines and diols. In each case, the linker precursor is reacted with a complementary functionality on the drug and on the transporter compound to form a compound of this invention.

Examples of dicarboxylic acids useful as cleavable linkers herein include, for example, succinic acid, maleic acid, etc.

Examples of diols include, for example, polyoxyalkylene compounds of the general formula HO(alkylene-O)ₐ—H where alkylene is as defined herein and a is an integer from 1 to 20.

Examples of diamines include, for example, polyalkylene amine compounds of the general formula H₂N(alkylene-NH)ₐ—H where alkylene is as defined herein and a is an integer from 1 to 20. Reaction of the complementary functional groups to form a covalent linkage follows conventional chemical reactions. For example, drugs with a carboxylic acid group or an amine group (as described above) can be reacted under conventional conditions with an amine or a carboxylic acid to form an amide bond using conventional coupling techniques and reagents, such carbodiimides, BOP reagent and the like which are well known in the peptide art. Alternatively, amine and hydroxyl groups can be reacted with an isocyanate under conventional conditions to form a urea or carbamate linkage respectively.

The examples set forth below illustrate protocols for the synthesis of specific drugs/cleavable linker/transporter compounds.

The transporter moiety, T, is selected to permit the drug/cleavable linker/transporter compound or an active metabolite thereof to:

translocate across the intestinal wall of an animal; and

participate in the enterohepatic circulation.

Passage of such a compound through the enterohepatic circulation in this manner requires the compound to minimally traverse 4 key membrane barriers, comprising (i) the apical membrane of enterocytes (i.e. uptake from the intestinal lumen); (ii) the basolateral membrane of enterocytes (secretion into the blood); (iii) the sinusoidal (i.e. basolateral) membrane of hepatocytes (uptake into the liver from the portal blood); and (iv) the canalicular (i.e. apical) membrane of hepatocytes (i.e. secretion into the bile). The enterohepatic cycle is completed by bile flow back to the intestine wherein the compound is made available again for intestinal re-uptake. In many mammalian species (including man and other primates) bile flow is discontinuous, with the biliary contents being retained in a storage compartment (gall bladder) until compartment emptying (typically associated with food ingestion). In other species (e.g. rats), biliary secretions from the liver cycle continuously back to the intestine. Compound transfer across each of these membrane barriers may occur either by active transport (including facilitated diffusion) through interaction with membrane transporter protein(s) or via passive diffusion. Certain compounds may also be able to traverse one or more of these barriers via paracellular diffusional pathways.

Active transport mechanisms are particularly important contributors to compound flux within the enterohepatic cycle. Superfamilies and families of transporters, and individual transporters capable of contributing to the flux of prodrug, drug or drug metabolite, or other pharmacologically active molecules through one or more compartments of the enterohepatic circulation are listed below. The following examples are offered as illustrative, not restrictive. (Accession numbers in parentheses following each transporter)

Members of the family of sodium/bile acid cotransporters (family SLC10), including the Na-taurocholate cotransporting protein (NCTP or LBAT) (NM_{—}003049), and the apical bile acid transporter (IBAT or ASBT) (NM_{—}000452), and h-P3 (XM 013054).

Members of the family of organic cation/anion transporters (SLC22), including the organic cation transporters OCT1 (NM_{—}003057), OCT2 (NM_{—}003058), OCT3 (AF078749), OCTN1 (NM_{—}003059), OCTN2 (NM_{—}003060), ORCTL2 (AF037064), ORCTL3 (NM_{—}004256), ORCTL4 (NM_{—}004803), BOCT (NM-020372); and the organic anion transporters OAT-1 (NM_{—}004790), OAT-2 (NM_{—}006672), OAT-3 (NM_{—}004254), OAT-4 (AB026116), OAT-7 (NM-006672), OAT-8 (NM-019844).

Members of the organic ion/prostaglandin transporter family (SLC21), including OATP-A (OATP) (NM_{—}005075), OATP-B (NM_{—}007256), OATP-C (LST-1) (NM_{—}006446), OATP-D (NM_{—}013272), PGT (NM_{—}005630), OATP-F (NM_{—}017435), OATP-G (AX074150), OATP-H (AF205075).

Members of the proton/monocarboxylate cotransporter family (SLC16), including MCT1 (AAH01013), MCT2(XP_{—}013099), MCT3 (XP_{—}005733), MCT4 (XP_{—}002144), MCT5 (NP_{—}004686), MCT6 (XP_{—}017131), MCT7 (XP_{—}012127), MCT8 (XP_{—}009979), MCT9, MCT10.

Members of the proton/oligopeptide cotransporter family (SLC15), including PEPT1 (XM_{—}007063), PEPT2 (XM_{—}002922), PEPT3 (AV662097); and members of the peptide/histidine transporter family, including the peptide/histidine cotransporters PHT1 (W53019) and PHT2 (AB020598).

Members of the sodium/nucleoside cotransporter family (SLC28), including the concentrative nucleoside transporters CNT-1 (NM_{—}004213), CNT-2 (NM_{—}004212), CNT-3 (XP_{—}011759). Also the facilitated nucleoside transporter family, including the equilibrative nucleoside transporters ENT-1 (HSU81375), and ENT-2 (AF029385), ENT-3 (AAK00958).

Members of the D2/NBAT and 42F family (SLC3), including the amino acid transporters LAT1 (AF104032), LAT2 (AF135828), LAT3 (AF135829), LAT4 (NM_{—}004173), Y+LAT1 (D87432), Y+LAT2 (NM_{—}003982), HBAT (AF141289), HrBAT (L11696), NAAT-B (U53347), h4F2C (AB018010), ATBO+(AF151978).

Members of the sodium dicarboxylate/sulfate cotransporter family (SLC13) including NADC1 (NM_{—}003984), NADC2 (NM_{—}022444), NADC3 (AF154121).

Members of the families of vitamin and cofactor families such as the folate transporter family (SLC 19) which includes the reduced folate transporters (RFC) (P41440); and the sodium/ascorbate transporter family (JC7095); and the sodium/glucose cotransporter family (SLC5), including the sodium-dependent glucose transporter (SGLT-1) as well as the sodium-dependent multivitamin transporter (SMVT) (XP_{—}002430); and members of the facilitated glucose transporter family (SLC2).

Members of the ATP binding cassette transporter family (ABC transporters), including members of the ABC1 subfamily (A), including the cholesterol transporter ABCA1 (XM005567), and ABCA2 (AF178941), ABCA3 (XM007924), ABCA4 (XM001290), ABCA5 (AC005495), ABCA6 (AC005495), ABCA7 (XM00942612), ABCA8 (NM007168), ABCA9 (AC005922), ABCA10 (AC005495), ABCA11, ABCA12, ABCA13, ABCA14; the multidrug resistance (MDR)/TAP subfamily (B), including ABCB1 (MDR1, PgP)(XM004598), ABCB2 (XM004227), ABCB3 (XM004224), ABCB4 (MDR2/3)(NM000443), ABCB5 (AC002486), ABCB6 (XM002594), ABCB7 (NM004299), ABCB8 (XM004683), ABCB9 (NM019625), ABCB10 (XM001871), and ABCB11 (Bile salt export pump (BSEP or SGPG) (XM002644); the CFTR/multidrug resistance-associated (MRP) subfamily (C), including ABCC1 (MRP1) (NM004996), ABCC2 (MRP2 or cMOAT) (NM000392), ABCC3 (MRP3) (NM003786), ABCC4 (MRP4) (NM005845), ABCC5 (MRP5) (NM005688), ABCC6 (MRP6) (NM001171), ABCC7 (CFTR) (NM000492), and ABCC8 (NM000352), ABCC9 (NM005691), ABCC10 (AK000002), ABCC11, ABCC12, ABCC13; The ALD subfamily (D); And Subfamilies E (OABP), F (GCN20) and G (White).

Members of the long chain fatty acid transporter family (SLC27), the amino acid permease transporter family (SLC7), Urea transporter family (SLC14), including members 1 and 2 (NP_{—}056949; XP_{—}008765), and the polyamine transporters; and microsomal epoxide hydrolase (mEH) (AAF87738).

Vesicular/transcytosis transport systems including the receptor-mediated vitamin B12 transporter and the receptor-mediated folate transporter.

Intracellular binding proteins include
Intestinal bile acid binding protein (1-BABP)
Hepatocyte bile acid binding protein (HBAB)

Compounds which are actively transported by one or more of the above transporters are well known in the art and, if necessary, conjugation of two compounds can be made to effect participation in the enterohepatic circulation.

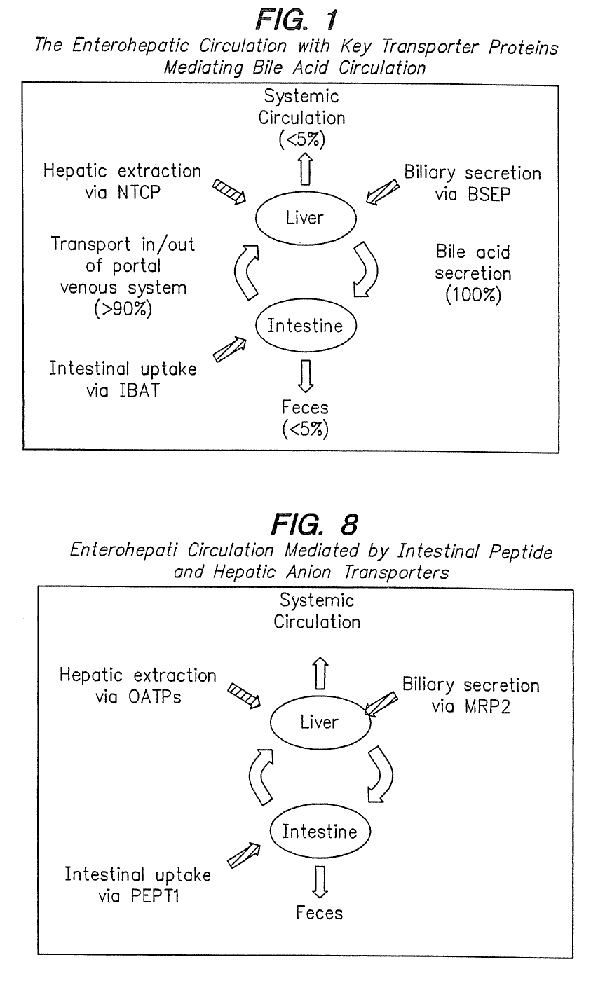

One group of known compounds which are substrates for IBAT and LBAT are the bile acids. Through the application of molecular biological tools, the key transporter proteins responsible for movement of the bile acid pool through the enterohepatic circulation have been defined in several species, as depicted in FIG. 1 (Kullak-Ublick et al, 2000). In man, the predominant circulating species are C-24 glycine and taurine conjugates of cholic acid. Transport of these conjugates via IBAT in the apical membrane of enterocytes, NTCP (or LBAT) in the sinusoidal membrane of hepatocytes and biliary secretion across the canalicular membrane of hepatocytes via the bile salt export pump BSEP and/or MRP2 are critical steps in the enterohepatic cycle. Canalicular transport is typically rate-limiting for the formation of bile, and the ˜160 kDa BSEP protein is an ATP-dependent export pump homologous with the MDR P-glycoproteins. The sodium-dependent cotransporters IBAT and NTCP share 36% sequence homology and are known to have distinct, but overlapping, substrate specificities. Other constituents of the bile acid pool are substrates for these transporters, including glycine and taurine conjugates of the “primary” bile acid chenodeoxycholic acid, as well as conjugates of the “secondary” bile acids deoxycholic acid and lithocholic acid, which are formed from the primary bile salts through bacterial metabolism within the intestine.

Structure-activity studies with a panel of naturally occurring and synthetic steroid derivatives have been used to elucidate pharmacophoric features of these molecules that are important for recognition by the ileal and hepatic transporters. One key observation is that the 3α-OH group present in all natural bile acids is not essential for high affinity interaction with the IBAT and NTCP transporters, making derivatization at C-3 of the steroid nucleus attractive for the design of bile acid-drug conjugates for enhancing intestinal drug absorption.

It is also recognized in the art that bile acids can be modified at other locations while still retaining their ability to participate in the enterohepatic circulation. For example, for optimal recognition by the Na⁺-dependent bile acid uptake systems in the hepatocyte and the ileocyte, the bile acids should contain a steroid moiety preferably with a cis-orientation of rings A and B, a negative charge in the side chain at position 17 and at least one hydroxyl group at position 3, 7 or 12 of the steroid nucleus. Thus, drug attachment to these bile acids can utilize any point of substitution provided that the resulting compound can translocate the intestinal wall.

One class of preferred bile acid conjugates is represented by the formula (I-a):

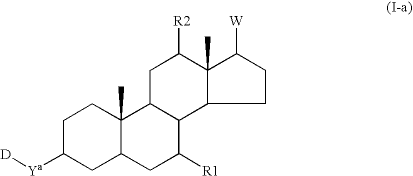

where Y^{a} is a cleavable linker;

D is a moiety derived from a drug;

R¹ is selected from the group consisting of hydrogen and OH;

R² is selected from the group consisting of hydrogen and OH; and

W is selected from the group consisting of —CH(CH₃)W′ where W′ is a substituted alkyl group containing a moiety which is negatively charged at physiological pH which moiety is selected from the group consisting of —COOH, —SO₃H, —SO₂H, P(O)(OR⁶)(OH), —OP(O)(OR⁶)(OH), —OSO₃H and pharmaceutically acceptable salts thereof;

R⁶ is selected from the group consisting of alkyl, substituted alkyl, aryl and substituted aryl;

wherein the compound of formula (I-a) above is a substrate for an intestinal bile acid transporter;

or pharmaceutically acceptable salts thereof.

Preferably D is a drug containing at least one moiety selected from the group consisting of hydroxyl, thiol, NH, carboxylic acid (or salt thereof), phosphonic acid (or salt thereof) and phosphoric acid (or salt thereof). The linker group —Y^{a}— is more preferably represented by the formula —X′—Y′-Z′- where X′ is the linker chemistry for attachment to the drug; Y′ is a covalent bond or a linker moiety; and Z′ is the linker chemistry for attachment to the bile acid.

Preferably X′ is selected from the group consisting —OC(O)—, —OC(O)NR⁷, —OC(O)OCR¹¹R¹²O—, —OC(O)OCR¹¹R¹²OC(O)—, —OC(O)OCR¹¹R¹²OC(O)O—, —OC(O)OCR¹¹R¹²OC(O)NR⁷—, —SC(O)—, —NR⁷C(O)O—, —NR⁷C(O)—, —NR⁷C(O)OCR¹¹R¹²OC(O)—, —NR⁷C(O)OCR¹¹R¹²OC(O)O—, —NR⁷CH₂NR⁷C(O)—, —C(O)O—, —C(O)S—, —C(O)NR⁷—, —C(O)NR⁷C(O)R⁸—, —C(O)OCR¹¹R¹²O—, —C(O)OCR¹¹R¹²OC(O)—, —C(O)OCR¹¹R¹²OC(O)O—, —C(O)OCH₂C(O)NR⁷—, —C(O)OCH₂CH₂NR⁷C(O)—, —C(O)OCH₂NR⁷C(O)—, —C(O)OCR¹¹R¹²OC(O)NR⁷—, —P(O)(OR⁶)O—, —P(O)(OR⁶)NR⁷—, —P(O)(OR⁶)OCR¹¹R¹²O—, —P(O)(OR⁶)OCR¹¹R¹²OC(O)—, —P(O)(OR⁶)OCR¹¹R¹²OC(O)O—, —P(O)(OR⁶)OCR¹¹R¹²OC(O)NR⁷—, with the underlined atom being derived from the hydroxyl, thiol, NH, carboxylic acid (or salt thereof), phosphonic acid (or salt thereof) or phosphoric acid (or salt thereof) moiety of the drug;

wherein R⁶ is selected from the group consisting alkyl, substituted alkyl, aryl and substituted aryl; each R⁷ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Preferably Z′ is selected from the group consisting of a bond, —O—, —S—, —C(O)O—, OC(O)O—, —NR⁷C(O)O—, —OC(O)NR⁷—, —OP(O)(OR⁶)O—, —P(O)(OR⁶)O—, —NR⁷P(O)(OR⁶)O—, —C(O)NR⁷—, —NR⁷C(O)NR⁷—, —NR⁷C(O)NR⁷—, —S(O)₂NR⁷—, —S(O)—, —S(O)₂—, —C(O)S—, —ON═, —C(O)ON═, —NR⁷C(O)ON═, —C(O)OCR¹¹R¹²ON═, and a C═C linkage, wherein R⁶— R¹² are defined as above.

Preferably Y′ is a bond or a bivalent hydrocarbyl radical of 1 to 18 atoms having at least one alkylene, alkenylene or alkynylene group, with said at least one alkylene, alkenylene or alkynylene group optionally replaced with —O—, —S—, —NR⁷—, —C(O)—, —C(S)—, —OC(O)—, —C(O)O, —SC(O)—, —C(O)S—, —SC(S)—, —C(S)S—, —C(O)NR⁷—, —NR⁷C(O)—, arylene, substituted arylene, cycloalkylene, substituted cycloalkylene, cycloalkenylene, substituted cycloalkenylene, bivalent heterocyclic group or substituted bivalent heterocyclic group, where R⁷ is defined as above.

Y′ is also preferably represented by the formula:

(R³)_{f}(R⁴)_{g}(R⁵)ₕ

where each of R³, R⁴ and R⁵ are independently selected from the group consisting of alkylene, substituted alkylene, alkenylene, substituted alkenylene, alkynylene, substituted alkynylene, cycloalkylene, substituted cycloalkylene, cycloalkenylene, substituted cycloalkenylene, arylene, substituted arylene, heteroarylene, substituted heteroarylene, heterocyclene and substituted heterocyclene; and each of f, g and h are independently an integer from 0 to 3. More preferably, Y′ is alkylene, alkenylene or alkynylene.

Particularly preferred examples of suitable cleavable linkers Y′ for use in this invention include structures of formulae (i) through (v) as shown below;

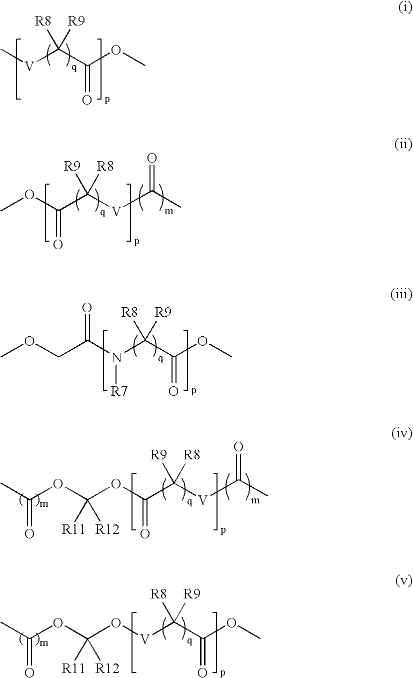

wherein V is selected from the group consisting of NR⁷, O, S and CR⁸R⁹; each m is independently 0 or 1; p is 0, 1, 2, 3 or 4; each q is 1, 2, 3, 4, 5 or 6; each R⁷, R⁸ and R⁹ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Another class of preferred compounds is represented by the formula (I-b):

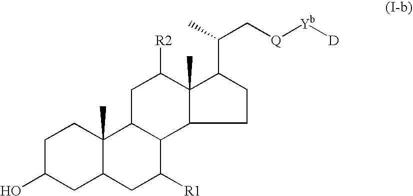

where Q is CH₂ or O;

Y^{b} is a cleavable linker;

D is a moiety derived from a drug;

R¹ is selected from the group consisting of hydrogen and OH;

R² is selected from the group consisting of hydrogen and OH.

wherein the compound of formula (I-b) above is a substrate for an intestinal bile acid transporter;

or pharmaceutically acceptable salts thereof.

The linker group, Y^{b}, is preferably from 1 to 20 atoms in length and Y^{b}-D together contain a moiety which is negatively charged at physiological pH. When drug D contains a primary or secondary amino group preferred compounds of formula (I-b) are represented by formulae (vi), (vii) and (viii) as shown below;

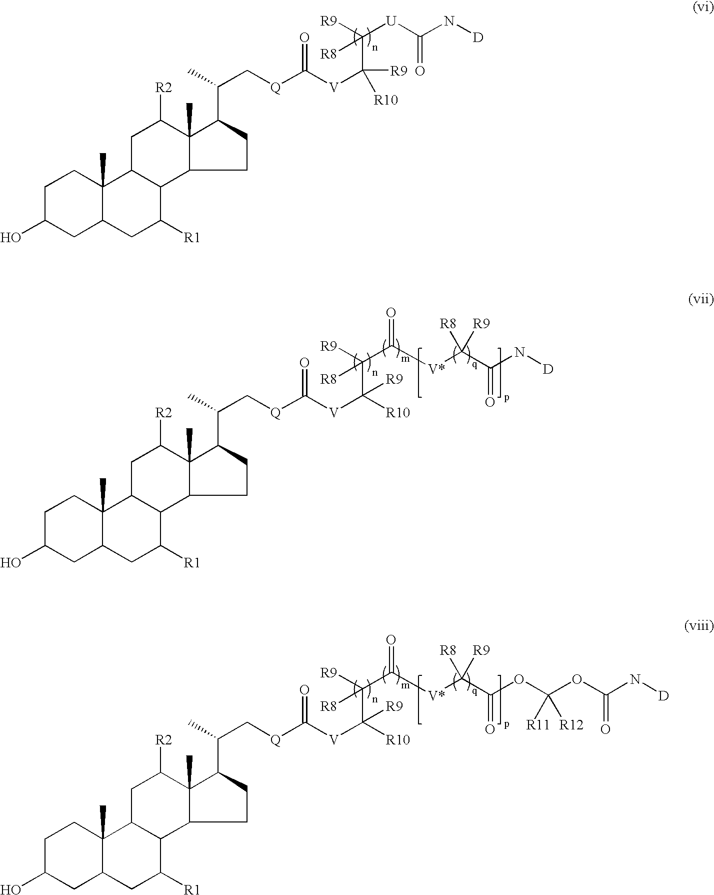

where Q is CH₂ or O; V and V* are independently NR⁷, O, S or CR⁸R⁹; U is NR⁷, O, S; R¹⁰ is R⁸ or (CR⁸R⁹)ᵣZ′; Z′ is selected from the group consisting of CO₂H, SO₃H, OSO₃H, SO₂H, P(O)(OR⁶)(OH), OP(O)(OR⁶)(OH) and pharmaceutically acceptable salts thereof; m is 0 or 1; n is 0, 1, 2, 3 or 4; p is 0, 1, 2, 3 or 4, providing that when m is 0 p is not 0; each q is 1, 2, 3, 4, 5 or 6; r is 0 or 1; R¹ is selected from the group consisting of hydrogen and OH; R² is selected from the group consisting of hydrogen and OH; R⁶ is selected from the group consisting of alkyl, substituted alkyl, aryl and substituted aryl; each R⁷, R⁸ and R⁹ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or when R⁷ and R⁹ are present and attached to adjacent atoms, then R⁷ and R⁹ together with the atoms to which they are attached form a cycloalkyl, substituted cycloalkyl, heterocycle or substituted heterocyclic ring; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

When drug D contains a hydroxyl group preferred compounds of formula (I-b) are represented by formulae (ix), (x) and (xi) as shown below;

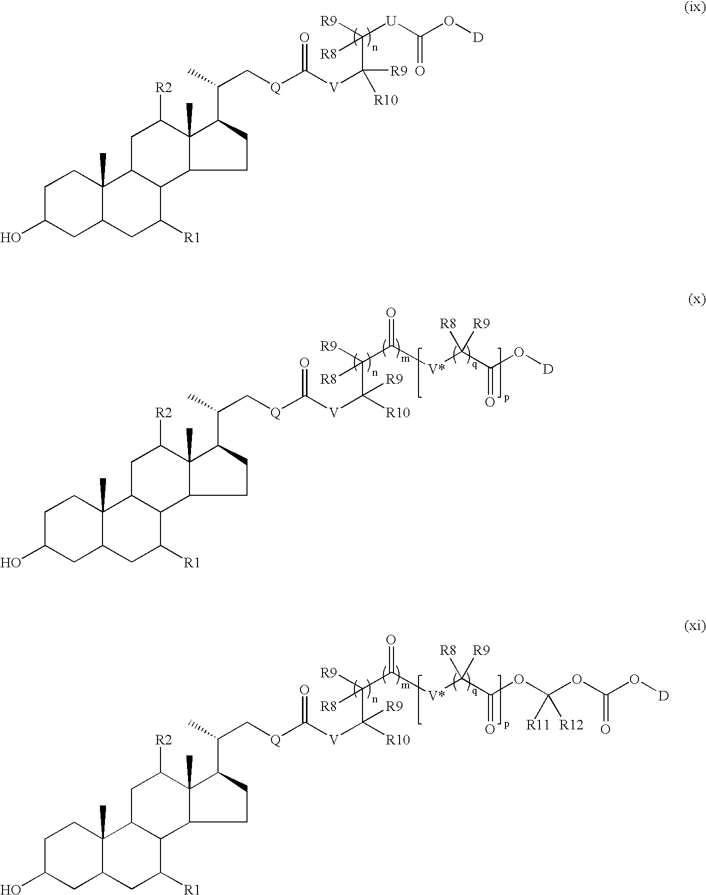

where Q is CH₂ or O; V and V* are independently NR⁷, O, S or CR⁸R⁹; U is NR⁷, O, S; R¹⁰ is R⁸ or (CR⁸R⁹)ᵣZ′; Z′ is selected from the group consisting of CO₂H, SO₃H, OSO₃H, SO₂H, P(O)(OR⁶)(OH), OP(O)(OR⁶)(OH) and pharmaceutically acceptable salts thereof; m is 0 or 1; n is 0, 1, 2, 3 or 4; p is 0, 1, 2, 3 or 4, providing that when m is 0 p is not 0; each q is 1, 2, 3, 4, 5 or 6; r is 0 or 1; R¹ is selected from the group consisting of hydrogen and OH; R² is selected from the group consisting of hydrogen and OH; R⁶ is selected from the group consisting of alkyl, substituted alkyl, aryl and substituted aryl; each R⁷, R⁸ and R⁹ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or when R⁷ and R⁹ are present and attached to adjacent atoms, then R⁷ and R⁹ together with the atoms to which they are attached form a cycloalkyl, substituted cycloalkyl, heterocycle or substituted heterocyclic ring; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

When drug D contains a carboxylic acid group preferred compounds of formula (I-b) are represented by formulae (xii) and (xiii) as shown below;

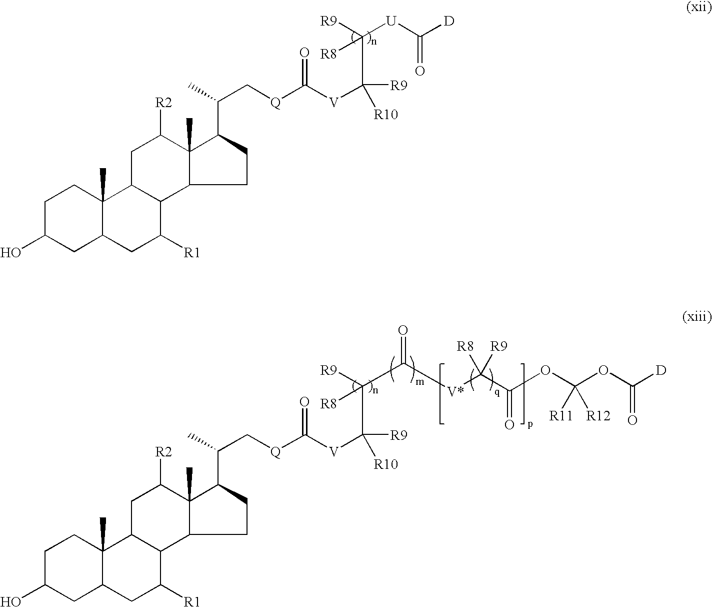

where Q is CH₂ or O; V and V* are independently NR⁷, O, S or CR⁸R⁹; U is NR⁷, O, S; R¹⁰ is R⁸ or (CR⁸R⁹)ᵣZ′; Z′ is selected from the group consisting of CO₂H, SO₃H, OSO₃H, SO₂H, P(O)(OR⁶)(OH), OP(O)(OR⁶)(OH) and pharmaceutically acceptable salts thereof; m is 0 or 1; n is 0, 1, 2, 3 or 4; p is 0, 1, 2, 3 or 4; each q is 1, 2, 3, 4, 5 or 6; r is 0 or 1; R¹ is selected from the group consisting of hydrogen and OH; R² is selected from the group consisting of hydrogen and OH; R⁶ is selected from the group consisting of alkyl, substituted alkyl, aryl and substituted aryl; each R⁷, R⁸ and R⁹ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or when R⁷ and R⁹ are present and attached to adjacent atoms, then R⁷ and R⁹ together with the atoms to which they are attached form a cycloalkyl, substituted cycloalkyl, heterocycle or substituted heterocyclic ring; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Another class of preferred compounds is represented by the formula (I-c):

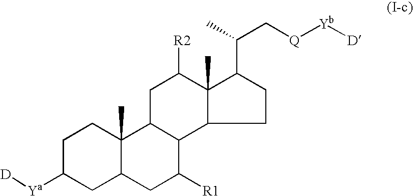

wherein:

Y^{a} is a cleavable linker;

Q is CH₂ or O;

Y^{b} is a cleavable linker;

D and D′ are moieties derived from drugs;

R¹ is selected from the group consisting of hydrogen and OH;

R² is selected from the group consisting of hydrogen and OH.

wherein the compound of formula (I-c) above is a substrate for an intestinal bile acid transporter;

or pharmaceutically acceptable salts thereof.

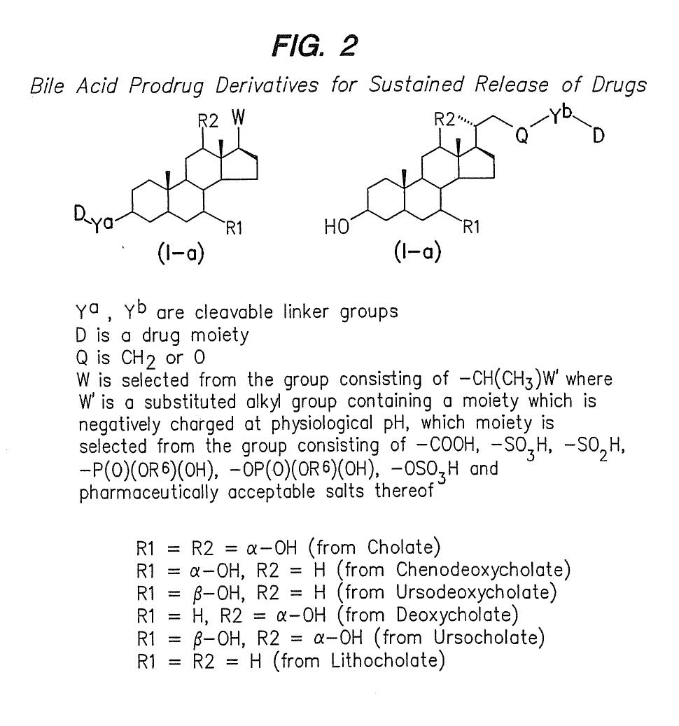

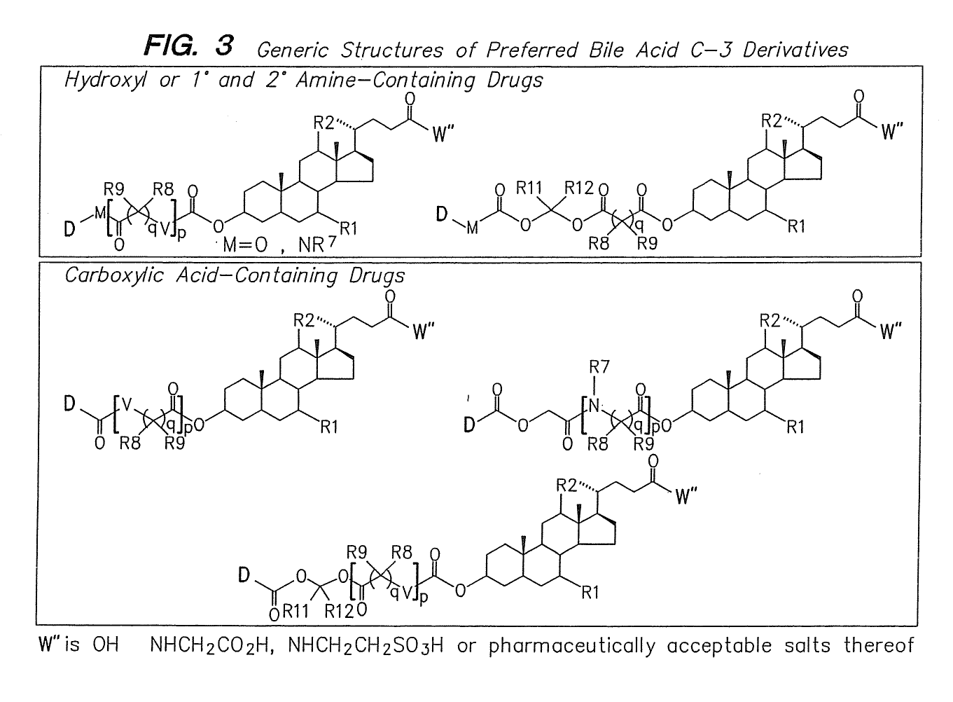

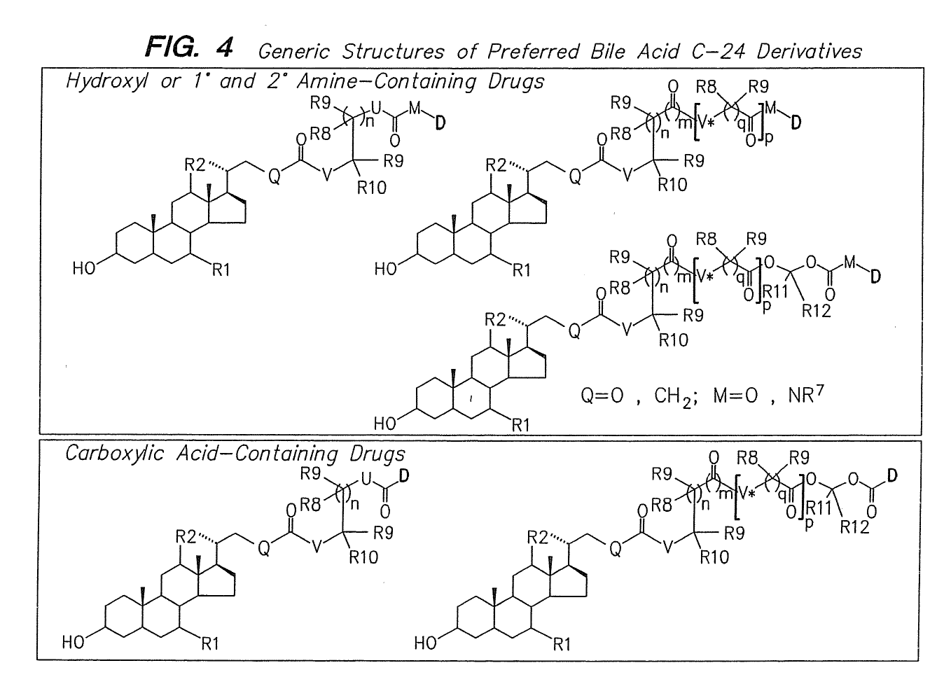

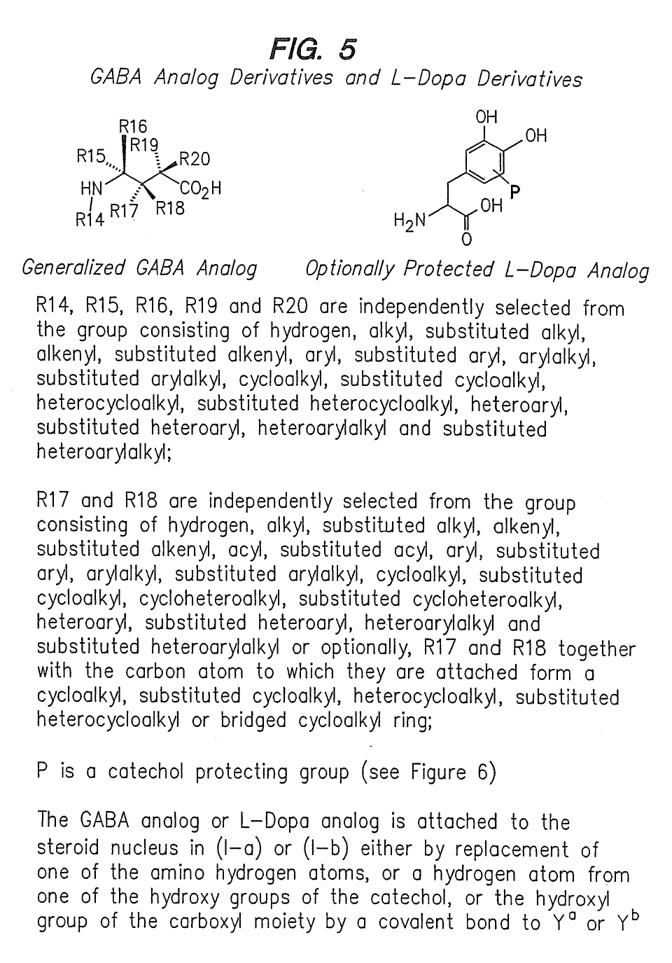

FIG. 2 illustrates further bile acid conjugates which can be used in a manner similar to those depicted above.

Note that the transporter proteins localized in intestinal epithelial cells and hepatocytes that are implicated in enterohepatic recycling of drugs and drug metabolites may come from different protein superfamilies. For example, the cholesterol lowering drug pravastatin is known to undergo enterohepatic recirculation, and in vitro transport studies using cloned rat transporters have demonstrated that pravastatin can serve as a substrate for an intestinal monocarboxylic acid transporter MCT, the sinusoidal hepatocyte transporter OATP2 and the canalicular hepatocyte transporter MRP2 (or cMOAT) (see Tokui et al, Pharm. Res. 1999, 16, 904-908; Yamazaki et al, Drug Metab. Dispos. 1997, 15, 1123-1129). In addition it is possible that transmembrane transport may be mediated by more than one mechanism. For example, active transport by multiple transporters within the membrane can contribute to transmembrane flux (e.g. sinusoidal uptake of glycocholate by NTCP and OATPs in hepatocytes), or alternatively both active pathways and passive diffusion may account for compound translocation.

Thus, in one embodiment of this invention, compounds of formula (I) are identified by in vitro screens that use cells expressing transporters selected from the plasma membranes of both intestinal epithelia and hepatocytes. Compounds of the formula D-Y-T that are found to be substrates for transporters from each of the 4 key membrane barriers (or are capable of passive diffusion across one or more of these barriers) will provide for sustained release of a drug D upon oral administration when a portion of the conjugate D-Y-T is cleaved during each cycle of the enterohepatic circulation. In practice it is often found that drugs have significantly greater permeability at the basolateral membrane of intestinal cells than at the apical membrane, making it less important to screen for transport at this basolateral membrane.

In a preferred embodiment, the moiety T is selected such that D-Y-T is a substrate for sinusoidal and canalicular hepatocyte anion transporters selected from the group consisting of OATs, OATPs, in particular OATP-C/LST-1, NTCP and MPR2, BSEP, MDR3 respectively. It is particularly preferred that the moiety T is further selected such that D-Y-T is also a substrate for one or more anion transporters in the apical membrane of intestinal epithelia, selected from the group consisting of the MCT's, OAT's, OATP's, SMVT, prostaglandin transporters, long chain fatty acid transporters, folate transporters and IBAT. In a second preferred embodiment, the moiety T is selected such that D-Y-T is a substrate for sinusoidal and canalicular hepatocyte anion transporters selected from the group consisting of OATs, OATPs, in particular OATP-C/LST-1, NTCP and MPR2, BSEP, MDR3 respectively. It is particularly preferred that the moiety T is further selected such that D-Y-T is also a substrate for one or more peptide transporters in the apical membrane of intestinal epithelia, selected from the group consisting of PEPT1 and PEPT2. In a third preferred embodiment, the moiety T is selected such that D-Y-T is a substrate for sinusoidal and canalicular hepatocyte cation transporters selected from the group consisting of the OCTs, MDR1 and related ABC binding cassette transporters. In this case it is particularly preferred that the moiety T is further selected such that D-Y-T is also a substrate for one or more cation transporters in the apical membrane of intestinal epithelia, selected from the group consisting of the OCTs, especially OCT1, OCTN1, OCTN2 and the polyamine transporters.

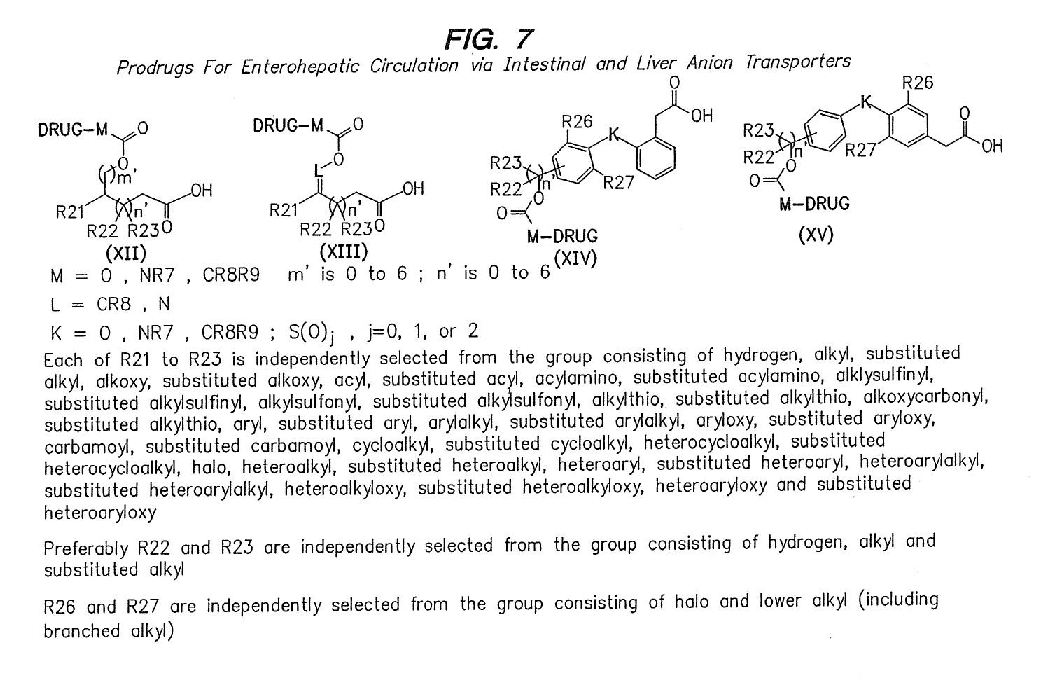

FIG. 7, for example, illustrates prodrugs for enterohepatic circulation and sustained release of the drug in vivo wherein the drug is conjugated to form compounds that are substrates for the intestinal and liver anion transporters. These prodrugs are formed from drugs that contain either one or more carboxylic acid, hydroxyl or primary or secondary amine moieties, and bear at least one carboxylic acid moiety that serves as a recognition element for anion transporters within the hepatocyte and intestinal cellular membranes.

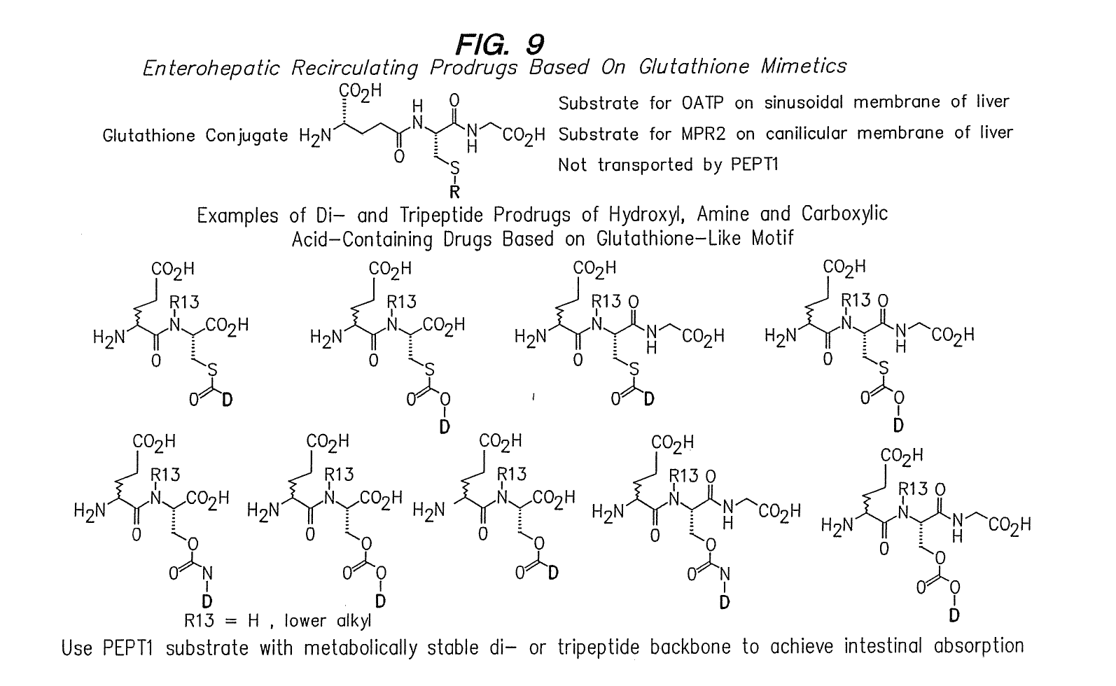

FIG. 8 depicts a strategy for achieving enterohepatic recycling of a prodrug or other compound by exploiting intestinal absorption by the peptide transporter, PEPT1, coupled with hepatic uptake and biliary secretion by anion transporters from the OATP and ABC transporter families respectively (e.g. specifically OATP1 and/or OATP2 in the sinusoidal membrane and MRP2 in the canalicular membrane of the hepatocyte). Prodrugs competent to undergo enterohepatic circulation via this mechanism are the glutathione mimetics illustrated in FIG. 9. Lipophilic glutathione conjugates like 2,4-dintrophenyl glutathione and leukotriene C₄ are known to be transported into the liver by the anion transporters oatp1 and OATP-C/LST-1/OATP2 and secreted into the bile via MRP2 (see Suzuki and Sugiyama, Sem. Liver Disease 2000, 20, 251-263; Kouzuki et al, J. Pharmacol. Exp. Ther. 1999, 288, 627-634; Gotoh et al, J. Pharmacol. Exp. Ther. 2000, 292, 433-439). While glutathione is not transported by PEPT1, its intestinal uptake is believed to occur (at least in part) via a yet uncharacterized glutathione transporter (e.g. see Iantomasi et al, Biochim. Biophys. Acta 1997, 1330, 274-283). The compounds in FIG. 9 are designed as PEPT1 substrates with a metabolically robust di- or tripeptide backbone (optionally achieved via incorporation of D-amino acids and N-alkyl amino acids) and a cleavable linker to a drug moiety that mimics the thiol derivatives naturally found in glutathione conjugates. These peptides undergo efficient oral absorption in mammals via uptake by PEPT1 in the intestine, and further undergo enterohepatic recirculation through the activity of anion transporters in the liver. The linker moiety within the prodrug (e.g. a thioester, ester, thiocarbonate, carbonate or carbamate bond) is slowly cleaved within the tissues of the enterohepatic circulation to release drug D into the systemic circulation. This provides for sustained release of the drug in vivo when compared to oral administration of the drug directly.

In addition to the complementary chemistry of the functional groups on the linker to the drug and transporter compound, the linker (when employed) is also selected to be cleavable in vivo. Cleavable linkers are well known in the art and are selected such that at least one of the covalent bonds of the linker that attaches the drug to the transporter compound can be broken in vivo thereby providing for the drug or active metabolite thereof to be available to the systemic blood circulation. The linker is selected such that the reactions required to break the cleavable covalent bond are favored at the physiological site in vivo which permits drug (or active metabolite thereof) release into the systemic blood circulation. The linker is further selected such that the rate of cleavage of the drug is controlled such that either only a portion of the total drug/cleavable linker/transporter is cleaved in each cycle through the enterohepatic circulation or that sustained release of the drug is achieved.

The selection of suitable cleavable linkers to provide effective concentrations of the drug or active metabolite thereof for release into the systemic blood circulation can be evaluated relative to one or more of the endogeneous enzymes of the enterohepatic circulation as set forth in the in vitro assay provided in Example 36 below. The use of such endogeneous enzymes in this in vitro assay provides a correlation to in vivo cleavage of the drug or active metabolite thereof from the drug/cleavable linker/transporter compound. The specific correlation of the in vitro results to in vivo results can be made by correlating in vivo concentrations of released drug as determined per Examples 38-42 below with the in vitro data. Again such correlation is well within the skill of the art.

Specifically, each candidate drug/cleavable linker/transporter compound is evaluated in this assay and the rate of cleavage of drug or active metabolite thereof from each candidate compound is determined. It is understood that the cleavage rate for each candidate compound will reflect several variables such as the specific drug employed, the chemistry and point of attachment of the drug to the cleavable linker, the specific linker employed, the chemistry and point of attachment of the transporter moiety to the cleavable linker, the enzyme or enzymes assayed, etc. While each of these factors plays a role in the rate of cleavage of drug or active metabolite thereof from the candidate compound, the overall effects of these factors and hence the release rate of the drug or active metabolite thereof can be routinely evaluated using the in vitro assay of Example 36.

The respective cleavage rates of candidate drug/cleavable linker/transporter compounds are then correlated to the desired cleavage rate for a particular drug such that sustained therapeutic and/or prophylactic concentrations of the drug or active metabolite thereof are provided to the systemic blood circulation. Such concentrations for each drug or active metabolite are readily ascertained by the skilled artisan using routine skill in the art based on the weight, age, sex, condition, etc. of the treated patient. In point of fact, for drugs that are currently delivered parenterally, intravenously, etc., such concentrations are already known in the art. Based on these factors, the skilled artisan can readily select the suitable drug/cleavable linker/transporter compound from the group of candidate compounds.

It is recognized that the exact cleavage mechanism employed is not critical to the methods of this invention provided, of course, that the drug/cleavable linker/transporter compound cleaves in vitro in some form to provide for the drug or active metabolite thereof for sustained release into the systemic blood circulation. For example, without being limited to any theory, several different cleavage scenarios are possible:

(a) after uptake across the apical enterocyte membrane, a portion of the drug/linker/transporter conjugate undergoes drug cleavage within the enterocyte and the liberated drug either diffuses passively across the basolateral membrane, or is subject to an active efflux process into the portal circulation. Drug that survives first pass hepatic extraction enters the systemic circulation while the remainder of the uncleaved drug/linker/transporter compound is subject to enterohepatic cycling and subsequent cleavage;

(b) the drug/linker/transporter conjugate is transported intact across the enterocyte and a portion undergoes cleavage in the portal blood. Drug that survives first pass hepatic extraction enters the systemic circulation while the remainder of the uncleaved drug/linker/transporter compound is subject to enterohepatic cycling and subsequent cleavage;

(c) the drug/linker/transporter conjugate is transported intact across the enterocyte and is extracted from the portal blood across the sinusoidal membrane of hepatocytes. Drug or active metabolite thereof resulting from cleavage of a portion of the pro-moiety within the hepatocyte may then rejoin the portal circulation via diffusion or active transport back across the basolateral membrane, while the remainder of the uncleared compound is subject to enterohepatic cycling and subsequent cleavage.

For drug/linker/transporter compounds, sustained release is achieved by controlled cleavage of the conjugate in any tissue that is encountered during enterohepatic circulation (e.g., contents of the intestinal lumen, enterocyte, blood, liver, biliary tract, etc). Preferred compounds of this invention are those wherein from about 1 to about 99% of the drug is cleaved during each cycle through the enterohepatic circulation and more preferably from about 5 to about 80%. The specific amount of drug released in each cycle can be preselected by use of an appropriate cleavable linker having well defined cleavage rates in vivo and is preferably selected relative to the drug delivered, the systemic concentration required in the patient treated which, of course, is dependent upon the age, weight and condition of the patient; all factors within the skill of the art.

When released at the levels set forth above, sustained release of the drug in vivo is achieved.

As noted above, the compounds of this invention are synthesized by conventional coupling reactions using as representative drugs, L-DOPA and gabapentin to cholic acid.

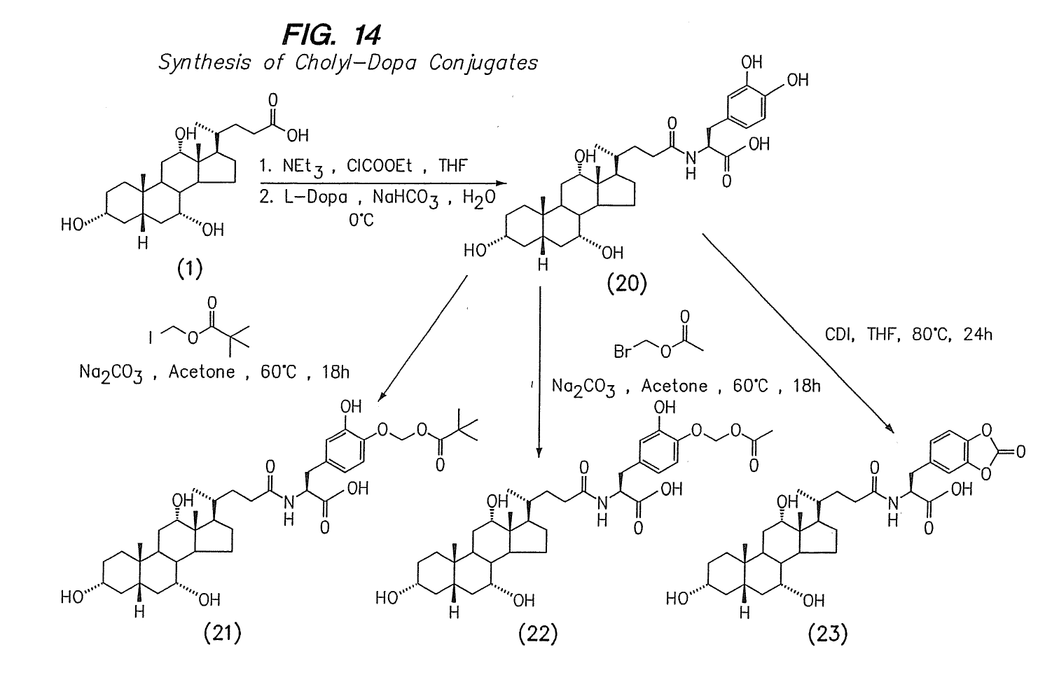

For example, there are several methods for the preparation of compounds where D is L-DOPA by relying on the amine group of L-DOPA to form an amide linkage by reaction of the C-24 carboxyl group of cholic acid with the amine group of L-DOPA suitably protected at one or both of the hydroxyl groups of the catechol moiety of L-DOPA. The methods in FIG. 14 involve the coupling of L-DOPA to cholic acid, (1), to form an amide linked derivative, (20). Subsequent protection of the catechol groups of derivative (20) leads to compounds (21), (22) and (23) as illustrated in the examples below.

Alternatively, the carboxyl group or the hydroxyl groups of L-DOPA can be used to effect coupling to cholic acid to provide for prodrugs of L-DOPA. Still further, the hydroxyl group as the 3-position of cholic acid can be used to effect compound coupling.

A more complete description of the synthesis of conjugates of L-DOPA or derivatives thereof with bile acids is provided in U.S. Provisional Patent Application Ser. No. 60/297,654 filed on Jun. 11, 2001 with Attorney Docket No. 033053-012 and entitled “BILE ACID PRODRUGS OF L-DOPA AND THEIR USE IN THE SUSTAINED TREATMENT OF PARKINSONISM” and U.S. patent application Ser. No. ______ filed concurrently herewith Attorney Docket No. 033053-028 and entitled “BILE ACID PRODRUGS OF L-DOPA AND THEIR USE IN THE SUSTAINED TREATMENT OF PARKINSONISM” which applications are incorporated herein by reference in their entirety.

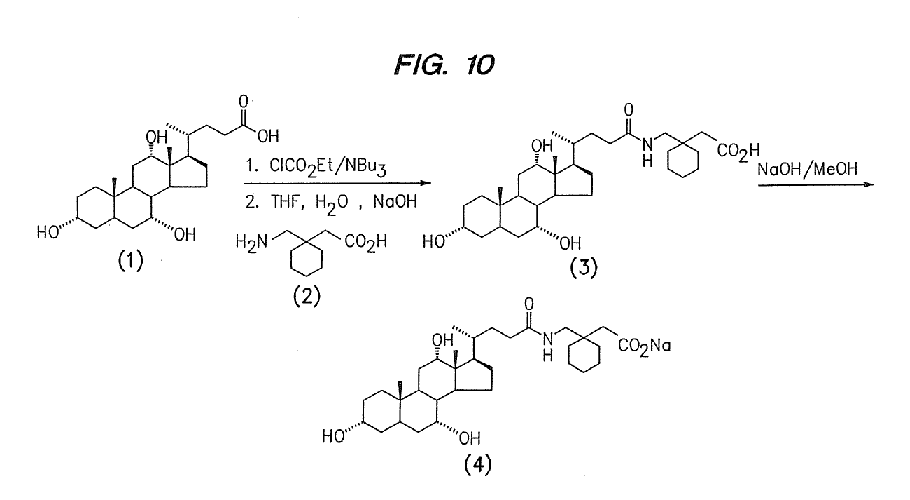

In another example, the drug, D, is gabapentin and, as before, there are several methods for the preparation of conjugates of this compound to a bile acid by relying on the amine group of gabapentin to form an amide linkage by reaction of the C-24 carboxyl group of cholic acid with the amine group of gabapentin. The methods in FIG. 10 involve the coupling of gabapentin, (2), to cholic acid, (1), to form an amide linked derivative, (3), which can be subsequently converted into a pharmaceutically acceptable sodium salt, (4), as illustrated in the examples below.

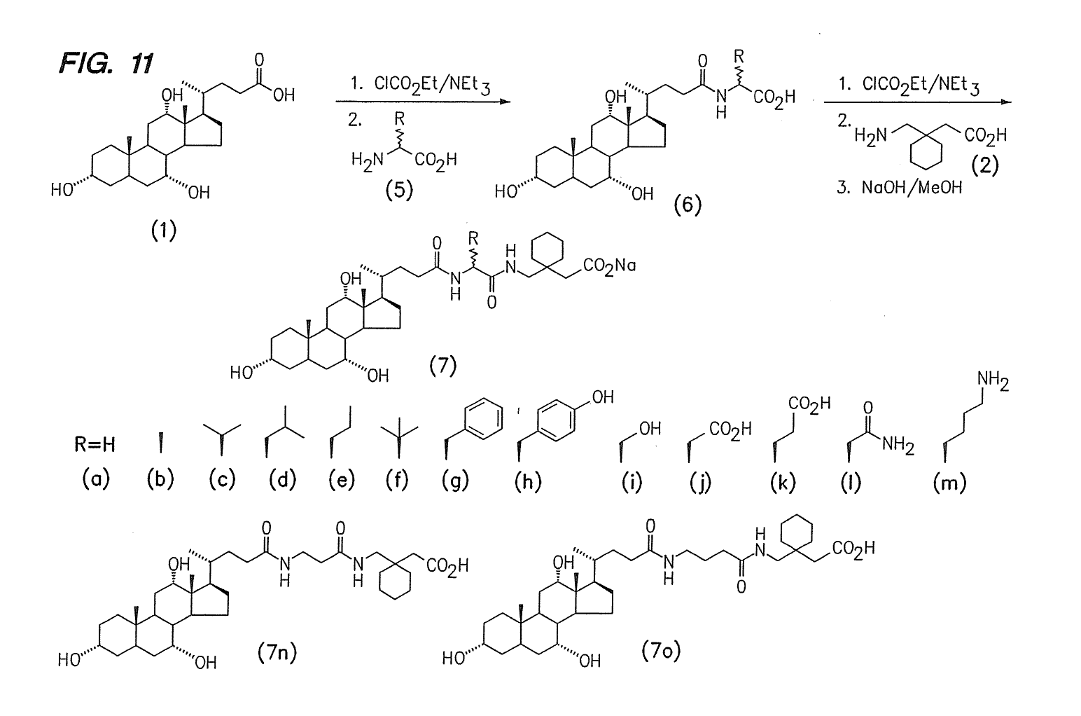

Alternatively, an amino acid linker group derived from amino acid, (5), can be employed to space gabapentin, (2), from cholic acid, (1), as illustrated in FIG. 11 and in the examples below. The amino acid linker group results in the formation of conjugates, (7), having a terminal dipeptide linked to the C-24 position of cholic acid. Conjugate (7) can participate in the bile acid transporter mechanism in vivo while the dipeptide fragment is a substrate for the PEPT1 and PEPT2 transporter mechanisms. The linking amino acid, (5), can be an α-amino acid, a β-amino acid or an omega amino acid.

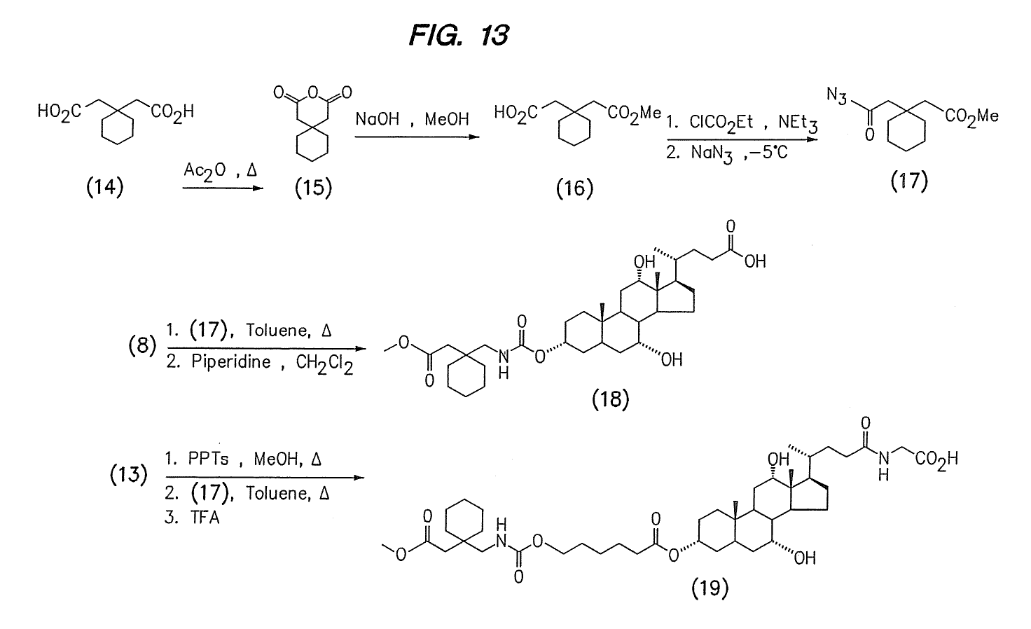

Still further, gabapentin can be conjugated to the 3-position of cholic acid as illustrated in FIGS. 2 and 13 to provide for compounds (18) and (19).

A more complete description of the synthesis of conjugates of gabapentin or derivatives thereof with bile acids or with amino acids is provided in U.S. Provisional Patent Application Ser. No. 60/297,472 filed on Jun. 11, 2001 with Attorney Docket No. 033053-013 and entitled “BILE ACID CONJUGATES FOR PROVIDING SUSTAINTED SYSTEMIC CONCENTRATIONS OF DRUGS”; U.S. patent application Ser. No. ______ filed concurrently herewith as Attorney Docket No. 033053-031 and entitled “BILE ACID CONJUGATES FOR PROVIDING SUSTAINTED SYSTEMIC CONCENTRATIONS OF DRUGS”; U.S. Provisional Patent Application Ser. No. 60/297,594 filed on Jun. 11, 2001 with Attorney Docket No. 033053-008 and entitled “BILE ACID CONJUGATES FOR PROVIDING SUSTAINTED SYSTEMIC CONCENTRATIONS OF DRUGS AFTER ORAL ADMINISTRATION”; U.S. patent application Ser. No. ______ filed concurrently herewith as Attorney Docket No. 033053-025 and entitled “BILE ACID CONJUGATES FOR PROVIDING SUSTAINTED SYSTEMIC CONCENTRATIONS OF DRUGS AFTER ORAL ADMINISTRATION”; U.S. Provisional Patent Application Ser. No. 60/297,732 filed on Jun. 11, 2001 with Attorney Docket No. 033053-016 and entitled “AMINO ACID CONJUGATES PROVIDING SUSTAINTED SYSTEMIC CONCENTRATIONS OF GABA ANALOGUES”; which applications are incorporated herein by reference in their entirety.

The drug, D, illustrated by L-DOPA and gabapentin above, can be any drug which can be conjugated to a transporter, preferably through a linker to form a conjugate which participates in the enterohepatic circulation of the animal.

As used herein, the term “drug” refers to a compound that exhibits therapeutic (i.e. therapeutic/prophylactic) or diagnostic utility when administered in effective amounts to a mammal. Preferably the drug exhibits therapeutic utility.

Preferably, the drug, D, has a carboxyl, an amino or a hydroxyl group for conjugation to the transporter to effect compounds having prolonged release in vivo.

Examples of drugs containing carboxyl groups include, for instance, angiotensin-converting enzyme inhibitors such as alecapril, captopril, 1-[4-carboxy-2-methyl-2R,4R-pentanoyl]-2,3-dihydro-2S-indole-2-carboxylic acid, enalaprilic acid, lisinopril, N-cyclopentyl-N-[3-[(2,2-dimethyl-1-oxopropyl)thio]-2-methyl-1-oxopropyl]glycine, pivopril, quinaprilat, (2R,4R)-2-hydroxyphenyl)-3-(3-mercaptopropionyl)-4-thiazolidinecarboxylic acid, (S) benzamido-4-oxo-6-phenylhexenoyl-2-carboxypyrrolidine, [2S-1[R*(R*))]]2α,3αβ,7αβ]-1[2-[[1-carboxy-3-phenylpropyl]-amino]-1-oxopropyl]octahydro-1H-indole-2-carboxylic acid, [3S-1-[R*(R*))]], 3R*]-2-[2-[[1-carboxy-3-phenylpropyl]-amino]-1-oxopropyl]-1,2,3,4-tetrahydro-3-isoquinolone carboxylic acid and tiopronin; cephalosporin antibiotics such as cefaclor, cefadroxil, cefamandole, cefatrizine, cefazedone, cefazuflur, cefazolin, cefbuperazone, cefixime, cefmenoxime, cefmetazole, cefodizime, cefonicid, cefoperazone, ceforanide, cefotaxime, cefotefan, cefotiam, cefoxitin, cefpimizole, cefpirome, cefpodoxime, cefroxadine, cefsulodin, cefpiramide, ceftazidime, ceftezole, ceftizoxime, ceftriaxone, cefuroxime, cephacetrile, cephalexin, cephaloglycin, cephaloridine, cephalosporin, cephanone, cephradine and latamoxef; penicillins such as amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperazillin, sulbenicllin, termocillin and ticarcillin; thrombin inhibitors such as argatroban, melagatran and napsagatran; influenza neuraminidase inhibitors such as zanamivir and BCX-1812; non-steroidal antiinflammatory agents such as acametacin, alclofenac, alminoprofen, aspirin (acetylsalicylic acid), 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tolmetin and zopemirac; prostaglandins such as ciprostene, 16-deoxy-16-hydroxy-16-vinyl prostaglandin E₂, 6,16-dimethylprostaglanidin E₂, epoprostostenol, meteneprost, nileprost, prostacyclin, prostaglandins E₁, E₂, or F_{2α} and thromboxane A₂; quinolone antibiotics such as acrosoxacin, cinoxacin, ciprofloxacin, enoxacin, flumequine, naladixic acid, norfiloxacin, ofloxacin, oxolinic acid, pefloxacin, pipemidic acid and piromidic acid; other antibiotics such as aztreonam, imipenem, meropenem and related carbopenem antibiotics.

Representative drugs containing amine groups include: acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, a minorex, aprinocid, cambendazole, cimetidine, cisapride, clonidine, cyclobenzadole, delavirdine, efegatrin, etintidine, fenbendazole, fenmetazole, flubendazole, fludorex, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, procainamide, ranitidine, tetrahydrazoline, tiamenidine, tinazoline, tiotidine, tocamide, tolazoline, tramazoline, xylometazoline, dimethoxyphenethylamine, N-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, clorprenaline, chlortermine, dopamine, ephrinephrine etryptamine, fenfluramine, methyldopamine, norepinephrine, tocamide, enviroxime, nifedipine, nimodipine, triamterene, norfloxacin and similar compounds such as pipedemic acid, 1-ethyl-6-fluoro-1,4-dihydro-4-oxo-7-(1-piperazinyl)-1,8-napthyridine-3-carboxylic acid, 1-cyclopropyl-6-fluoro-1,4-dihydro-4-oxo-7-(piperazinyl)-3-quinolinecarboxylic acid.

Representative drugs containing hydroxy groups include: steroidal hormones such as allylestrenol, cingestol, dehydroepiandrosteron, dienostrol, diethylstilbestrol, dimethisteron, ethyneron, ethynodiol, estradiol, estron, ethinyl estradiol, ethisteron, lynestrenol, mestranol, methyl testosterone, norethindron, norgestrel, norvinsteron, oxogeston, quinestrol, testosteron and tigestol; tranquilizers such as dofexazepain, hydroxyzin, lorazepam and oxazepam; neuroleptics such as acetophenazine, carphenazine, fluphenazine, perphenyzine and piperaetazine; cytostatics such as aclarubicin, cytarabine, decitabine, daunorubicin, dihydro-5-azacytidine, doxorubicin, epirubicin, estramustin, etoposide, fludarabine, gemcitabine, 7-hydroxychlorpromazin, nelarabine, neplanocin A, pentostatin, podophyllotoxin, tezacitabine, troxacitabine, vinblastin, vincristin, vindesin; hormones and hormone antagonists such as buserilin, gonadoliberin, icatibrant and leuprorelin acetate; antihistamines such as terphenadine; analgesics such as diflunisal, naproxol, paracetamol, salicylamide and salicyclic acid; antibiotics such as azidamphenicol, azithromycin, camptothecin, cefamandol, chloramphenicol, clarithromycin, clavulanic acid, clindamycin, demeclocyclin, doxycyclin, erythromycin, gentamycin, imipenem, latamoxef, metronidazole, neomycin, novobiocin, oleandomycin, oxytetracyclin, tetracycline, thiamenicol and tobramycin; antivirals such as acyclovir, d4C, ddC, DMDC, Fd4C, FddC, FMAU, FTC, 2′-fluoro-ara-dideoxyinosine, ganciclovir, lamivudine, penciclovir, SddC, stavudine, 5-trifluoromethyl-2′-deoxyuridine, zalcitabine and zidovudine; bisphosphonates such as EB-1053, etidronate, ibandronate, olpadronate, residronate, YH-529 and zolendronate; protease inhibitors such as ciprokiren, enalkiren, ritonavir, saquinavir and terlakiren; prostaglandins such as arbaprostil, carboprost, misoprostil and prostacydin; antidepressives such as 8-hydroxychlorimipramine and 2-hydroxyimipramine; antihypertonics such as sotarol and fenoldopam; anticholinerogenics such as piperidine, procyclidin and trihexyphenidal; antiallergenics such as cromolyn; glucocorticoids such as betamethasone, budenosid, chlorprednison, clobetasol, clobetasone, corticosteron, cortisone, cortodexon, dexamethason, flucortolon, fludrocortisone, flumethasone, flunisolid, fluprednisolon, flurandrenolide, flurandrenolon acetonide, hydrocortisone, meprednisone, methylpresnisolon, paramethasone, prednisolon, prednisol, triamcinolon and triamcinolon acetonide; narcotic agonists and antagonists such as apomorphine, buprenorphine, butorphanol, codein, cyclazocin, hydromorphon, ketobemidon, levallorphan, levorphanol, metazocin, morphine, nalbuphin, nalmefen, naloxon, nalorphine, naltrexon, oxycodon, oxymorphon and pentazocin; stimulants such asmazindol and pseudoephidrine; anaesthetics such as hydroxydion and propofol; β-receptor blockers such as acebutolol, albuterol, alprenolol, atenolol, betazolol, bucindolol, cartelolol, celiprolol, cetainolol, labetalol, levobunelol, metoprolol, metipranolol, nadolol, oxyprenolol, pindolol, propanolol and timolol; α-sympathomimetics such as adrenalin, metaraminol, midodrin, norfenefrin, octapamine, oxedrin, oxilofrin, oximetazolin and phenylefrin; β-sympathomimetics such as bamethan, clenbuterol, fenoterol, hexoprenalin, isoprenalin, isoxsuprin, orciprenalin, reproterol, salbutamol and terbutalin; bronchodilators such as carbuterol, dyphillin, etophyllin, fenoterol, pirbuterol, rimiterol and terbutalin; cardiotonics such as digitoxin, dobutamin, etilefrin and prenalterol; antimycotics such as amphotericin B, chlorphenesin, nystatin and perimycin; anticoagulants such as acenocoumarol, dicoumarol, phenprocoumon and warfarin; vasodilators such as bamethan, dipyrimadol, diprophyllin, isoxsuprin, vincamin and xantinol nicotinate; antihypocholesteremics such as compactin, eptastatin, mevinolin and simvastatin; miscellaneous drugs such as bromperidol (antipsychotic), dithranol (psoriasis) ergotamine (migraine) ivermectin (antihelminthic), metronidazole and secnizadole (antiprotozoals), nandrolon (anabolic), propafenon and quinadine (antiarythmics), quetiapine (CNS), serotonin (neurotransmitter) and silybin (hepatic disturbance).

Preferably the drug is not a GABA analog; L-Dopa, an L-aromatic amino acid decarboxylase inhibitor, or catechol O-methyl transferase inhibitor or derivatives thereof; a naturally occurring α-amino acid or an ester or carboxamide of a naturally occurring α-amino acid; a polypeptide or peptidomimetic derived from a linear oligopeptide containing at least 3 amino acids; an oligonucleotide; a cyclophane derivative, a diethylenetriaminopentaacetate derivative, or paramagnetic ion chelates thereof; histamine or tyramine; 5-de-O-methylsporaricin; a bis-(2-chloroethyl)amine containing nitrogen mustard; an HMG-CoA reductase inhibitor; a proline hydroxylase inhibitor; fluvalinate; a steroid containing the carbon substructures of the following formula:

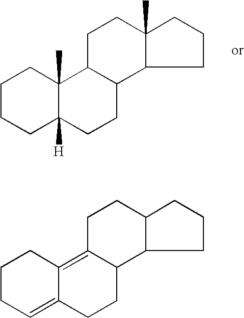

FIG. 7 illustrates compounds which do not employ bile acids to form drug conjugates which participate in the enterohepatic circulation but, rather, employ compounds which are substrates for the intestinal and liver anion transporters. The conjugates depicted can be readily synthesized by any known means including those illustrated above.

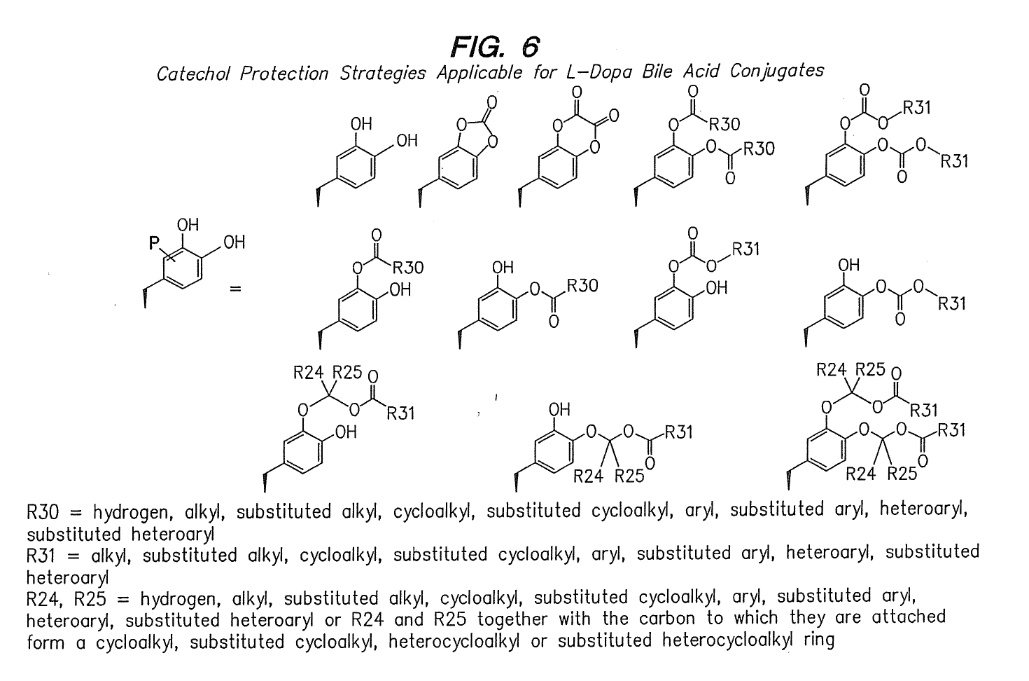

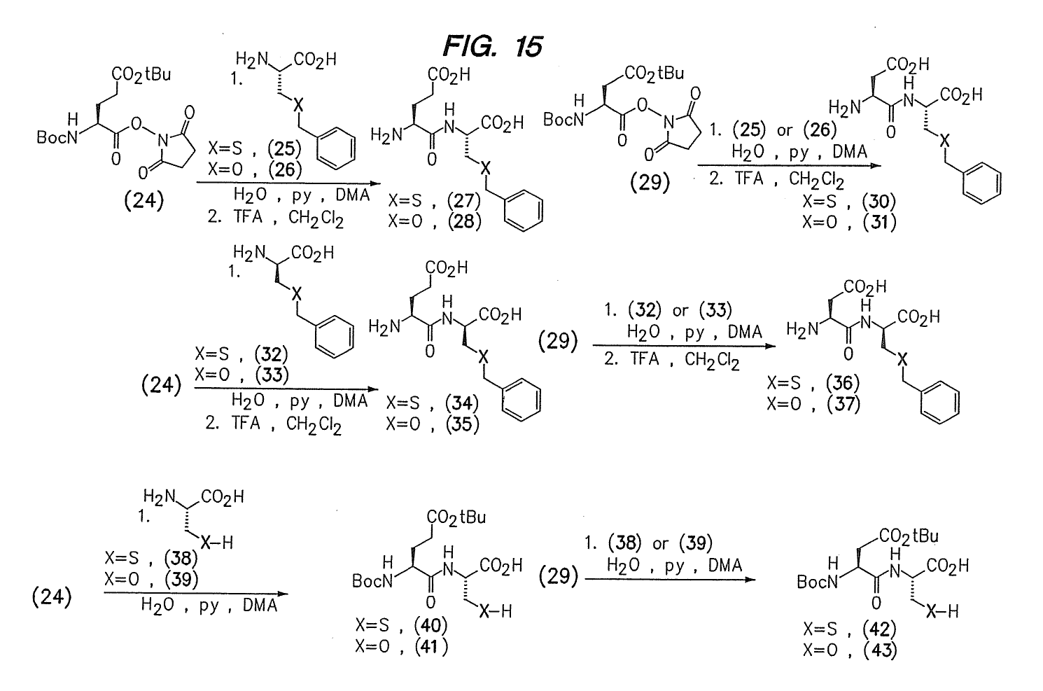

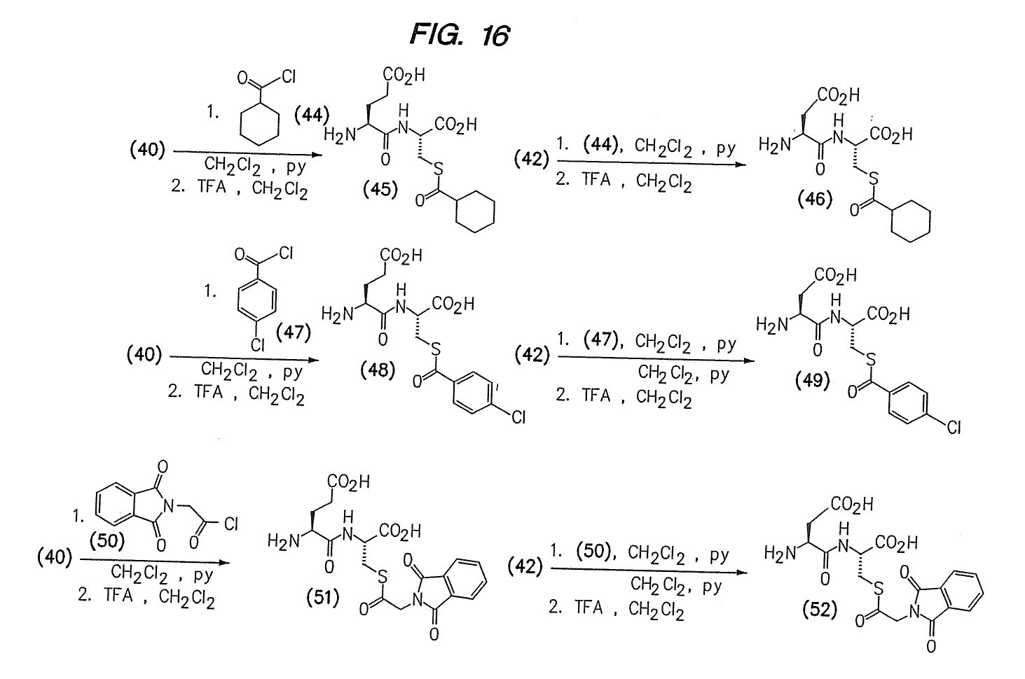

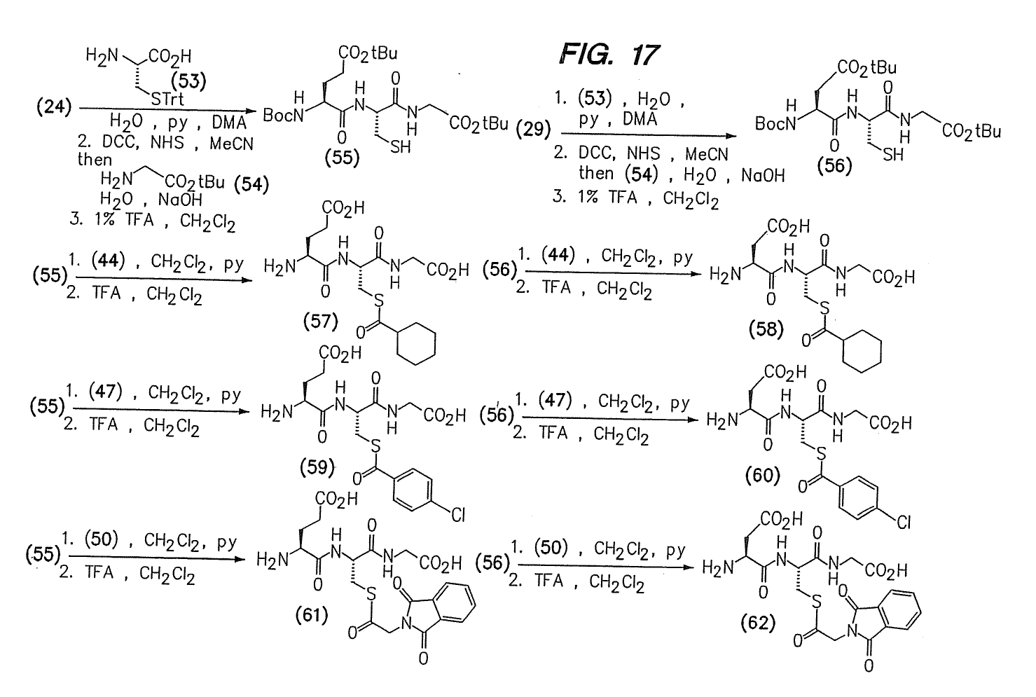

FIGS. 15-17 illustrate the synthesis of several glutathione mimetic conjugates capable of undergoing enterohepatic circulation via the intestinal peptide transporter and liver anion transporters. As demonstrated in Example 34 below, compounds (27), (28) and (30) derived from either L-Cysteine or L-Serine are efficient substrates for hPEPT1 while compounds (34)-(37) derived from D-Cysteine and D-Serine are not transported by hPEPT1. While these compounds are not prodrugs, the related adducts of the lipophilic acids cyclohexane carboxylic acid, 4-chlorobenzoic acid and N-phthaloylglycine shown in FIGS. 6 and 17 are effective prodrugs that undergo enterohepatic recirculation in vivo. N-Phthaloylglycine is a potential anticonvuslant agent whose efficacy is compromised via very rapid clearance (half-life ˜10 minutes in rats, see Salach et al, Pharm. Res. 1994, 11, 1429-1434). Compound (61) is a prodrug of N-phthaloylglycine that undergoes enterohepatic circulation and cleavage in vivo to provide sustained exposure to the drug, as illustrated in Example 42 below.

### Utility

The compounds and methods described herein permit sustained release of the drug or active metabolite thereof relative to oral dosing with the parent drug itself.

### Pharmaceutical Formulations

When employed as pharmaceuticals, the compounds of this invention are usually administered in the form of pharmaceutical compositions that are administered by oral routes. Such compositions are prepared in a manner well known in the pharmaceutical art and comprise at least one active compound.

This invention also includes pharmaceutical compositions that contain, as the active ingredient, one or more of the compounds of this invention associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, etc. containing, for example, up to 90% by weight of the active compound using, for example, soft and hard gelatin capsules.

In preparing a formulation, it may be necessary to mill the active compound to provide the appropriate particle size prior to combining with other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. ˜40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 1 mg to about 6 g of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 2 g of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

The following synthetic and biological examples are offered to illustrate this invention and are not to be construed in any way as limiting the scope of this invention. Unless otherwise stated, all temperatures are in degrees Celsius.

### EXAMPLES

In the examples below, the following abbreviations have the following meanings. If an abbreviation is not defined, it has its generally accepted meaning.
ATCC=American Type Tissue Culture
Atm=atmosphere
Boc=tert-butyloxycarbonyl
Cbz=carbobenzyloxy
CHO=Chinese hampster ovary
CPM=counts per minute
DIC=diisopropylcarbodiimide
DMAP=4-N,N-dimethylaminopyridine
DMEM=Dulbecco's minimum eagle medium
DMF=N,N-dimethylformamide
DMSO=dimethylsulfoxide
EDTA=ethylene diamine tetraacetic acid
FMOC=9-fluorenylmethyloxycarbonyl
g=gram
GDC=glycodeoxycholate
GTP=guanosine 5′-triphosphate
h=hour
HBSS=Hank's buffered saline solution
Hz=hertz
kg=kilogram
EBAT=intestinal bile acid transporter
L=liter
LBAT=liver bile acid transporter
LC/MS=liquid chromatography/mass spectroscopy
mg=milligram
mL=milliLiter
mmol=millimol
mm=millimeter
mM=millimolar
min.=minute
MRM=multiple reaction monitoring
MS=mass spectroscopy
mV=millivolts
mΩ=milliohms
NTCP=Na+ taurocholate cotransporting polypeptide
PBS=phosphate buffered saline
PPTS=pyridinium p-toluenesulfonate
PEG400 polyethylene glycol 400
Penstrep=penicillin/streptomycin
THF=tetrahydrofuran
TFA=trifluoroacetic acid
TLC=thin layer chromatography
TMSOTf=trimethylsilyltrifluoromethane-sulfonate
Trisyl=2,4,6-triispropylbenzenesulfonyl
μA=microamperes
μg=microgram
μL=microliter
μM=micromolar
μm=micron
v/v=volume to volume

### Experimental Methods

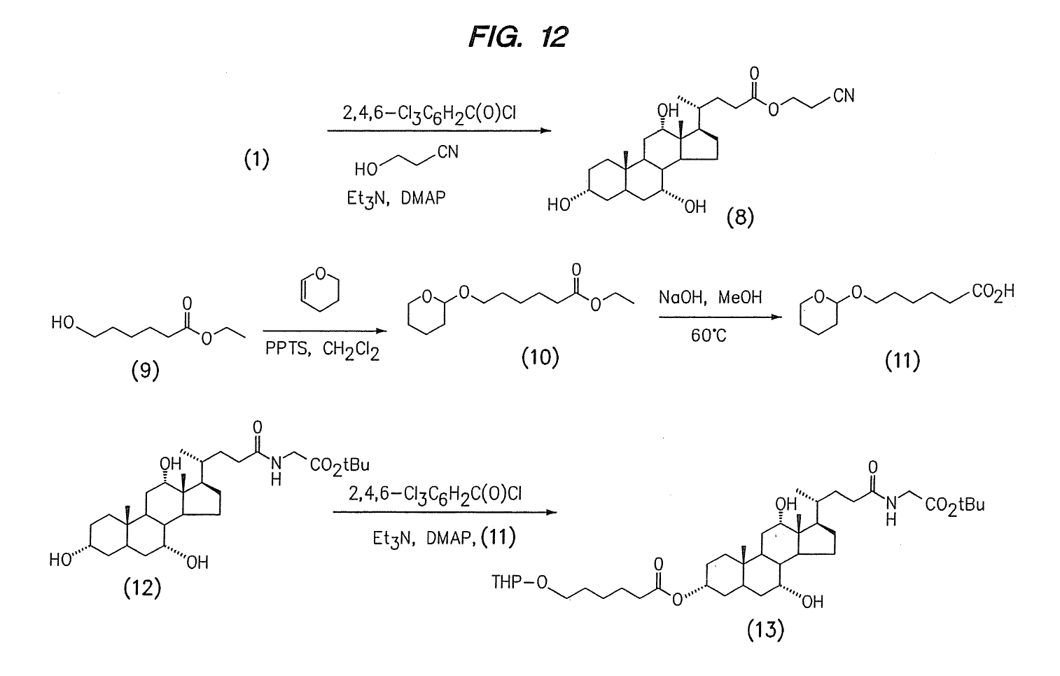

The following examples illustrate how the synthesis of drug/linker/transporter conjugates could be conducted in order to prepare compounds of formula (I). The syntheses described below are illustrated in FIGS. 10-17.

### Example 1

### Synthesis of Compound (4)

Cholic acid (1) (408 mg, 1 mmol) was dissolved in anhydrous THF (10 mL) and tributylamine (0.285 mL, 1.2 mmol) added slowly with stirring. The solution was cooled to −5° C. in an ice-salt bath, and ethyl chloroformate (0.12 mL, 1.2 mmol) added slowly, maintaining the temperature between −5 to 0° C. After addition was complete, the cold mixture was stirred for an additional 15 minutes. A solution containing 1-aminomethyl-1-cyclohexaneacetic acid hydrochloride (Gabapentin, RBI Sigma) (2) (363 mg, 1.75 mmol) in 2N NaOH (3 mL) was added and the mixture stirred for an additional 60 min at −5 to 0° C. After removal of the THF in vacuo, saturated NaHCO₃ (15 mL) was added and the aqueous mixture washed with EtOAc (3×10 mL), then the pH adjusted to 3-4 with citric acid. The product was extracted into EtOAc (3×15 mL), and the combined organic phase dried over MgSO₄, and concentrated to dryness. The residue was purified by flash chromatography on silica gel (5% MeOH/CH₂Cl₂) to give pure free acid (3) (287 mg, 52% yield). Electrospray mass spectrometry showed the expected molecular ion at m/z=562.6 (M+H⁺). The corresponding sodium salt (4) was prepared in quantitative yield from (3) (287 mg, 0.52 mmol) by addition of a methanol solution of (3) to water containing 0.5N NaOH (1 eq.) and evaporation to dryness on a lyophilizer.

MS (ESI): m/z=560.6 (M-Na⁻).

¹H NMR (CD₃OD, 400 MHz, characteristic resonances only): 3.34 (s, 2H), 2.28 (s, 2H), 1.03 (d, 3H, J=6.4 Hz), 0.91 (s, 3H), 0.70 (s, 3H).

### Example 2

### Synthesis of Cholic Acid Gabapentin Dipeptides (7)

Cholic acid (1) (408 mg, 1 mmol) was dissolved in anhydrous THF (10 mL) and triethylamine (0.167 mL, 1.2 mmol) added slowly with stirring. The solution was cooled to −5° C. in an ice-salt bath, and ethyl chloroformate (0.12 mL, 1.2 mmol) added slowly, maintaining the temperature between −5 to 0° C. After addition was complete, the cold mixture was stirred for an additional 15 minutes. A solution containing an amino acid (5) (1.75 mmol) in 2N NaOH (2 mL) was added and the mixture stirred for an additional 60 min at −5 to 0° C. After removal of the THF in vacuo, saturated NaHCO₃ (15 mL) was added and the aqueous mixture washed with EtOAc (3×10 mL), then the pH adjusted to 3-4 with citric acid. The product was extracted into EtOAc (3×15 mL), and the combined organic phase dried over MgSO₄, and concentrated to dryness. The residue was purified by flash chromatography on silica gel (5% MeOH/CH₂Cl₂) to give the pure cholic acid adduct (6). This compound (0.4 mmol) was dissolved in anhydrous THF (10 mL) and triethylamine (0.44 mmol) added slowly with stirring. The solution was cooled to −5° C. in an ice-salt bath, and ethyl chloroformate (44 μL, 0.44 mmol) added slowly, maintaining the temperature between −5 to 0° C. After addition was complete, the cold mixture was stirred for an additional 15 minutes. A solution containing gabapentin (2) (166 mg, 0.8 mmol) in 2N NaOH (3 mL) was added and the mixture stirred for an additional 60 min at −5 to 0° C. After removal of the THF in vacuo, saturated NaHCO₃ (10 mL) was added and the aqueous mixture washed with EtOAc (3×10 mL), then the pH adjusted to 3-4 with citric acid. The product was extracted into EtOAc (3×20 mL), and the combined organic phases dried over MgSO₄, and concentrated to dryness. The residue was purified by flash chromatography on silica gel (10% MeOH/CH₂Cl₂) to give the pure cholic acid gabapentin dipeptide derivative. The corresponding sodium salt (7) was prepared in quantitative yield by addition of a methanol solution of the acid to water containing 0.5N NaOH (1 eq.) and evaporation to dryness on a lyophilizer. Compounds were characterized by electrospray mass spectrometry as reported below:

Cholyl-Gly-Gabapentin (7a): MS (ESI): m/z 617.50 (M−H⁻), 619.51 (M+H⁺).

¹H NMR (CD₃OD, 400 MHz, characteristic resonances only): 3.81 (s, 2H), 3.34 (s, 2H), 2.28 (s, 2H), 1.03 (d, 3H, J=6.4 Hz), 0.91 (s, 3H), 0.70 (s, 3H).

Cholyl-Ala-Gabapentin (7b): MS (ESI): m/z 631.50 (M−H⁻), 633.52 (M+H⁺).

¹H NMR (CD₃OD, 400 MHz, characteristic resonances only): 4.29 (m, 1H), 3.34 (s, 2H), 2.28 (s, 2H), 1.34 (d, 2H, J=6.8 Hz), 1.01 (d, 3H, J=6.4 Hz), 0.91 (s, 3H), 0.71 (s, 3H).

Cholyl-Val-Gabapentin (7c): MS (ESI): m/z 659.55 (M−H⁻), 661.55 (M+H⁺).

¹H NMR (CD₃OD, 400 MHz, characteristic resonances only): 4.26 (m, 1H), 3.34 (s, 2H), 2.27 (s, 2H), 1.02 (d, 3H, J=6.4 Hz), 0.97 (d, 6H, J=6.4 Hz), 0.91 (s, 3H), 0.71 (s, 3H).

Cholyl-Leu-Gabapentin (7d): MS (ESI): m/z 673.43 (M−H⁻), 675.45 (M+H⁺).

¹H NMR (CD₃OD, 400 MHz, characteristic resonances only): 4.34 (m, 1H), 3.34 (s, 2H), 2.27 (s, 2H), 1.02 (d, 3H, J=6.4 Hz), 0.97 (d, 3H, J=6.4 Hz), 0.92 (d, 3H, J=6.4 Hz), 0.91 (s, 3H), 0.70 (s, 3H).

Cholyl-Norleu-Gabapentin (7e): MS (ESI): m/z 659.56 (M−H⁻), 661.57 (M+H⁺).

¹H NMR (CD₃OD, 400 MHz, characteristic resonances only): 4.26 (m, 1H), 3.34 (s, 2H), 2.27 (s, 2H), 1.02 (d, 3H, J=6.4 Hz), 0.91 (s, 3H), 0.71 (s, 3H).

Cholyl-^{t}BuGly-Gabapentin (7f): MS (ESI): m/z 673.58 (M−H⁻), 675.58 (M+H⁺).

¹H NMR (CD₃OD, 400 MHz, characteristic resonances only): 4.20 (s, 1H), 3.34 (s, 2H), 2.29 (s, 2H), 1.01 (d, 3H, J=6.4 Hz), 0.98 (s, 9H), 0.91 (s, 3H), 0.70 (s, 3H).

Cholyl-Phe-Gabapentin (7g): MS (ESI): m/z 707.47 (M−H⁻), 709.36 (M+H⁺).

¹H NMR (CD₃OD, 400 MHz, characteristic resonances only): 7.26 (m, 5H), 4.59 (m, 1H), 3.34 (s, 2H), 3.25-2.95 (m, 2H), 2.18 (d, 2H, J=7.2 Hz), 0.98 (d, 3H, J=6.4 Hz), 0.91 (s, 3H), 0.68 (s, 3H).

Cholyl-Tyr-Gabapentin (7h): MS (ESI): m/z 723.42 (M−H⁻), 725.42 (M+H⁺).

¹H NMR (CD₃OD, 400 MHz, characteristic resonances only): 7.06 (d, 2H, J=8.8 Hz), 6.69 (d, 2H, J=8.8 Hz), 4.51 (dd, 1H, J=6.8 Hz, J=8.8 Hz), 3.34 (s, 2H), 3.16-2.78 (m, 2H), 2.16 (d, 2H, J=7.2 Hz), 0.98 (d, 3H, J=6.4 Hz), 091 (s, 3H), 0.68 (s, 3H).

Cholyl-Ser-Gabapentin (71): MS (ESI): m/z 647.42 (M−H⁻), 649.41 (M+H⁺).

¹H NMR (CD₃OD, 400 MHz, characteristic resonances only): 4.37 (m, 1H), 3.78 (m, 2H), 3.34 (s, 2H), 2.15 (s, 2H), 1.03 (d, 3H, J=6.4 Hz), 0.91 (s, 3H), 0.71 (s, 3H).

Cholyl-Asp-Gabapentin (7j): MS (ESI): m/z 647.45 (M−H⁻), 649.45 (M+H⁺).

¹H NMR (CD₃OD, 400 MHz, characteristic resonances only): 4.71 (m, 1H), 3.34 (s, 2H), 2.87-2.65 (m, 2H), 2.28 (s, 2H), 1.02 (d, 3H, J=6.4 Hz), 0.91 (s, 3H), 0.71 (s, 3H).

Cholyl-Glu-Gabapentin (7k): MS (ESI): m/z 688.50 (M−H⁻), 690.54 (M+H⁺).

¹H NMR (CD₃OD, 400 MHz, characteristic resonances only): 4.35 (m, 1H), 3.34 (s, 2H), 2.38 (t, 2H, J=7 Hz), 1.02 (d, 3H, J=6.4 Hz), 0.91 (s, 3H), 0.71 (s, 3H).

Cholyl-Asn-Gabapentin (71): MS (ESI): m/z 674.43 (M−H⁻), 676.44 (M+H⁺).

¹H NMR (CD₃OD, 400 MHz, characteristic resonances only): 4.29 (m, 1H), 3.34 (s, 2H), 2.92-2.69 (m, 2H), 2.28 (s, 2H), 1.02 (d, 3H, J=6.4 Hz), 0.91 (s, 3H), 0.70 (s, 3H).

Cholyl-Lys-Gabapentin (7m): MS (ESI): m/z 688.50 (M−H⁻), 690.54 (M+H⁺)<

¹H NMR (CD₃OD, 400 MHz, characteristic resonances only): 4.29 (m, 1H), 3.34 (s, 2H), 2.28 (s, 2H), 1.02 (d, 3H, J=6.4 Hz), 0.91 (s, 3H), 0.70 (s, 3H).

Cholyl-β-Ala-Gabapentin (7n): MS (ESI): m/z 631.45 (M−H⁻), 633.45 (M+H⁺).

¹H NMR (CD₃OD, 400 MHz, characteristic resonances only): 3.34 (s, 2H), 3.20 (t, 2H, J=8 Hz), 2.29 (s, 2H), 2.26 (t, 2H, J=8 Hz), 1.02 (d, 3H, J=6.4 Hz), 0.91 (s, 3H), 0.70 (s, 3H).

Cholyl-Gaba-Gabapentin (7o): MS (ESI): m/z 645.56 (M−H⁻), 647.57 (M+H⁺).

¹H NMR (CD₃OD, 400 MHz, characteristic resonances only): 3.34 (s, 2H), 3.41 (t, 2H, J=6.8 Hz), 2.44 (t, 2H, J=6.8 Hz), 2.28 (s, 2H), 1.01 (d, 3H, J=6.4 Hz), 0.91 (s, 3H), 0.71 (s, 3H).

### Example 3

### Synthesis of Compound (8)

To a solution of cholic acid (1) (2.04 g, 5 mmol) in dry THF (100 mL) was added triethylamine (765 μL, 5.5 mmol) followed by 2,4,6-trichlorobenzoyl chloride (858 μL, 5.5 mmol). After 10 min a solution of 3-hydroxypropylnitrile (341 μL, 5 mmol) in dry THF was added followed by DMAP (65 mg). The mixture was stirred at room temperature for 18 h. The reaction mixture was washed with saturated NaHCO₃ (10 mL) then saturated aqueous citric acid (3×10 mL). The organic phase was dried over MgSO₄, the solvent removed in vacuo and the residue purified by flash chromatography on silica gel (CH₂Cl₂-MeOH 97:3) to give pure cyanoethyl cholate (8) (2.05 g, 89% yield).

MS (ESI): m/z=462.6 (M+H⁺).

¹H NMR (CD₃OD, 400 MHz, characteristic resonances only): 4.27 (t, 2H, J=6 Hz), 2.70 (t, 2H, J=6 Hz), 0.99 (d, 3H, J=6.4 Hz), 0.88 (s, 3H), 0.68 (s, 3H).

### Example 4

### Synthesis of Compound (II)

A solution containing ethyl 6-hydroxyhexanoate (9) (162 μL, 1 mmol), 3,4-dihydro-2H-pyran (137 μL, 1.5 mmol) and pyridium p-toluenesulfonate (25 mg, 0.1 mmol) in dry CH₂Cl₂ (10 mL) was stirred at room temperature for 4 h. CH₂Cl₂ (10 mL) was added and the reaction mixture and washed with brine (3×5 mL). The organic phase was dried over MgSO₄ and evaporated to dryness yielding (10). The resulting residue was treated with aqueous 0.5 N NaOH (10 mL) and MeOH (10 mL) at 60° C. for 2 h. After removal of MeOH in vacuo and washing with CH₇Cl₂ (10 mL), the aqueous phase was acidified with citric acid. Extraction with ether (3×15 mL) and concentration in vacuo gave the THP-protected hydroxy-acid (11) (216 mg, 100% yield), which was used without further purification.

MS (ESI): m/z=215.3 (M−H⁻).

### Example 5

### Synthesis of Compound (17)

1,1-Cyclohexanediacetic acid (4 g, 20 mmol) and acetic anhydride (3.8 mL, 40 mmol) were heated under reflux until a clear solution was obtained (˜1 h), and heating continued for a further hour to ensure the reaction had gone to completion. The mixture was cooled to room temperature and the solvent removed in vacuo to afford 1,1-cyclohexanediacetic anhydride (15) (3.6 g, 99% yield).

MS (ESI): m/z=183.2 (M+H⁺).

(15) (3.6 g 19.7 mmol) was stirred in 0.5M sodium methoxide/MeOH solution (40 mL) at room temperature for 2 h. After removal of the solvent in vacuo, 0.5 N HCl (20 mL) was added to the residue and the product extracted with EtOAc (3×30 mL). The combined organic phase was dried over MgSO₄ and concentrated in vacuo to give monomethyl ester (16) (4 g, 95% yield).

MS (ESI): m/z 213.3 (M−H⁻).

To a solution of (16) (1.6 g, 7.5 mmol) in anhydrous acetone (10 mL) was slowly added triethylamine (1.25 mL, 9 mmol). The solution was cooled to −5 to 0° C. in an ice-salt bath and ethyl chloroformate (0.89 mL, 9 mmol) in anhydrous acetone (10 mL) was added dropwise, maintaining the temperature between −5 to 0° C. After addition was complete, the cold mixture was stirred for an additional 15 min. A solution of sodium azide (975 mg, 15 mmol) in water (3 mL) was then added slowly, the temperature being maintained between −5 to 0° C. The mixture was stirred for an additional 30 min, poured into ice water (5 mL), and shaken with toluene (4×25 mL). The combined toluene extracts were dried over MgSO₄ and the resulting acyl azide (17) used immediately in a Curtius reaction with the appropriate alcohol (vide infra).

### Example 6

### Synthesis of Compound (18)

(8) (120 mg, 0.26 mmol) was heated under reflux with a toluene solution containing acyl azide (17) (˜2.5 mmol) for 14 h. After cooling to room temperature, the solvent was removed in vacuo and the residue dissolved in EtOAc (20 mL), washed with water (2×10 mL) and dried over MgSO₄. The cyanoethyl ester product (40 mg, 23% yield) was purified using preparative TLC (10% MeOH/CH₂Cl₂).

MS (ESI): m/z=673.5 (M+H⁺).

This material was treated with 20% piperidine/CH₂Cl₂ (2 mL) for 30 min and the solvent removed in vacuo. Purification of the resulting residue by preparative TLC (10% MeOH/CH₂Cl₂) afforded the gabapentin carbamate conjugate (18) (28 mg, 77% yield).

MS (ESI): m/z=620.6 (M+H⁺).

¹H NMR (CD₃OD, 400 MHz, characteristic resonances only): 3.88 (s, 2H), 3.65 (s, 3H), 3.34 (s, 2H), 2.28 (s, 2H), 1.02 (d, 3H, J=6.4 Hz), 0.91 (s, 3H), 0.71 (s, 3H).

### Example 7

### Synthesis of Compound (19)

To a solution of (11) (216 mg, 1 mmol) in dry CH₂Cl₂ (10 mL) was added triethylamine (167 μL, 1.2 mmol) followed by 2,4,6-trichlorobenzoylchloride (187 μL, 1.2 mmol). After 10 min, a solution of (12) (521 mg, 1 mmol) in dry CH₂Cl₂ (20 mL) was added dropwise, followed by DMAP (12 mg). The reaction mixture was stirred at room temperature for 18 h, then washed with saturated aqueous NaHCO₃ (10 mL) and saturated aqueous citric acid (3×10 mL). The organic phase was dried over MgSO₄ and purified by flash chromatography on silica gel (CH₂Cl₂-MeOH 97:3) to give compound (13) (345 mg, 48% yield).

MS (ESI): m/z=742.6 (M+Na⁺).

¹H NMR (CD₃OD, 400 MHz, characteristic resonances only): 3.91 (s, 2H), 1.44 (s, 9H), 0.97 (d, 3H, J=6.4 Hz), 0.88 (s, 3H), 0.67 (s, 3H).

A mixture of (13) (230 mg, 0.32 mmol) and pyridium p-toluenesulfonate (8 mg, 0.032 mmol) in MeOH (10 mL) was stirred at 55° C. for 4 h. The solvent was removed in vacuo, and the residue purified by chromatography on silica gel to afford the pure alcohol intermediate (173 mg, 85% yield). Electrospray mass spectrometry showed the expected molecular ion at m/z=636.6 (M+H⁺). A sample of this product (48 mg, 0.075 mmol) was heated under reflux with a toluene solution containing acyl azide (17) (˜2.5 mmol) for 14 h. After cooling to room temperature, the solvent was removed in vacuo and the residue dissolved in EtOAc (20 mL), washed with water (2×10 mL) and dried over MgSO₄. This tert-butyl ester product (30 mg, 47% yield) was purified using preparative TLC (10% MeOH/CH₂Cl₂).

MS (ESI): m/z=847.63 (M+H⁺).

The ester was treated with 50% TFA/CH₂Cl₂ for 3 h, the solvent removed in vacuo and the resulting residue stirred for 30 min with 20% piperidine in CH₂Cl₂ (10 mL). After removal of the solvent in vacuo, the residue was purified using preparative TLC (10% MeOH/CH₂Cl₂) to afford glycocholate derivative (19) (15 mg, 54% yield).

MS (ESI): 791.6 (M+H⁺).

¹H NMR (CD₃OD, 400 MHz, characteristic resonances only): 3.88 (s, 2H), 3.65 (s, 3H), 3.34 (s, 2H), 2.28 (s, 2H), 1.02 (d, 3H, J=6.4 Hz), 0.91 (s, 3H), 0.71 (s, 3H).

### Example 8

### Synthesis of Cholyl-Dopa (20)

To an ice-cold solution containing cholic acid (1) (816 mg, 2 mmol) and triethylamine (0.556 mL, 4 mmol) in anhydrous THF (100 mL) was added ethyl chloroformate (0.211 mL, 2.2 mmol). The reaction mixture was stirred at 0° C. for 30 min. A solution of L-Dopa (788 mg, 4 mmol) and NaHCO₃ (420 mg, 5 mmol) in water (10 mL) was added at 0° C., then stirred for 30 min. at 0° C., and for a further 30 min. at room temperature. After removal of THF in vacuo, aqueous citric acid (20 mL) was added. The product was extracted with ethyl acetate (3×30 mL) and the combined organic phase was dried over MgSO₄ and concentrated to dryness. Chromatography on a silica gel column eluting with 5% MeOH/EtOAc gave the desired Cholyl-Dopa product (20) (880 mg, 75%).

MS (ESI) m/z 588.33 (M+H⁺).

¹H NMR (CD₃OD, 400 MHz, characteristic resonances only): 6.64 (d, 1H, J=8 Hz), 6.64 (d, 1H, J=2 Hz), 6.52 (dd, 1H, J=2 Hz, J=8 Hz), 4.56 (m, 1H), 3.06-2.75 (m, 2H), 0.98 (d, 3H, J=6.4 Hz), 0.91 (s, 3H), 0.68 (s, 3H).

### Example 9

### Synthesis of Cholyl-Dopa-(3,4-carbonate) (23)

(20) (59 mg, 0.1 mmol) was dissolved in anhydrous THF (30 mL), 1,1′-carbonyldiimidazole (32 mg, 0.2 mmol) was added and the mixture heated under reflux for 24 h. The reaction was monitored to completion by TLC (10% MeOH/EtOAc). After removal of the solvent in vacuo, the residue was dissolved in EtOAc, and washed with aqueous citric acid. The organic phase was dried over MgSO₄ and concentrated to dryness. Chromatography on a silica gel column eluting with 5% MeOH/EtOAc gave the desired cyclic carbonate product (23) (15 mg, 24%).

MS (ESI) m/z 614.38 (M+H⁺).

¹H NMR (CD₃OD, 400 MHz, characteristic resonances only): 7.25 (m, 2H), 7.17 (m, 1H), 4.09 (m, 1H), 2.92-2.77 (m, 2H), 0.98 (d, 3H, J=6.4 Hz), 0.90 (s, 3H), 0.69 (s, 3H).

### Example 10

### Synthesis of Cholyl-Dopa-(4-pivaloyloxymethyl) (21)

(20) (400 mg, 0.68 mmol) was dissolved in anhydrous acetone (20 mL), sodium carbonate (144 mg, 1.4 mmol) was added and the mixture stirred at room temperature for 15 min. In a separate flask, sodium iodide (300 mg, 2 mmol) was dissolved in anhydrous acetone (10 mL) and chloromethylpivalate (144 μL, 1 mmol) was added at once. After stirring at room temperature for 30 min, the in situ-generated iodomethylpivalate was transferred to the flask containing Cholyl-Dopa and sodium carbonate. The mixture was heated in an oil bath at 70° C. for 18 h. The reaction was monitored to completion by TLC (10% MeOH/EtOAc). After removal of the solvent in vacuo, the residue was dissolved in EtOAc and washed with aqueous citric acid and 0.1% Na₂S₂O₃. The organic phase was dried over MgSO₄ and concentrated to dryness. Chromatography on a silica gel column eluting with 2% MeOH/EtOAc gave the desired product Cholyl-Dopa-(4-pivaloyloxymethyl) (21) (210 mg, 44%).

MS (ESI) m/z 702.44 (M+H⁺).

¹H NMR (CD₃OD, 400 MHz, characteristic resonances only): 6.66 (d, 1H, J=8 Hz), 6.64 (d, 1H, J=2 Hz), 6.52 (dd, 1H, J=2 Hz, J=8 Hz), 5.55 (dd, 2H, J=2.8 Hz, J=17 Hz), 4.58 (m, 1H), 3.01-2.75 (m, 2H), 1.19 (s, 9H), 0.98 (d, 3H, J=6.4 Hz), 0.91 (s, 3H), 0.68 (s, 3H).

### Example 11

### Synthesis of Cholyl-Dopa-(4-acetoxymethyl) (22)

(20) (587 mg, 1 mmol) was dissolved in anhydrous acetone (30 mL), sodium carbonate (144 mg, 1.4 mmol) was added and the mixture stirred at room temperature for 15 min. Bromomethylacetate (155 μL, 1.5 mmol) was added and the mixture heated in an oil bath at 70° C. for 18 h. The reaction was monitored to completion by TLC (10% MeOH/EtOAc). After removal of the solvent in vacuo, the residue was dissolved in EtOAc and washed with aqueous citric acid. The organic phase was dried over MgSO₄ and concentrated to dryness. Chromatography on a silica gel column eluting with 2% MeOH/EtOAc gave the desired product Cholyl-Dopa-(4-acetoxymethyl) (22) (240 mg, 36%).

MS (ESI) m/z 660.22 (M+H⁺).

¹H NMR (CD₃OD, 400 MHz, characteristic resonances only): 6.66 (d, 1H, J=8 Hz), 6.63 (d, 1H, J=2 Hz), 6.51 (dd, 1H, J=2 Hz, J=8 Hz), 5.72 (dd, 2H, J=2.8 Hz, J=15.2 Hz), 4.56 (m, 1H), 3.02-2.75 (m, 2H), 2.06 (s, 3H), 0.98 (d, 3H, J=6.4 Hz), 0.90 (s, 3H), 0.68 (s, 3H).

### Example 12

### Synthesis of H-Glu-Cys(Bzl)-OH (27)

Into a 40 mL vial was added H-Cys(Bzl)-OH (25) (1.27 g, 6 mmol), water (10 mL), pyridine (1 mL), and DMA (10 mL). To the reaction was added Boc-Glu(OtBu)-OSu (24) (4.0 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-Cys(Bzl)-OH as a white solid.

MS (ESI) m/z 495.19 (M−H⁻).

Into a 20 mL vial was added a portion of Boc-Glu(OtBu)-Cys(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Glu-Cys(Bzl)-OH (27) as a white powder.

MS (ESI) m/z 339.18 (M−H⁻), 341.13 (M+H⁺).

### Example 13

### Synthesis of H-Glu-Ser(Bzl)-OH (28)

Into a 40 mL vial was added H-Ser(Bzl)-OH (26) (17 g, 6 mmol), water (10 mL), pyridine (1 mL), and DMA (10 mL). To the reaction was added Boc-Glu(OtBu)-OSu (24) (4.0 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-Ser(Bzl)-OH as a white solid.

MS (ESI) m/z 479.27 (M−H⁻).

Into a 20 mL vial was added a portion of Boc-Glu(OtBu)-Ser(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Glu-Ser(Bzl)-OH (28) as a white powder.

MS (ESI) m/z 323.28 (M−H⁻), 325.19 (M+H⁺).

### Example 14

### Synthesis of H-Asp-Cys(Bzl)-OH (30)

Into a 40 mL vial was added H-Cys(Bzl)-OH (25) (1.27 g, 6 mmol), water (10 mL), pyridine (1 mL), and DMA (10 mL). To the reaction was added Boc-Asp(OtBu)-OSu (29) (3.86 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, mL) and filtered through a 0.2 μm nylon membrane filter. The solution was purified by preparative HPLC. The pure fractions were combined and concentrated under reduced pressure to afford the product Boc-Asp(OtBu)-Cys(Bzl)-OH as a white solid.

MS (ESI) m/z 481.14 (M−H⁻).

Into a 20 mL vial was added a portion of Boc-Asp(OtBu)-Cys(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Asp-Cys (Bzl)-OH (30) as a white powder.

MS (ESI) m/z 325.24 (M−H⁻), 327.13 (M+H⁺).

### Example 15

### Synthesis of H-Glu-D-Cys(Bzl)-OH (34)

Into a 40 mL vial was added H-D-Cys(Bzl)-OH (32) (1.27 g, 6 mmol), water (10 mL), pyridine (1 mL), and DMA (10 mL). To the reaction was added Boc-Glu(OtBu)-OSu (24) (4.0 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-D-Cys(Bzl)-OH as a white solid.

MS (ESI) m/z 495.19 (M−H⁻).

Into a 20 mL vial was added a portion of Boc-Glu(OtBu)-D-Cys(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Glu-D-Cys(Bzl)-OH (34) as a white powder.

MS (ESI) m/z 339.19 (M−H⁻), 341.11 (M+H⁺).

### Example 16

### Synthesis of H-Glu-D-Ser(Bzl)-OH (35)

Into a 40 mL vial was added H-D-Ser(Bzl)-OH (33) (1.17 g, 6 mmol), water (10 mL), pyridine (1 mL), and DMA (10 mL). To the reaction was added Boc-Glu(OtBu)-OSu (24) (4.0 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-D-Ser(Bzl)-OH as a white solid.

MS (ESI) m/z 479.20 (M−H⁻).

Into a 20 mL vial was added a portion of Boc-Glu(OtBu)-D-Ser(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Glu-D-Ser(Bzl)-OH (35) as a white powder.

MS (ESI) m/z 323.16 (M−H⁻), 325.16 (M+H⁺).

### Example 17

### Synthesis of H-Asp-D-Cys(Bzl)-OH (36)

Into a 40 mL vial was added H-D-Cys(Bzl)-OH (32) (1.27 g, 6 mmol), water (10 mL), pyridine (1 mL), and DMA (10 mL). To the reaction was added Boc-Asp(OtBu)-OSu (29) (3.86 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Asp(OtBu)-Cys(Bzl)-OH as a white solid.

MS (ESI) m/z 481.14 (M−H⁻).

Into a 20 mL vial was added a portion of Boc-Asp(OtBu)-D-Cys(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Asp-D-Cys-(Bzl)-OH (36) as a white powder.

MS (ESI) m/z 325.15 (M−H⁻), 327.14 (M+H⁺).

### Example 18

### Synthesis of H-Asp-D-Ser(Bzl)-OH (37)

Into a 40 mL vial was added H-D-Ser(Bzl)OH (33) (1.17 g, 6 mmol), water (10 mL), pyridine (1 mL), and DMA (10 mL). To the reaction was added Boc-Asp(OtBu)-OSu (29) (3.86 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Asp(OtBu)-D-Ser(Bzl)-OH as a white solid.

MS (ESI) m/z 465.20 (M−H⁻).

Into a 20 mL vial was added a portion of Boc-Asp(OtBu)-D-Ser(Bzl)-OH and TFA (10 mL). The reaction was kept at ambient temperature for 16 hours. The solvent was removed under reduced pressure. The residue was dissolved in water (4 mL) and filtered through a 0.2 μm nylon membrane filter. The solution was purified by preparative HPLC. The pure fractions were combined and concentrated under reduced pressure to afford the product H-Asp-D-Ser(Bzl)-OH (37) as a white powder

MS (ESI) m/z 309.15 (M−H⁻), 311.16 (M+H⁺).

### Example 19

### Synthesis of H-Glu-Cys(Cyclohexylcarbonyl)-OH (45)

Into a 40 mL vial was added H-Cys-OH (38) (1.45 g, 12 mmol), water (20 mL), pyridine (2 mL), and DMA (10 mL). To the reaction was added Boc-Glu(OtBu)-OSu (24) (4.0 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-Cys-OH (40) as a white solid.

MS (ESI) m/z 405.24 (M−H⁻).

Into a 40 mL vial was added Boc-Glu(OtBu)-Cys-OH (40) (1.2 g, 3 mmol), CH₂Cl₂ (20 mL), pyridine (0.5 mL), and cyclohexanecarbonyl chloride (0.4 mL, 3 mmol). The reaction mixture was shaken at ambient temperature for 2 days. The solvent was removed under reduced pressure. The residue was dissolved in MeCN (4 mL) and filtered through a 0.2 μm nylon membrane filter. The solution was purified by preparative HPLC. The pure fractions were combined and concentrated under reduced pressure to afford the product Boc-Glu(OtBu)-Cys(cyclohexylcarbonyl)-OH as a white solid.

MS (ESI) m/z 515.20 (M−H⁻), 517.15 (M+H⁺).

Into a 20 mL vial is added a portion of Boc-Glu(OtBu)-Cys(cyclohexylcarbonyl)-OH and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Glu-Cys(cyclohexylcarbonyl)-OH (45) as a white powder.

### Example 20

### Synthesis of H-Asp-Cys(Cyclohexylcarbonyl)-OH (46)

Into a 40 mL vial was added H-Cys-OH (38) (1.45 g, 12 mmol), water (20 mL), pyridine (2 mL), and DMA (10 mL). To the reaction was added Boc-Asp(OtBu)-OSu (29) (3.86 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Asp(OtBu)-Cys-OH (42) as a white solid.

MS (ESI) m/z 391.13 (M−H⁻).

Into a 40 mL vial was added Boc-Asp(OtBu)-Cys-OH (42) (1.2 g, 3 mmol), CH₂Cl₂ (20 mL), pyridine (0.5 mL), and cyclohexanecarbonyl chloride (0.4 mL, 3 mmol). The reaction mixture was shaken at ambient temperature for 2 days. The solvent was removed under reduced pressure. The residue was dissolved in MeCN (4 mL) and filtered through a 0.2 μm nylon membrane filter. The solution was purified by preparative HPLC. The pure fractions were combined and concentrated under reduced pressure to afford the product Boc-Asp(OtBu)-Cys(cyclohexylcarbonyl)-OH as a white solid.

MS (ESI) m/z 501.16 (M−H⁻), 503.06 (M+H⁺).

Into a 20 mL vial is added a portion of Boc-Asp(OtBu)-Cys(cyclohexylcarbonyl)-OH and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Asp-Cys(cyclohexylcarbonyl)-OH (46) as a white powder.

### Example 21

### Synthesis of H-Glu-Cys(4-Chlorobenzoyl)-OH (48)

Into a 40 mL vial was added Boc-Glu(OtBu)-Cys-OH (40) (1.2 g, 3 mmol), CH₂Cl₂ (20 mL), pyridine (0.5 mL), and 4-chlorobenzoyl chloride (0.38 mL, 3 mmol). The reaction mixture was shaken at ambient temperature for 2 days. The solvent was removed under reduced pressure. The residue was dissolved in MeCN (4 mL) and filtered through a 0.2 μm nylon membrane filter. The solution was purified by preparative HPLC. The pure fractions were combined and concentrated under reduced pressure to afford the product Boc-Glu(OtBu)-Cys(4-chlorobenzoyl)-OH as a white solid.

MS (ESI) m/z 543.16 (M−H⁻), 545.05 (M+H⁺).

Into a 20 mL vial is added a portion of Boc-Glu(OtBu)-Cys(4-chlorobenzoyl)-OH and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Glu-Cys(4-chlorobenzoyl)-OH (48) as a white powder.

### Example 22

### Synthesis of H-Asp-Cys(4-Chlorobenzoyl)-OH (49)

Into a 40 mL vial was added Boc-Asp(OtBu)-Cys-OH (42) (1.2 g, 3 mmol), CH₂Cl₂ (20 mL), pyridine (0.5 mL), and 4-chlorobenzoyl chloride (0.38 mL, 3 mmol). The reaction mixture was shaken at ambient temperature for 2 days. The solvent was removed under reduced pressure. The residue was dissolved in MeCN (4 mL) and filtered through a 0.2 μm nylon membrane filter. The solution was purified by preparative HPLC. The pure fractions were combined and concentrated under reduced pressure to afford the product Boc-Asp(OtBu)-Cys(4-chlorobenzoyl)-OH as a white solid.

MS (ESI) m/z 529.09 (M−H⁻), 531.06 (M+H⁺).

Into a 20 mL vial is added a portion of Boc-Asp(OtBu)-Cys(4-chlorobenzoyl)-OH and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Asp-Cys(4-chlorobenzoyl)-OH (49) as a white powder.

### Example 23

### Synthesis of H-Glu-Cys(N-Phthaloylglycyl)-OH (51)

Into a 40 mL vial is added Boc-Glu(OtBu)-Cys-OH (40) (1.2 g, 3 mmol), CH₂Cl₂ (20 mL), pyridine (0.5 mL), and N-phthaloylglycyl chloride (50) (0.67 g, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Glu(OtBu)-Cys(N-phthaloylglycyl)-OH as a white solid.

Into a 20 mL vial is added a portion of Boc-Glu(OtBu)-Cys(N-phthaloylglycyl)-OH and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Glu-Cys(N-phthaloylglycyl)-OH (51) as a white powder.

### Example 24

### Synthesis of H-Asp-Cys(4-Chlorobenzoyl)-OH (52)

Into a 40 ml, vial is added Boc-Asp(OtBu)-Cys-OH (42) (1.2 g, 3 mmol), CH₂Cl₂ (20 mL), pyridine (0.5 mL), and N-phthaloylglycyl chloride (50) (0.67 g, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Asp(OtBu)-Cys(N-phthaloylglycyl)-OH as a white solid.

Into a 20 mL vial is added a portion of Boc-Asp(OtBu)-Cys(N-phthaloylglycyl)-OH and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Asp-Cys(N-phthaloylglycyl)-OH (52) as a white powder.

### Example 25

### Synthesis of Boc-Glu(OtBu)-Cys-Gly-OtBu (55)

Into a 40 mL vial was added H-Cys(Trt)-OH (53) (2.18 g, 6 mmol), water (10 mL), pyridine (1 mL), and DMA (10 mL). To the reaction was added Boc-Glu(OtBu)-OSu (24) (4.0 g, 10 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-Cys(Trt)-OH as a white solid.

MS (ESI) m/z 647.28 (M−H⁻).

Into a 40 mL vial was added Boc-Glu(OtBu)-Cys(Trt)-OH (1.5 g, 2.3 mmol), dicyclohexylcarbodiimide (0.516 g, 2.5 mmol), N-hydroxysuccinamide (0.288 g, 2.5 mmol), and acetonitrile (20 mL). The reaction mixture was shaken at 22-25° C. for 4 hours. The precipitated dicyclohexylurea was removed by filtration. To the filtrate was added an aqueous solution (30 mL) of H-Gly-OtBu.HCl (54) (0.42 g, 2.5 mmol), and sodium hydroxide (0.12 g, 3 mmol). The reaction was stirred at 22-25 C for 48 hours. The reaction mixture was diluted with ethyl acetate/diethyl ether (1/1, 100 mL) and washed with aqueous NaHCO₃ (200 mL), 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Glu(OtBu)-Cys(Trt)-Gly-OtBu as a white solid.

MS (ESI) m/z 760.42 (M−H⁻).

Into a 40 mL vial is added a portion of Boc-Glu(OtBu)-Cys(Trt)-Gly-OtBu and 1% TFA in CH₂Cl₂ (20 mL). The reaction is kept at ambient temperature for 30 minutes. The solvent is removed under reduced pressure. The residue was dissolved in water (4 mL) and filtered through a 0.2 μm nylon membrane filter. The solution is purified by preparative HPLC. The pure fractions are combined and concentrated under reduced pressure to afford the product Boc-Glu(OtBu)-Cys-Gly-OtBu (55) as a white powder.

### Example 26

### Synthesis of Boc-Asp(OtBu)-Cys-Gly-OtBu (56)

Into a 40 mL vial was added H-Cys(Trt)-OH (53) (2.18 g, 6 mmol), water (10 mL), pyridine (1 mL), and DMA (10 mL). To the reaction was added Boc-Asp(OtBu)-OSu (29) (1.93 g, 5 mmol). The reaction mixture was shaken at ambient temperature for 3 days. The reaction mixture was diluted with ethyl acetate/diethyl ether (1/1, 100 mL) and washed with 0.5 M aqueous citric acid (2×100 mL) and water (2×100 mL). The organic phase was separated, dried (Na₂SO₄), filtered and concentrated under reduced pressure. The residue was dissolved in MeCN/water (1/1, 10 mL) and filtered through a 0.2 μm nylon membrane filter. The solution was purified by preparative HPLC. The pure fractions were combined and concentrated under reduced pressure to afford the product Boc-Asp(OtBu)-Cys(Trt)-OH as a white solid.

MS (ESI) m/z 633.33 (M−H⁻).

Into a 40 mL vial was added Boc-Asp(OtBu)-Cys(Trt)-OH (0.8 g, 1.3 mmol), dicyclohexylcarbodiimide (0.29 g, 1.4 mmol), N-hydroxysuccinamide (0.16 g, 1.4 mmol), and acetonitrile (20 mL). The reaction mixture was shaken at 22-25° C. for 4 hours. The precipitated dicyclohexylurea was removed by filtration. To the filtrate was added an aqueous solution (30 mL) of H-Gly-OtBu.HCl (54) (0.25 g, 1.5 mmol), and sodium hydroxide (0.12 g, 3 mmol). The reaction was stirred at 22-25 C for 48 hours. The reaction mixture was diluted with ethyl acetate/diethyl ether (1/1, 100 mL) and washed with aqueous NaHCO₃ (200 mL), 0.5 M aqueous citric acid (2×100 mL) and water (2×100 mL). The organic phase was separated, dried (Na₂SO₄), filtered and concentrated under reduced pressure to afford the product Boc-Asp(OtBu)-Cys(Trt)-Gly-OtBu as a white solid.

Into a 40 mL vial is added a portion of Boc-Asp(OtBu)-Cys(Trt)-Gly-OtBu and 1% TFA in CH₂Cl₂ (20 mL). The reaction is kept at ambient temperature for 30 minutes. The solvent is removed under reduced pressure. The residue was dissolved in water (4 mL) and filtered through a 0.2 μm nylon membrane filter. The solution is purified by preparative HPLC. The pure fractions are combined and concentrated under reduced pressure to afford the product Boc-Asp(OtBu)-Cys-Gly-OtBu (56) as a white powder.

### Example 27

### Synthesis of H-Glu-Cys(Cyclohexylcarbonyl)-Gly-OH (57)

Into a 40 mL vial is added Boc-Glu(OtBu)-Cys-Gly-OtBu (55) (3 mmol), CH₂Cl₂ (20 mL), pyridine (0.5 mL), and cyclohexanecarbonyl chloride (44) (0.4 mL, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Glu(OtBu)-Cys(cyclohexylcarbonyl)-Gly-OtBu as a white solid.

Into a 20 mL vial is added a portion of Boc-Glu(OtBu)-Cys(cyclohexylcarbonyl)-OtBu and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Glu-Cys(cyclohexylcarbonyl)-Gly-OH (57) as a white powder.

### Example 28

### Synthesis of H-Asp-Cvs(Cyclohexylcarbonyl)-Gly-OH (58)

Into a 40 mL vial is added Boc-Asp(OtBu)-Cys-Gly-OtBu (56) (3 mmol), CH₂Cl₂ (20 mL), pyridine (0.5 mL), and cyclohexanecarbonyl chloride (44) (0.4 mL, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Asp(OtBu)-Cys(cyclohexylcarbonyl)-Gly-OtBu as a white solid.

Into a 20 mL vial is added a portion of Boc-Asp(OtBu)-Cys(cyclohexylcarbonyl)-OtBu and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Asp-Cys(cyclohexylcarbonyl)-Gly-OH (58) as a white powder.

### Example 29

### Synthesis of H-Glu-Cys(4-Chlorobenzoyl)-Gly-OH (59)

Into a 40 mL vial is added Boc-Glu(OtBu)-Cys-Gly-OtBu (55) (31 mmol), CH₂Cl₂ (20 mL), pyridine (0.5 mL), and 4-chlorobenzoyl chloride (47) (0.38 mL, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Glu(OtBu)-Cys(4-chlorobenzoyl)-Gly-OtBu as a white solid.

Into a 20 mL vial is added a portion of Boc-Glu(OtBu)-Cys(4-chlorobenzoyl)-OtBu and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Glu-Cys(4-chlorobenzoyl)-Gly-OH (59) as a white powder.

### Example 30

### Synthesis of H-Asp-Cys(4-Chlorobenzoyl)-Gly-OH (60)

Into a 40 mL vial is added Boc-Asp(OtBu)-Cys-Gly-OtBu (56) (3 mmol), CH₂Cl₂ (20 mL), pyridine (0.5 mL), and 4-chlorobenzoyl chloride (47) (0.38 mL, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Asp(OtBu)-Cys(4-chlorobenzoyl)-Gly-OtBu as a white solid.

Into a 20 mL vial is added a portion of Boc-Asp(OtBu)-Cys(4-chlorobenzoyl)-OtBu and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Asp-Cys(4-chlorobenzoyl)-Gly-OH (60) as a white powder.

### Example 31

### Synthesis of H-Glu-Cvs(N-Phthaloylglycyl)-Gly-OH (61)

Into a 40 mL vial is added Boc-Glu(OtBu)-Cys-Gly-OtBu (55) (3 mmol), CH₂Cl₂ (20 mL), pyridine (0.5 mL), and N-phthaloylglycyl chloride (50) (0.67 g, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Glu(OtBu)-Cys(N-phthaloylglycyl)-Gly-OtBu as a white solid.

Into a 20 mL vial is added a portion of Boc-Glu(OtBu)-Cys(N-phthaloylglycyl)-OtBu and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Glu-Cys(N-phthaloylglycyl)-Gly-OH (61) as a white powder.

### Example 32

### Synthesis of H-Asp-Cys(N-Phthaloylglycyl)-Gly-OH (62)

Into a 40 mL vial is added Boc-Asp(OtBu)-Cys-Gly-OtBu (56) (3 mmol), CH₂Cl₂ (20 mL), pyridine (0.5 mL), and N-phthaloylglycyl chloride (50) (0.67 g, 3 mmol). The reaction mixture is shaken at ambient temperature for 2 days. The solvent is removed under reduced pressure. The residue is dissolved in MeCN (4 mL) and filtered through a 0.2 μm nylon membrane filter. The solution is purified by preparative HPLC. The pure fractions are combined and concentrated under reduced pressure to afford the product Boc-Asp(OtBu)-Cys(N-phthaloylglycyl)-Gly-OtBu as a white solid.

Into a 20 mL vial is added a portion of Boc-Asp(OtBu)-Cys(N-phthaloylglycyl)-OtBu and 1/1 CH₂Cl₂/TFA (10 mL). The reaction is kept at ambient temperature for 16 hours. The solvent is removed under reduced pressure. The residue is dissolved in water (4 mL) and filtered through a 0.2 μm nylon membrane filter. The solution is purified by preparative HPLC. The pure fractions are combined and concentrated under reduced pressure to afford the product H-Asp-Cys(N-phthaloylglycyl)-Gly-OH (62) as a white powder

### Example 33

### In Vitro Compound Transport Assays with IBAT and LBAT-Expressing Cell Lines

(a) Inhibition of Radiolabeled Taurocholate Uptake

CHO cells transfected with either the IBAT or LBAT transporter were seeded into 96-well microtiter plates at 100,000 cells/well in 100 μL DMEM containing 10% serum, glutamine and Penstrep. After overnight incubation the media was removed and test compound (25 μL) added at 2× the final desired concentration. Tritiated taurocholate (50,000 CPM/well) was diluted with cold substrate to a final concentration of 5 μM and 25 μL/well of this mixture was added to the plate. After incubating for 1 h at room temperature the solution was removed and the plate washed 4× with PBS at 4° C. 200 μL/well of scintillant is added and the plate then read in a Wallac microbeta counter. The inhibition data is processed by standard methods to calculate an inhibition constant Kᵢ for the test compound.

(b) Analysis of Electrogenic Transport in Xenopus Oocytes

RNA preparation: Human IBAT and LBAT Transporter cDNAs were subcloned into a modified pGEM plasmid that contains 5′ and 3′ untranslated sequences from the Xenopus β-actin gene. These sequences increase RNA stability and protein expression. Plasmid cDNA was linearized and used as template for in vitro transcription (Epicentre Technologies transcription kit, 4:1 methylated:non-methylated GTP).

Xenopus oocyte isolation. Xenopus laevis frogs were anesthetized by immersion in Tricaine (1.5 g/mL in deionized water) for 15 min. Oocytes were removed and digested in frog ringer solution (90 mM NaCl, 2 mM KCl, 1 mM MgCl₂, 10 mM NaHEPES, pH 7.45, no CaCl₂) with 1 mg/mL collagenase (Worthington Type 3) for 80-100 min with shaking. The oocytes were washed 6 times, and the buffer changed to frog ringer solution containing CaCl₂ (1.8 mM). Remaining follicle cells were removed if necessary. Cells were incubated at 16° C., and each oocyte injected with 10-20 μg RNA in 45 μL solution.

Electrophysiology measurements. Transport currents were measured 2-14 days after injection, using a standard two-electrode electrophysiology set-up (Geneclamp 500 amplifier, Digidata 1320/PCLAMP software and ADInstruments hardware and software were used for signal acquisition). Electrodes (2-4 mΩ) were microfabricated using a Sutter Instrument puller and filled with 3M KCl. The bath was directly grounded (transporter currents were less than 0.3 μA). Bath flow was controlled by an automated perfusion system (ALA Scientific Instruments, solenoid valves).

For transporter pharmacology, oocytes were clamped at −60 to −90 mV, and continuous current measurements acquired using PowerLab Software and an ADInstruments digitizer. Current signals were lowpass filtered at 20 Hz and acquired at 4-8 Hz. All bath and drug-containing solutions were frog ringers solution containing CaCl₂. Drugs were applied for 10-30 seconds until the induced current reached a new steady-state level, followed by a control solution until baseline currents returned to levels that preceded drug application. The difference current (baseline subtracted from peak current during drug application) reflected the net movement of charge resulting from electrogenic transport and was directly proportional to transport rate. Recordings were made from a single oocyte for up to 60 min, enabling 30-40 separate compounds to be tested per oocyte. Compound-induced currents were saturable and gave half-maximal values at substrate concentrations comparable to radiolabel competition experiments. To compare results between oocytes expressing different levels of transport activity, a saturating concentration of glycodeoxycholate (100 μM) was used as a common reference to normalize results from test compounds. Using this normalization procedure Vₘₐₓ (i.e. maximal induced current) for different compounds tested on different oocytes could be compared.

TABLE 1
In vitro transport data for selected compounds
on IBAT-expressing cells

| COMPOUND | IC₅₀ (μM) | EC₅₀ (μM) | % Max. (GDC) |
|---|---|---|---|
| (4) | 36 | 70 | 67 |
| (7a) | 66 | 22 | 67 |
| (7g) | 92 | 140 | 28 |
| (19) | 7 | 58 | 28 |
| (18) | >100 | >100 | 0 |
| (20) | 83 | NT | 0 |
| (23) | 74 | NT | 25 |
| (21) | 91 | NT | 104 |

IC₅₀ data from radiolabeled competition assay in transporter-expressing CHO cells
EC₅₀ and % Max data (relative to glycodeoxycholate) from transporter-expressing oocytes
NT = not tested

TABLE 2
In vitro transport data for selected compounds
on LBAT-expressing cells

| COMPOUND | IC₅₀ (μM) | EC₅₀ (μM) | % Max. (GDC) |
|---|---|---|---|
| (4) | 8 | 19 | 38 |
| (7a) | 64 | NT | NT |
| (7g) | 0.5 | NT | NT |
| (19) | 1.7 | NT | NT |
| (18) | 0.7 | 31 | 140 |
| (20) | 5 | NT | NT |
| (23) | 1.8 | NT | NT |
| (21) | 0.2 | NT | NT |

IC₅₀ data from radiolabeled competition assay in transporter-expressing CHO cells
EC₅₀ and % Max data (relative to glycodeoxycholate) from transporter-expressing oocytes
NT = not tested

### Example 34

### In Vitro Compound Transport Assays with PEPT1 and PEPT2-Expressing Cell Lines Analysis of Electrogenic Transport in Xenopus Oocytes

RNA preparation: Rat and human PEPT1 and PEPT2 transporter cDNAs were subcloned into a modified pGEM plasmid that contains 5′ and 3′ untranslated sequences from the Xenopus β-actin gene. These sequences increase RNA stability and protein expression. Plasmid cDNA was linearized and used as template for in vitro transcription (Epicentre Technologies transcription kit, 4:1 methylated:non-methylated GTP).

Xenopus oocyte isolation. Xenopus laevis frogs were anesthetized by immersion in Tricaine (1.5 g/mL in deionized water) for 15 min. Oocytes were removed and digested in frog ringer solution (90 mM NaCl, 2 mM KCl, 1 mM MgCl₂, 10 mM NaHEPES, pH 7.45, no CaCl₂) with 1 mg/mL collagenase (Worthington Type 3) for 80-100 min with shaking. The oocytes were washed 6 times, and the buffer changed to frog ringer solution containing CaCl₂ (1.8 mM). Remaining follicle cells were removed if necessary. Cells were incubated at 16° C., and each oocyte injected with 10-20 μg RNA in 45 μL solution.

Electrophysiology measurements. Transport currents were measured 2-14 days after injection, using a standard two-electrode electrophysiology set-up (Geneclamp 500 amplifier, Digidata 1320/PCLAMP software and ADInstruments hardware and software were used for signal acquisition). Electrodes (2-4 mΩ) were microfabricated using a Sutter Instrument puller and filled with 3M KCl. The bath was directly grounded (transporter currents were less than 0.3 μA). Bath flow was controlled by an automated perfusion system (ALA Scientific Instruments, solenoid valves).

For transporter pharmacology, oocytes were clamped at −60 to −90 mV, and continuous current measurements acquired using PowerLab Software and an ADInstruments digitizer. Current signals were lowpass filtered at 20 Hz and acquired at 4-8 Hz. All bath and drug-containing solutions were frog ringers solution containing CaCl₂. Drugs were applied for 10-30 seconds until the induced current reached a new steady-state level, followed by a control solution until baseline currents returned to levels that preceded drug application. The difference current (baseline subtracted from peak current during drug application) reflected the net movement of charge resulting from electrogenic transport and was directly proportional to transport rate. Recordings were made from a single oocyte for up to 60 min, enabling 30-40 separate compounds to be tested per oocyte. Compound-induced currents were saturable and gave half-maximal values at substrate concentrations comparable to radiolabel competition experiments. To compare results between oocytes expressing different levels of transport activity, a saturating concentration of glycyl-sarcosine (1 mM) was used as a common reference to normalize results from test compounds. Using this normalization procedure Vₘₐₓ (i.e. maximal induced current) for different compounds tested on different oocytes could be compared.

TABLE 3
In vitro transport data for selected compounds
on hPEPT1-expressing cells

| COMPOUND | % Max. (Gly-Sar) |
|---|---|
| (27) | 41 |
| (28) | 58 |
| (30) | 55 |
| (34) | 1 |
| (35) | 1 |
| (36) | 2 |
| (37) | 1 |

% Max response (relative to Gly-Sar) from transporter-expressing oocytes at a test compound concentration of 1 mM

TABLE 4
In vitro transport data for selected compounds
on hPEPT2-expressing cells

| COMPOUND | % Max. (Gly-Sar) |
|---|---|
| (27) | 56 |
| (28) | 82 |
| (30) | 78 |
| (34) | 0 |
| (35) | 0 |
| (36) | 0 |
| (37) | 0 |

% Max response (relative to Gly-Sar) from transporter-expressing oocytes at a test compound concentration of 1 mM

### Example 35

### In Vitro Uptake of (4) by CHO Cells Transfected with IBAT or LBAT Evaluated by LC-MS/MS

Active transport of (4) by the bile acid transport system was evaluated in vitro by incubation of (4) or glycocholate (control substrate) with untransfected CHO K1 cells or CHO cells transfected with either IBAT or LBAT. Cells (10⁵ cells/mL) were incubated in 96 well plates with varying concentrations (0.06 to 1000 μM) of (4) or glycocholate for 10 min. Cells were then washed with Hank's Balanced Salt Solution (HBSS) and lysed and extracted by addition of 100 μL of water followed by sonication. Concentrations of (4) or glycocholate in cell extracts were determined by direct injection onto an API 2000 LC/MS/MS equipped with an Agilent 100 binary pump and autosampler. Separation was achieved using a Keystone BDS Hypersil 2×50 min column heated to 45° C. during the analysis. The mobile phases were: 0.1% formic acid in water (A) and 0.1% formic acid in acetonitrile (B). The gradient condition was: 5% B for 1 min, increasing to 90% B in 0.2 min, maintained for 2.8 min and returning to 5% B for 2 min. A TurboIonSpray source was used on the API 2000. The analysis was performed in the positive ion mode and MRM transitions of 466/412 and 562/154 were used in the analysis of glycocholate and (4), respectively. Ten microliters of the cell extracts were injected. Peaks were integrated using Analyst quantitation software. The method was linear for (4) or glycocholate over the concentration range 0.039 to 10 μM. Active uptake of (4) was observed for both bile acid transport systems indicating the potential for enterohepatic recirculation of the prodrug.

### Example 36

### In Vitro Enzymatic Release of Gabapentin from Cholyl-Gabapentin Conjugates

Sustained oral delivery of a drug molecule by attachment through a cleavable linker arm to an actively transported promoiety requires that the drug eventually be released from the drug/cleavable linker/transporter compound (prodrug) by enzymatic cleavage in one or more tissues of the enterohepatic circulation. The release of gabapentin (2) from the prodrugs (4), Cholyl-Phe-Gabapentin (7g) (and other Cholyl-Amino Acid-Gabapentin conjugates (7)) was evaluated in vitro using tissues representative of those involved in the enterohepatic circulation. For Cholyl-Amino Acid-Gabapentin conjugates these studies indicated that in vitro cleavage of the prodrug could occur via a stepwise process, with release of the gabapentin-containing dipeptide (e.g. Phe-Gabapentin) preceeding hydrolysis to liberate free gabapentin.

Tissues were obtained from commercial sources (e.g., Pel-Freez Biologicals, Rogers, Ariz., or GenTest Corporation, Woburn, Mass.). Stability of Cholyl-Phe-Gabapentin towards specific enzymes (e.g., carboxypeptidase A, cholylglycine hydrolase) was also evaluated by incubation with the purified enzyme. Experimental conditions used for the in vitro studies are described in Table 3 below. Each preparation was incubated with test compound at 37° C. for one hour. Aliquots (50 μL) were removed at 0, 30, and 60 min and quenched with 0.1% trifluoroacetic acid in acetonitrile. Samples were then centrifuged and analyzed by LC/MS/MS as described below.

The stability of gabapentin-containing dipeptides to purified aminopeptidase 1 and to Caco-2 homogenates was evaluated as follows:

Aminopeptidase Stability: Aminopeptidase 1 (Sigma catalog #A-9934) was diluted in deionised water to a concentration of 856 units/mL. Stability studies were conducted by incubating prodrug (5 μM) with 0.856 units/mL aminopeptidase 1 in 50 mM Tris-HCl buffer at pH 8.0 and 37° C. Concentrations of intact prodrug and released drug were determined at zero time and 60 minutes using LC/MS/MS.

Pancreatin Stability: Stability studies were conducted by incubating prodrug (5 μM) with 1% (w/v) pancreatin (Sigma, P-1625, from porcine pancreas) in 0.025 M Tris buffer containing 0.5 M NaCl (pH 7.5) at 37° C. for 60 min. The reaction was stopped by addition of 2 volumes of methanol. After centrifugation at 14,000 rpm for 10 min, the supernatant was removed and analyzed by LC/MS/MS.

Caco-2 Homogenate S9 Stability: Caco-2 cells were grown for 21 days prior to harvesting. Culture medium was removed and cell monolayers were rinsed and scraped off into ice-cold 10 mM sodium phosphate/0.15 M potassium chloride, pH 7.4. Cells were lysed by sonication at 4° C. using a probe sonicator. Lysed cells were then transferred into 1.5 mL centrifuge vials and centrifuged at 9000 g for 20 min at 4° C. The resulting supernatant (Caco-2 cell homogenate S9 fraction) was aliquoted into 0.5 mL vials and stored at −80° C. until used.

For stability studies, prodrug (5 μM) was incubated in Caco-2 homogenate S9 fraction (0.5 mg protein per mL) for 60 min at 37° C. Concentrations of intact prodrug and released drug were determined at zero time and 60 minutes using LC/MS/MS.

Concentrations of (2), (4), (7g) or Phe-Gabapentin in tissue extracts were determined by direct injection onto an API 2000 LCS/MS equipped with an Agilent 1100 binary pump and autosampler. Separation was achieved using a 3.5 μm Zorbax Ellipse XDB-C8 4.4×150 mm column heated to 45° C. during the analysis. The mobile phases were: 0.1% formic acid in water (A) and 0.1% formic acid in acetonitrile (B). The gradient condition was: 2% B for 0.5 min, increasing to 90% B in 2.0 min, maintained for 2.5 min and returning to 2% B for 2 min. A TurboIonSpray source was used on the API 2000. The analysis was performed in the positive ion mode and MRM transitions of 709.5/172.1 and 172.0/137.2 were used in the analysis of Cholyl-Phe-Gabapentin (7g) and gabapentin (2) respectively. Ten microliters of the sample extracts were injected. Peaks were integrated using Analyst quantitation software. The method was linear for (7g) or (2) over the concentration range 0.01 to 12.5 μg/mL and 0.002 to 2.5 μg/mL respectively.

For (4) these data indicate a slow rate of hydrolysis of the prodrug in plasma, liver, or intestine resulting in formation of gabapentin. Substantially faster release of gabapentin was catalyzed by cholylglycine hydrolase (the naturally occurring bacterial enzyme responsible for hydrolysis of glycocholate in vivo).

TABLE 5
In Vitro Enzymatic Release of Phe-Gabapentin
from Cholyl-Phe-Gabapentin (7g)

| | | | Percent of |
|---|---|---|---|
| | | | Phe- |
| | Substrate | | Gabapentin |
| Preparation | Concentration | Cofactors | Released in 60 min |
| Rat Plasma | 2.0 μM | None | NR |
| Human Plasma | 2.0 μM | None | NR |
| Rat Liver S9 | 2.0 μM | NADPH | NR |
| (0.5 mg/mL) | | | |
| Human Liver S9 | 2.0 μM | NADPH | NR |
| (0.5 mg/mL) | | | |
| Human Intestine | 2.0 μM | NADPH | NR |
| S9 | | | |
| (0.5 mg/mL) | | | |
| Cholylglycine | 0.8 μM | None | ~3 |
| Hydrolase | | | |
| (87 units/mL) | | | |
| Carboxypeptidase A | 2.0 μM | None | NR |
| (10 units/mL) | | | |

NR = Not released

TABLE 6
In Vitro Enzymatic Release of Gabapentin (2) from Phe-Gabapentin

| | | | Percent of |
|---|---|---|---|
| | Substrate | | Gabapentin |
| Preparation | Concentration | Cofactors | Released in 60 min |
| Rat Plasma | 2.0 μM | None | 19 |
| Human Plasma | 2.0 μM | None | NR |
| Rat Liver S9 | 2.0 μM | NADPH | 1 |
| (0.5 mg/mL) | | | |
| Human Liver S9 | 2.0 μM | NADPH | 1 |
| (0.5 mg/mL) | | | |
| Human Intestine | 2.0 μM | NADPH | 5 |
| S9 | | | |
| (0.5 mg/mL) | | | |
| Cholylglycine | 0.8 μM | None | NR |
| Hydrolase | | | |
| (87 units/mL) | | | |
| Carboxypeptidase A | 2.0 μM | None | NR |
| (10 units/mL) | | | |
| Caco-2 | 5.0 μM | None | 21 |
| Homogenate | | | |
| Aminopeptidase | 5.0 μM | None | 24 |

NR = Not released

TABLE 7
In Vitro Enzymatic Release of Gabapentin from (4)

| | | | Percent of |
|---|---|---|---|
| | Substrate | | Gabapentin |
| Preparation | Concentration | Cofactors | Released in 60 min |
| Rat Plasma | 2.0 μM | None | 0.55 |
| Human Plasma | 2.0 μM | None | 0.31 |
| Rat Liver S9 | 2.0 μM | NADPH | 1.67 |
| (0.5 mg/mL) | | | |
| Human Liver S9 | 2.0 μM | NADPH | 4.89 |
| (0.5 mg/mL) | | | |
| Human Intestine | 2.0 μM | NADPH | 1.31 |
| S9 | | | |
| (0.5 mg/mL) | | | |
| Cholylglycine | 0.8 μM | None | 35.31 |
| Hydrolase | | | |
| (87 units/mL) | | | |
| Carboxypeptidase A | 2.0 μM | None | NR |
| (10 units/mL) | | | |

NR = Not released

TABLE 8
In Vitro Enzymatic Release of Gabapentin (2) from Cholyl-Amino
Acid-Gabapentin Compounds (7) by Pancreatin

| COMPOUND | % (2) Released |
|---|---|
| Cholyl-Gly-Gabapentin (7a) | NR |
| Cholyl-Phe-Gabapentin (7g) | 4 |
| Cholyl-Tyr-Gabapentin (7h) | 40 |

NR = Not released

### Example 37

In Vitro Enzymatic Release of (20) and L-Dopa from (21)

The release of L-Dopa and the intermediate (20) from the prodrug (21) was evaluated in vitro using tissues representative of those involved in the enterohepatic circulation. Similarly, the release of L-Dopa from (20) was examined in the same tissue preparations. Tissues were obtained from commercial sources (e.g., Pel-Freez Biologicals, Rogers, Ariz., or GenTest Corporation, Woburn, Mass.). Stability of (21) towards specific enzymes (e.g., carboxypeptidase A, cholylglycine hydrolase) was also evaluated by incubation with the purified enzyme. Experimental conditions used for the in vitro studies are described in the following table. Each preparation was incubated with (21) at 37° C. for one hour. Aliquots (50 μL) were removed at 0, 30, and 60 ml and quenched with 0.1% trifluoroacetic acid in acetonitrile. Samples were then centrifuged and analyzed by LCMS/MS as described in Example 36 above.

TABLE 9
In Vitro Enzymatic Release of L-Dopa or (20) from (21)

| | | | Percent | Percent |
|---|---|---|---|---|
| | | | of L-Dopa | of (20) |
| | Substrate | | Released | Released |
| Preparation | Concentration | Cofactors | in 60 min | in 60 min* |
| Rat Plasma | 2.0 μM | None | NR | 75 |
| Human | 2.0 μM | None | NR | 90 |
| Plasma | | | | |
| Rat Liver S9 | 2.0 μM | NADPH | NR | 35 |
| (0.5 mg/mL) | | | | |
| Human Liver | 2.0 μM | NADPH | NR | 70 |
| S9 | | | | |
| (0.5 mg/mL) | | | | |
| Human | 2.0 μM | NADPH | NR | 95 |
| Intestine S9 | | | | |
| (0.5 mg/mL) | | | | |
| Cholylglycine | 0.8 μM | None | NR | NR |
| Hydrolase | | | | |
| (87 units/mL) | | | | |

NR—Not released
*(20) was further hydrolysed in vitro by cholylglycine hydrolase (95% in 60 min) to release L-Dopa.

### Example 38

### Sustained Release of Gabapentin from (4) Following Oral Administration to Rats

The pharmacokinetics of the prodrug (4) were examined in rats. Three groups of four male Sprague-Dawley rats (approx 200 g) with jugular cannulae each received one of the following treatments: A) a single bolus intravenous injection of gabapentin (25 mg/kg, as a solution in water); B) a single oral dose of gabapentin (25 mg/kg, as a solution in water) administered by oral gavage; C) a single oral dose of (4) (85.25 mg/kg, as a solution in water) administered by oral gavage. Animals were fasted overnight prior to dosing and until 4 hours post-dosing. Serial blood samples were obtained over 24 hours following dosing and blood was processed for plasma by centrifugation. Plasma samples were stored at −80° C. until analyzed. Concentrations of (4) or gabapentin in plasma samples were determined by LC/MS/MS as described in Example 35. Plasma (50 μL) was precipitated by addition of 100 mL of methanol and supernatent was injected directly onto the LC/MS/MS system. The method was linear for gabapentin over the concentration range 0.001 to 20 ng/mL and for (4) over the concentration range 0.01 to 10 ng/mL. Following oral administration of gabapentin, concentrations of gabapentin in plasma reached a maximum at 2.8±2.5 hours (Tₘₐₓ) and declined thereafter with a terminal half-life of 2.4±0.5 hours. The oral bioavailability of gabapentin was 87±18%. Following oral administration of (4), concentrations of intact (4) in plasma reached a maximum at ˜8 hours post-dosing and were sustained out to 24 hours (terminal half-life >12 hours). Concentrations of released gabapentin in plasma were similarly sustained out to 24 hours (half-life >12 hours). These data indicate that prodrug (4) is metabolized to gabapentin in vivo, and that substantially sustained release of gabapentin was achieved following oral administration of (4) compared to the relatively rapid clearance observed for oral gabapentin.

### Example 39

Secretion of (4) in Bile Following Oral Administration to Rats

Sustained release of gabapentin from a prodrug that is subject to enterohepatic recirculation requires that a proportion of the intact prodrug be absorbed after oral administration and subsequently secreted into the bile intact. The potential for enterohepatic recirculation of intact (4) was examined in rats with indwelling bile duct fistulae. A group of four male Sprague-Dawley rats (approx. 200 g) cannulated in both the jugular vein and the common bile duct each received a single oral dose of (4) (85.25 mg/kg, as a solution in water) by oral gavage. Serial blood samples were obtained over 24 hours following dosing and blood was processed for plasma by centrifugation. Bile was collected continuously in aliquots over 24 hours. Plasma and bile samples were frozen at −80° C. until analyzed. Concentrations of (4) or gabapentin in plasma samples were determined by LC/MS/MS as described in Example 38. Concentrations of intact (8) in bile were similarly determined by LC/MS/MS. Bile (20 μL) was diluted 1:1000 with methanol and injected directly onto the HPLC system. Concentrations of (4) in bile reached a maximum at ˜6 hours post-dosing and were sustained up to 24 hours. These data indicate that (4) was successfully transported across the intestine by the ileal bile acid transport system (IBAT) and further secreted into the bile by the liver bile acid transporter (LBAT). However, no gabapentin was detected in plasma of bile duct-cannulated rats, indicating that cleavage of the prodrug was dependent on enterohepatic recirculation.

### Example 40

### Sustained Release of Gabapentin (2) from Cholyl-Phe-Gabapentin (7g) Following Oral Administration to Rats

The pharmacokinetics of the prodrug Cholyl-Phe-Gabapentin (7g) were examined in rats. Three groups of four male Sprague-Dawley rats (approx 200 g) with jugular cannulae each received one of the following treatments: A) a single bolus intravenous injection of gabapentin (25 mg/kg, as a solution in water); B) a single oral dose of gabapentin (25 mg/kg, as a solution in water) administered by oral gavage; C) a single oral dose of (7g) (103.5 mg/kg, as a solution in water) administered by oral gavage. Animals were fasted overnight prior to dosing and until 4 hours post-dosing. Serial blood samples were obtained over 24 hours following dosing and blood was processed for plasma by centrifugation. Plasma samples were stored at −80° C. until analyzed. Concentrations of (7g) or (2) in plasma samples were determined by LC/MS/MS as described above. Plasma (50 μL) was precipitated by addition of 100 mL of methanol and supernatent was injected directly onto the LC/MS/MS system. Following oral administration of gabapentin, concentrations of gabapentin in plasma reached a maximum at 2.8±2.5 hours (Tₘₐₓ) and declined thereafter with a terminal half-life of 2.4±0.5 hours. The oral bioavailability of gabapentin was 87±18%. Following oral administration of Cholyl-Phe-Gabapentin (7g), concentrations of gabapentin in plasma reached a maximum at ˜7.1 hours post-dosing and declined thereafter with a terminal half-life of 5.1 hours. Concentrations of released gabapentin in plasma were sustained beyond 24 hours. These data indicate that prodrug Cholyl-Phe-Gabapentin (7g) is metabolized to gabapentin (2) in vivo, and that a substantially sustained release of gabapentin was achieved following oral administration of (7g) compared to the relatively rapid clearance observed for oral gabapentin.

### Example 41

### Oral Bioavailability of L-Dopa and (20) from the Prodrug (21)

The pharmacokinetics of the prodrug (21) were examined in rats. Three groups of four male Sprague-Dawley rats (200-300 g) with jugular cannulae each received one of the following treatments: A) a single bolus intravenous injection of L-Dopa (75 mg/kg, as a solution in water); B) a single oral dose of L-Dopa (75 mg/kg, as a solution in water) administered by gavage; C) a single oral dose of (21) (267 mg/kg, as a solution in PEG400) administered by gavage. Animals were fasted overnight prior to the study and until 4 hours post-dosing. Serial blood samples were obtained over 48 hours following dosing and blood was processed for plasma by centrifugation. Plasma samples were frozen at −80° C. until analyzed.

Concentrations of L-Dopa in plasma were determined by LC/MS/MS. Plasma (100 μL) was mixed with 10 μL of 500 μg/ml deuterated L-Dopa as internal std, 25 μl of 10% sodium metabisulfite, 300 μL of 2M tris containing 5% EDTA and 30 mg of acid washed aluminum oxide was added to extract L-Dopa. The alumina was washed four times with 300 μL water and extracted with 300 μL of 2.5% formic acid. The extract was analyzed using LC/MS/MS on a 3 μm Phenomenex Luna 4.6×150 mm column. The mobile phases were: A) 0.1% formic acid; B) Acetonitrile with 0.1% formic acid at a flow rate of 0.5 mL/min at 40° C. The gradient was 2% B increasing to 90% B over 3.5 min. The MRM transitions were 198.1/152.0 for L-Dopa and 202.0/155.0 for deuterated L-Dopa. The method was linear over the range 0.02 to 20 μg/mL and the limit of quantitation was 0.02 μg/mL.

Concentrations of (21), and intermediate (20), in plasma samples were determined by LC/MS/MS following precipitation of protein. Plasma (100 μL) was mixed with 300 μL of MeOH and centrifuged at 14,000 rpm for 10 min. The supernatant was analyzed by LC/MS/MS as described above. The MRM transitions were 702.6/152.1 for (21) and 588.5/534.3 for (20).

### Example 42

### Sustained Release of N-Phthaloylglycine from Prodrug (61) Following Oral Administration to Rats

The pharmacokinetics of the N-phthaloylglycine prodrug (61) is examined in rats. Three groups of four male Sprague-Dawley rats (approx 200 g) with jugular cannulae each received one of the following treatments: A) a single bolus intravenous injection of N-phthaloylglycine (100 mg/kg, as a solution in water); B) a single oral dose of N-phthaloylglycine (100 mg/kg, as a solution in water) administered by oral gavage; C) a single oral dose of (61) (240 mg/kg, as a solution in water) administered by oral gavage. Animals are fasted overnight prior to dosing and until 4 hours post-dosing. Serial blood samples are obtained over 24 hours following dosing and blood is processed for plasma by centrifugation. Plasma samples are stored at −80° C. until analyzed. Concentrations of N-phthaloylglycine or (61) in plasma samples are determined by LC/MS/MS. Plasma (50 μL) is precipitated by addition of 100 mL of methanol and supernatent is injected directly onto the LC/MS/MS system. The concentration of N-phthaloylglycine in plasma following oral administration to rats declines rapidly with a terminal half-life of ˜10 minutes. Following oral administration of prodrug (61), liberated N-phthaloylglycine is apparent within 30 min post dosing and plasma concentrations decline more slowly with a terminal half-life of >2 h. These data indicate that prodrug (61) is metabolized to N-phthaloylglycine in vivo, and that a substantially sustained release of N-phthaloylglycine is achieved following oral administration of (61) compared to the rapid clearance observed for oral N-phthaloylglycine.

1-13. (canceled)

14. A pharmaceutical composition, which upon oral administration to an animal exhibits an extended pharmacokinetic profile through interaction with an enterohepatic circulation of the animal, comprising a pharmaceutically acceptable carrier, excipient or diluent and a conjugate of formula (I-b): 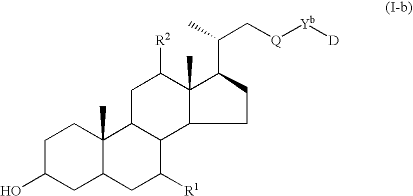
or pharmaceutically acceptable salts thereof, wherein:
Q is CH₂;
R¹ is selected from the group consisting of hydrogen and OH;
R² is selected from the group consisting of hydrogen and OH;
D is derived from a compound having therapeutic or prophylactic activity when delivered to the systemic circulation of the animal, wherein the compound is selected from the group consisting of a GABA analog, a L-dopa derivative, catechol O-methyl transferase inhibitor, a drug containing a carboxyl group, a drug containing an amine group, and a drug containing a hydroxyl group;
Y^{b} represents —C(O)—; and
wherein Y^{b} and D are selected so that Y cleaves from D and releases the compound having therapeutic or prophylactic activity at a rate that provides for therapeutic concentrations of the compound in said animal for a period of at least about 10% longer than that achieved by oral delivery of the compound itself from a similar oral dosage form.

15. The composition of claim 14, wherein D is derived from a GABA analog moiety having the structure: 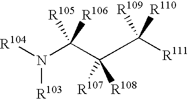
wherein
R¹⁰³ is selected from the group consisting of hydrogen, an amino-protecting group, or a covalent bond linking the moiety to Y^{b};
R¹⁰⁴ is hydrogen, or R¹⁰⁴ and R¹⁰⁹ together with the atoms to which they are attached form a heterocyclic ring;
R¹⁰⁵ and R¹⁰⁶ are independently selected from the group consisting of hydrogen, alkyl, substituted alkyl, alkenyl, substituted alkenyl, alkynyl, aryl, substituted aryl, heteroaryl and substituted heteroaryl;
R¹⁰⁷ and R¹⁰⁸ are independently selected from the group consisting of hydrogen, alkyl, substituted alkyl, alkenyl, alkynyl, aryl, substituted aryl, heteroaryl and substituted heteroaryl, or R¹⁰⁷ and R¹⁰⁸ together with the atoms to which they are attached form a cycloalkyl, substituted cycloalkyl, heterocyclic or substituted heterocyclic ring;
R¹⁰⁹ is selected from the group consisting of hydrogen, alkyl, substituted alkyl, alkenyl, alkynyl, aryl, substituted aryl, heteroaryl and substituted heteroaryl;
R¹¹⁰ is selected from the group consisting of hydrogen, alkyl, substituted alkyl, alkenyl, alkynyl, aryl, substituted aryl, heteroaryl and substituted heteroaryl;
R¹¹¹ is selected from the group consisting of carboxylic acid, carboxylic amide, carboxylic ester, sulfonamide, phosphonic acid, acidic heterocycle, sulfonic acid, hydroxamic acid and C(O)R¹¹²; and
R¹¹² is a covalent bond linking the GABA analog moiety to either Y^{b}, provided only one of R¹⁰³ and R¹¹² links the moiety to Y^{b};
with the proviso that D is not derived from gabapentin.

16. The composition of claim 14, wherein D is derived from an L-dopa derivative of formula (III): 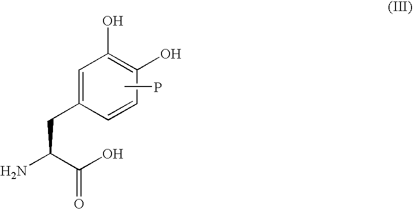
wherein:
P is selected from: 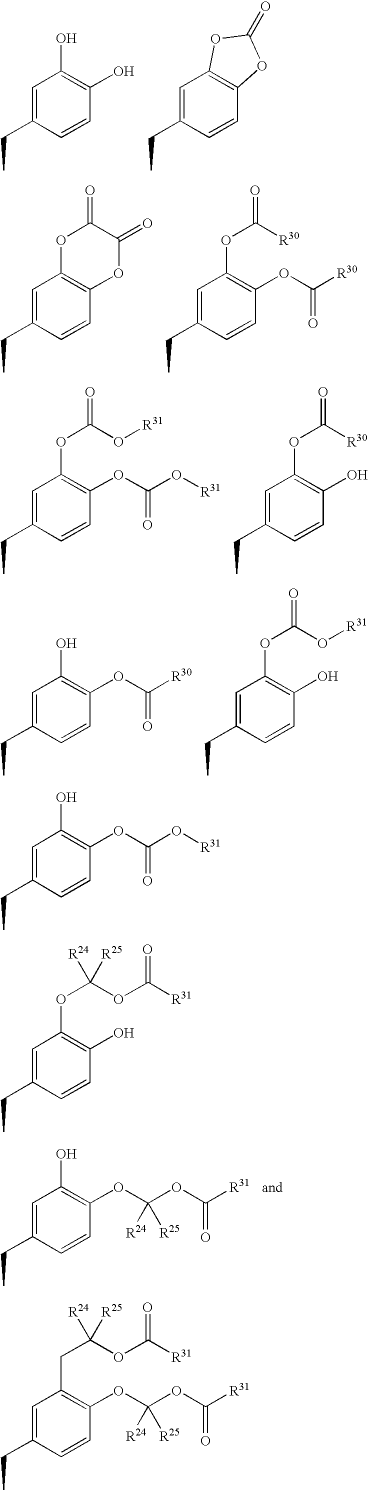
wherein:
R³⁰ is selected from hydrogen, alkyl, substituted alkyl, cycloalkyl, substituted cycloalkyl, aryl, substituted aryl, heteroaryl, and substituted heteroaryl;
R³¹ is selected from alkyl, substituted alkyl, cycloalkyl, substituted cycloalkyl, aryl, substituted aryl, heteroaryl, and substituted heteroaryl; and
each R²⁴ and R²⁵ are independently selected from hydrogen, alkyl, substituted alkyl, cycloalkyl, substituted cycloalkyl, aryl, substituted aryl, heteroaryl, and substituted heteroaryl; or R²⁴ and R²⁵ together with the carbon to which they are attached form a cycloalkyl, substituted cycloalkyl, heterocycloalkyl or substituted heterocycloalkyl ring;
with the proviso that one of the amino hydrogen atoms is removed to form a covalent bond to Y^{b}.

17. The composition of claim 14, wherein D is derived from a catechol O-methyl transferase inhibitor selected from entacapone, nitecapone, and tolcapone, optionally with one or two hydrogen atoms of two hydroxyl groups of the catechol group replaced with —C(O)R³⁰⁴, —C(O)OR³⁰⁵ and/or —OCR³⁰³R³⁰⁴OC(O)R³⁰⁵; wherein:
R³⁰³ and R³⁰⁴ independently are members selected from the group consisting of hydrogen, alkyl, substituted alkyl, cycloalkyl, substituted cycloalkyl, aryl, substituted aryl, aralkyl, substituted aralkyl, heteroaryl, and substituted heteroaryl; or R³⁰³ and R³⁰⁴ together with the carbon atom to which they are attached form a cycloalkyl, substituted cycloalkyl, heterocycle; or substituted heterocyclic ring; and
R³⁰⁵ is selected from the group consisting of alkyl, substituted alkyl, cycloalkyl, substituted cycloalkyl, aryl, substituted aryl, aralkyl, substituted aralkyl, heteroaryl, and substituted heteroaryl; or the OH group of the carboxyl moiety is replaced by —OR³⁰⁴;
with the proviso that one of the amino hydrogen atoms is removed to form a covalent bond to Y^{b}.

18. The composition of claim 14, wherein D is derived from a drug containing a carboxyl group selected from alecapril, captopril, 1-[4-carboxy-2-methyl-2R,4R-pentanoyl]-2,3-dihydro-2S-indole-2-carboxylic acid, enalaprilic acid, lisinopril, N-cyclopentyl-N-[3-[(2,2-dimethyl-1-oxopropyl)thio]-2-methyl-1-oxopropyl]glycine, pivopril, quinaprilat, (2R,4R)-2-hydroxyphenyl)-3-(3-mercaptopropionyl)-4-thiazolidinecarboxylic acid, (S)benzamido-4-oxo-6-phenylhexenoyl-2-carboxypyrrolidine, [2S-1[R*(R*))]]2α, 3αβ,7αβ]-1[2-[[1-carboxy-3-phenylpropyl]-amino]-1-oxopropyl]octahydro-1H-indole-2-carboxylic acid, [3S-1[R*(R*))]], 3R*]-2-[2-[[1-carboxy-3-phenylpropyl]-amino]-1-oxopropyl]-1,2,3,4-tetrahydro-3-isoquinolone carboxylic acid, tiopronin, cefaclor, cefadroxil, cefamandole, cefatrizine, cefazedone, cefazuflur, cefazolin, cefbuperazone, cefixime, cefmenoxime, cefmetazole, cefodizime, cefonicid, cefoperazone, ceforanide, cefotaxime, cefotefan, cefotiam, cefoxitin, cefpimizole, cefpirome, cefpodoxime, cefroxadine, cefsulodin, cefpiramide, ceftazidime, ceftezole, ceftizoxime, ceftriaxone, cefuroxime, cephacetrile, ceplialexin, cephaloglycin, cephaloridine, cephalosporin, cephanone, cephradine, latamoxef, amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperazillin, sulbenicllin, temocillin, ticarcillin, argatroban, melagatran, napsagatran, zanamivir, BCX-1812, acametacin, alclofenac, alminoprofen, aspirin, 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tolmetin, zopemirac, ciprostene, 16-deoxy-16-hydroxy-16-vinyl prostaglandin E₂, 6,16-dimethylprostaglandin E₂, epoprostostenol, meteneprost, nileprost, prostacyclin, prostaglandins E₁, E₂, or F_{2α} and thromboxane A₂, acrosoxacin, cinoxacin, ciprofloxacin, enoxacin, flumequine, naladixic acid, norfloxacin, ofloxacin, oxolinic acid, pefloxacin, pipemidic acid, piromidic acid, aztreonam, imipenem, and meropenem.

19. The composition of claim 14, wherein D is derived from a drug containing an amine group selected from acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, a minorex, aprinocid, cambendazole, cimetidine, cisapride, clonidine, cyclobenzadole, delavirdine, efegatrin, etintidine, fenbendazole, fenmetazole, flubendazole, fludorex, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, procainamide, ranitidine, tetrahydrazoline, tiamenidine, tinazoline, tiotidine, tocamide, tolazoline, tramazoline, xylometazoline, dimethoxyphenethylamine, N-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, clorprenaline, chlortermine, dopamine, ephrinephrine etryptamine, fenfluramine, methyldopamine, norepinephrine, tocamide, enviroxime, nifedipine, nimodipine, triamterene, norfloxacin, pipedemic acid, 1-ethyl-6-fluoro-1,4-dihydro-4-oxo-7-(1 piperazinyl)-1,8-napthyridine-3-carboxylic acid, and 1-cyclopropyl-6-fluoro-1,4-dihydro-4-oxo-7-(piperazinyl)-3-quinolinecarboxylic acid.

20. The composition of claim 14, wherein D is derived from a drug containing a hydroxy group selected from allylestrenol, cingestol, dehydroepiandrosteron, dienostrol, diethylstilbestrol, dimethisteron, ethyneron, ethynodiol, estradiol, estron, ethinyl estradiol, ethisteron, lynestrenol, mestranol, methyl testosterone, norethindron, norgestrel, norvinsteron, oxogeston, quinestrol, testosterone, tigestol, dofexazepam, hydroxyzin, lorazepam, oxazepam, acetophenazine, carphenazine, fluphenazine, perphenyzine, piperaetazine, aclarubicin, cytarabine, decitabine, daunorubicin, dihydro-5-azacytidine, doxonibicin, epirubicin, estramustin, etoposide, fludarabine, gemcitabine, 7-hydroxychlorpromazin, nelarabine, neplanocin A, pentostatin, podophyllotoxin, tezacitabine, troxacitabine, vinblastin, vincristin, vindesin, buserilin, gonadoliberin, icatibrant, leuprorelin acetate, terphenadine, diflunisal, naproxol, paracetamol, salicylamide, salicyclic acid, azidamphenicol, azithromycin, camptothecin, cefamandol, chloramphenicol, clarithromycin, clavulanic acid, clindamycin, demeclocyclin, doxycyclin, erythromycin, gentamycin, imipenem, latamoxef, metronidazole, neomycin, novobiocin, oleandomycin, oxytetracyclin, tetracycline, thiamenicol, tobramycin; antivirals, acyclovir, d4C, ddC, DMDC, Fd4C, FddC, FMAU, FTC, 2′-fluoro-ara-dideoxyinosine, ganciclovir, lamivudine, penciclovir, SddC, stavudine, 5-trifluoromethyl-2′-deoxyuridine, zalcitabine, zidovudine, EB-1053, etidronate, ibandronate, olpadronate, residronate, YH-529, zolendronate, ciprokiren, enalkiren, ritonavir, saquinavir, terlakiren, arbaprostil, carboprost, misoprostil, prostacydin, 8-hydroxychlorimipramine, 2-hydroxyimipramine, sotarol, fenoldopam, piperidine, procyclidin, trihexyphenidal, cromolyn, glucocorticoids, betamethasone, budenosid, chlorprednison, clobetasol, clobetasone, corticosteron, cortisone, cortodexon, dexamethason, flucortolon, fludrocortisone, flumethasone, flunisolid, fluprednisolon, flurandrenolide, flurandrenolon acetonide, hydrocortisone, meprednisone, methylpresnisolon, paramethasone, prednisolon, prednisol, triamcinolon, triamcinolon acetonide, apomorphine, buprenorphine, butorphanol, codein, cyclazocin, hydromorphon, ketobemidon, levallorphan, levorphanol, metazocin, morphine, nalbuphin, nalinefen, naloxon, nalorphine, naltrexon, oxycodon, oxymorphon, pentazocin, asmazindol, pseudoephidrine, hydroxydion, propofol, acebutolol, albuterol, alprenolol, atenolol, betazolol, bucindolol, cartelolol, celiprolol, cetamolol, labetalol, levobunelol, metoprolol, metipranolol, nadolol, oxyprenolol, pindolol, propanolol, timolol, adrenalin, metaraminol, midodrin, norfenefrin, octapamine, oxedrin, oxilofrin, oximetazolin, phenylefrin, bamethan, clenbuterol, fenoterol, hexoprenalin, isoprenalin, isoxsuprin, orciprenalin, reproterol, salbutamol, terbutalin, carbuterol, dyphillin, etophyllin, fenoterol, pirbuterol, rimiterol, terbutalin, digitoxin, dobutamin, etilefrin, prenalterol, amphotericin B, chlorphenesin, nystatin, perimycin, acenocoumarol, dicoumarol, phenprocoumon, warfarin, bamethan, dipyrimadol, diprophyllin, isoxsuprin, vincamin, xantinol nicotinate, compactin, eptastatin, mevinolin, simvastatin, bromperidol, dithranol, ergotamine, ivermectin, metronidazole, secnizadole, nandrolon, propafenon, quinadine, quetiapine, serotonin, and silybin.

21. The composition of claim 14, wherein Y^{b} cleaves at a rate that provides therapeutic concentrations of the compound in said animal for a period of at least about 50% longer than the oral delivery of the compound itself using a similar oral dosage form.

22. The composition of claim 14, wherein Y^{b} cleaves at a rate that provides therapeutic concentrations of the compound in said animal for a period of at least about 100% longer than the oral delivery of the compound itself using a similar oral dosage form.

23. The composition of claim 14, including a pharmaceutically acceptable excipient selected from the group consisting of lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methyl cellulose.

24. The composition of claim 14, including a carrier selected from the group consisting of a tablet, pill, soft gelatin capsule, hard gelatin capsule, powder, lozenge, sachet, cachet, elixir, suspension, emulsion, solution and syrup.

25. The composition of claim 14, wherein the composition contains up to 90% by weight of the conjugate.


SEQUENCE LISTING (13 sequences)

SEQ ID NO: 1 - DNA, 1579 bp, Homo sapiens
aaagaaggcatccagcaagaactgcacaagaaacggagtcagccggagaacaaggagtggtcttccactgcctcacaggaggatggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatccaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagcccttcccctggtggcctggattctggtcccaaagcaattctgaaagccagtgtggtaaactagagagagcagcaaaaacaccagtcttgcctgagtctttctccagcatttccagtacatctatcagaatcatcaagtcttggccgggaacacagacagggtgtctacccaagaagcctcacctatccccaacttagaatttgctacttattttaaagacttgttcagtgactgtaaactctatgaaaccagaaaccgaatctgcctcttgctgggatctctaaaagtgtctgataagcatcttaaagtcactcaattcctgaactaatcaatatatatgtttaacccattactcaaatacccaaatcccattccaagttttgtgacccaaaagagaaataaatgctcacaagtgctgtagaattaaacttcagaagttctaacct

SEQ ID NO: 2 - DNA, 3779 bp, Homo sapiens
ttctattgaaagggaaatgggagaacaatatgtgttcctatggctcagtccctataagattctgtactattcagagttgattttaagtgtcacttaactgaaattatccaacaaaccttcatggcatgaaacattaacacagctctttttatatggcatggttcctatggctcaatccctataagattctgtactagttcagagttgattttaaaagtcacttaactgaaattatccaacaaaccctcgaggacattaaacattaacgtggctctttttatatggcatggttcattatcatgccaataaatgattaatcgtaactctctgtcttgaccaataattttgctggacttttgtgattcacaacgtgctctgtgttgtaatgctacctcttgaaactgacatcctagctttattgttttttattacttccctaaggtggctttcaaaagagacaccaagtgacatatttttaggaggggtttaaaagtttgatggggtagaagtaaacgttgcttaactcaaccagcagcagagccagggcccagggaccagcgcttctgtggacttggcctttccagcagcagacccagcaatgaatgatccgaacagctgtgtggacaatgcaacagtttgctctggtgcatcctgtgtggtacctgagagcaatttcaataacatcctaagtgtggtcctaagtacggtgctgaccatcctgttggccttggtgatgttctccatgggatgcaacgtggaaatcaagaaatttctagggcacataaagcggccgtggggcatttgtgttggcttcctctgtcagtttggaatcatgcccctcacaggattcatcctgtcggtggcctttgacatcctcccgctccaggccgtagtggtgctcattataggatgctgccctggaggaactgcctccaatatcttggcctattgggtcgatggcgacatggacctgagcgtcagcatgaccacatgctccacactgcttgccctcggaatgatgccgctgtgcctccttatctataccaaaatgtgggtcgactctgggagcatcgtaattccctatgataacataggtacatctctggttgctctcgttgttcctgtttccattggaatgtttgttaatcacaaatggccccaaaaagcaaagatcatacttaaaattgggtccatcgcgggcgccatcctcattgtgctcatagctgtggttggaggaatattgtaccaaagcgcctggatcattgctcccaaactgtggattataggaacaatatttcctgtggcgggttactccctggggtttcttctggctagaattgctggtctaccctggtacaggtgccgaacggttgcttttgaaacggggatgcagaacacgcagctatgttccaccatcgttcagctctccttcactcctgaggagctcaatgtcgtattcaccttcccgctcatctacagcattttccagctcgcctttgccgcaatattcttaggattttatgtggcatacaagaaatgtcatggaaaaaacaaggcagaaattccagagagcaaagaaaatggaacggagccagagtcatcgttttataaggcaaatggaggatttcaacctgacgaaaagtagacatcaagtggacaaaacagacgagttccaaattacgttcttaaaccgtaactatatttaattatttgttttggtaggacagttggcagaaaagagttaaagtgaaaattggaatttcattggaattcatgtattggtttcagtaccaagtgactggtggcccaattctttaatgggacaaatattgtttcctatatatatgtatatgttttatatatgtatgtatactcatatagatatattgtcattgaaatattcccccaaaatattctcagactaaacctgacatagggaacaccgagaatgaaaacatcgttaacaccaaaactgaattcttatgcagaatttcctagcccatagatgacaacctgagtttctgtatgttaaagtagatgtaatgaattattattattacagtggtcacgattttcttcagtgtttatgattataaaaattgacatgaacatctttcactgacattttaatcattattttaaaagctttgcaacctatatatttatataactttgtaatataacatgggcaaatatctgacttcagtatttttaaaaagttgccttctccagtggcagtccaaaagcagaaatgagaggaaattattacaaaatagaattcaataaccatattggatgcaggctcttaactcagcagggatatcgtacatctattgctctacctcaggggtccagtgatacccactagatcttccaaggaaaaacataattctttcaaacggtgtgtatttggcaaagagctcttcaaatctgggagagggacttcctcaaggttttcctgtgtgcagtggatccacatagctaatatgacagctagtcagttgacagggaccacccacagtaagcaccatggtcagggaggtggcaggaggtgcaaagacagaagtattgagagaaacaccaagactctagtggaggaattaattcaatgggagatagtataaaatacatagaaaacacaagtaacagaaacctggttgaaatgcttaactagagtcaattagatgtgcaggagtaagtagtataagaagaatcaagtccgagagtgatcaggaaatgagtattaaacagtatttgaaacagagaacgtgtcccagggcccaaaagtcagaagggccccaccagccaggaaagttgtttcaatgctgtaagtaggtgtagccaagggaagccaggactatctgatatacggtagcaggggtttacggctgccaggggaaaataactcatcaagtgttggactttcaattataagatcgaatttaatttcctttccctcattctgcagcaatcagaatacacaatcttaaccactcggtccttagtggttttgttccattttgcattgggtattttcactgcctcatagagtctatttcaagtgtttggctgaaagggctttttgcatttgcatgttctgagttcagattctgctggtgcacccaagcattatgggaacaggaactcaacttagctcttccagtagaggggtgagggattctgcttttcaaattcataacattgatctttttatgcaagatttccatttacagttgaataagtacttcatatttttccatcattagacaaatacaaaatggactaaataattttaagagatagtggaggcagcagggggtacagacttccttcttagagagtgtcagagaatatgctcccaatggtggaaaggaagatttacagtctagcggctaagtacctcctacacatttcccatcaatcagaaaatagacaggtacactaaagggacctgagaactcctcttgtaatttcaacacacccaaaatcaagggcctggatgccagcagctgcagcaagcaggtttttcctccctgttgagcaagacaggtgaggcaagataggacttggctttcttacatgatgcggtaacttgtgacttgagtctttttccctaatttgctagtgggaagaaaaatagctgagctttctaaaatgatagctctctatttttaaatgaatttgaaaagtcgattaaattatgtattttattgcctctgagtatcatattaaatgaatattttattttaaaggcttaaataaatgaaaatgatttttgt

SEQ ID NO: 3 - DNA, 1921 bp, Homo sapiens
ctgatttcaaaccactccttttcaaagatctctgagggagacattgcacctggccactgcagcccagagcaggtctggccacggccatgagcatgctgagccatcatgcccaccgtggatgacattctggagcaggttggggagtctggctggttccagaagcaagccttcctcatcttatgcctgctgtcggctgcctttgcgcccatctgtgtgggcatcgtcttcctgggtttcacacctgaccaccactgccagagtcctggggtggctgagctgagccagcgctgtggctggagccctgcggaggagctgaactatacagtgccaggcctggggcccgcgggcgaggccttccttggccagtgcaggcgctatgaagtggactggaaccagagcgccctcagctgtgtagaccccctggctagcctggccaccaacaggagccacctgccgctgggtccctgccaggatggctgggtgtatgacacgcccggctcttccatcgtcactgagttcaacctggtgtgtgctgactcctggaagctggacctctttcagtcctgtttgaatgcgggcttcttgtttggctctctcggtgttggctactttgcagacaggtttggccgtaagctgtgtctcctgggaactgtgctggtcaacgcggtgtcgggcgtgctcatggccttctcgcccaactacatgtccatgctgctcttccgcctgctgcagggcctggtcagcaagggcaactggatggctggctacaccctaatcacagaatttgttggctcgggctccagaagaacggtggcgatcatgtaccagatggccttcacggtggggctggtggcgcttaccgggctggcctacgccctgcctcactggcgctggctgcagctggcagtctccctgcccaccttcctcttcctgctctactactggtgtgtgccggagtcccctcggtggctgttatcacaaaaaagaaacactgaagcaataaagataatggaccacatcgctcaaaagaatgggaagttgcctcctgctgatttaaagatgctttccctcgaagaggatgtcaccgaaaagctgagcccttcatttgcagacctgttccgcacgccgcgcctgaggaagcgcaccttcatcctgatgtacctgtggttcacggactctgtgctctatcaggggctcatcctgcacatgggcgccaccagcgggaacctctacctggatttcctttactccgctctggtcgaaatcccgggggccttcatagccctcatcaccattgaccgcgtgggccgcatctaccccatggccatgtcaaatttgttggcgggggcagcctgcctcgtcatgatttttatctcacctgacctgcactggttaaacatcataatcatgtgtgttggccgaatgggaatcaccattgcaatacaaatgatctgcctggtgaatgctgagctgtaccccacattcgtcaggaacctcggagtgatggtgtgttcctccctgtgtgacataggtgggataatcacccccttcatagtcttcaggctgagggaggtctggcaagccttgcccctcattttgtttgcggtgttgggcctgcttgccgcgggagtgacgctacttcttccagagaccaagggggtcgctttgccagagaccatgaaggacgccgagaaccttgggagaaaagcaaagcccaaagaaaacacgatttaccttaaggtccaaacctcagaaccctcgggcacctgagagagatgttttgcggcgatgtcgtgttggagggatgaagatggagttatcctctgcagaaattcctagacgccttcacttctctgtattcttcctcatacttgcctacccccaaattaatatcagtcctaaagaaaaaaaaaaaaaaaaa

SEQ ID NO: 4 - DNA, 2214 bp, Homo sapiens
cctgtttcccaggaacggtccccggcttcgcgccccaatttctaacagcctgcctgtcccccgggaacgttctaacatccttggggagcgccccagctacaagacactgtcctgagaacgctgtcatcacccgtagttgcaagtttcggagcggcagtgggaagcatgcgggactacgacgaggtgatcgccttcctgggcgagtgggggcccttccagcgcctcatcttcttcctgctcagcgccagcatcatccccaatggcttcaatggtatgtcagtcgtgttcctggcggggaccccggagcaccgctgtcgagtgccggacgccgcgaacctgagcagcgcctggcgcaacaacagtgtcccgctgcggctgcgggacggccgcgaggtgccccacagctgcagccgctaccggctcgccaccatcgccaacttctcggcgctcgggctggagccggggcgcgacgtggacctggggcagctggagcaggagagctgcctggatggctgggagttcagccaggacgtctacctgtccaccgtcgtgaccgagtggaatctggtgtgtgaggacaactggaaggtgcccctcaccacctccctgttcttcgtaggcgtgctcctcggctccttcgtgtccgggcagctgtcagacaggtttggcaggaagaacgttctcttcgcaaccatggctgtacagactggcttcagcttcctgcagattttctccatcagctgggagatgttcactgtgttatttgtcatcgtgggcatgggccagatctccaactatgtggtagccttcatactaggaacagaaattcttggcaagtcagttcgtattatattctctacattaggagtgtgcacattttttgcagttggctatatgctgctgccactgtttgcttacttcatcagagactggcggatgctgctgctggcgctgacggtgccgggagtgctgtgtgtcccgctgtggtggttcattcctgaatctccccgatggctgatatcccagagaagatttagagaggctgaagatatcatccaaaaagctgcaaaaatgaacaacatagctgtaccagcagtgatatttgattctgtggaggagctaaatcccctgaagcagcagaaagctttcattctggacctgttcaggactcggaatattgccataatgaccattatgtctttgctgctatggatgctgacctcagtgggttactttgctctgtctctggatgctcctaatttacatggagatgcctacctgaactgtttcctctctgccttgattgaaattccagcttacattacagcctggctgctattgcgaaccctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcctcaccctttttttccctgaaagtttgggaatgactcttccagaaaccttagagcagatgcagaaagtgaaatggttcagatctgggaaaaaaacaagagactcaatggagacagaagaaaatcccaaggttctaataactgcattctgaaaaaatatctaccccatttggtgaagtgaaaaacagaaaaataagaccctgtggagaaattcgttgttcccactgaaatggactgactgtaacgattgacaccaaaatgaaccttgctatcaagaaatgctcgtcatacagtaaactctggatgattcttccagataatgtccttgctttacaaaccaaccatttctagagagtctccttactcattaattcaatgaaatggattggtaagatgtcttgaaaacatgttagtcaaggactggtaaaatacatataaagattaacactcatttccaatcatacaaatactatccaaataaaaataacatcattgtattaacgcaaatattaggtgacaacaaaaaaaaaaaaaaaaaaaaaaaa

SEQ ID NO: 5 - DNA, 3252 bp, Homo sapiens
gcggcccaggcccgcaaccttccctggtcgtgcgccctatgtaaggccagccgcggcaggaccaaggcggcggtgtcagctcgcgagcctaccctccgcggacggtcttgggtcgcctgctgcctggcttgcctggtcggcggcgggtgccccgcgcgcacgcgcaaagcccgccgcgttccccgaccccaggccgcgctctgtgggcctctgagggcggcatgcgggactacgacgaggtgaccgccttcctgggcgagtgggggcccttccagcgcctcatcttcttcctgctcagcgccagcatcatccccaatggcttcaccggcctgtcctccgtgttcctgatagcgaccccggagcaccgctgccgggtgccggacgccgcgaacctgagcagcgcctggcgcaaccacactgtcccactgcggctgcgggacggccgcgaggtgccccacagctgccgccgctaccggctcgccaccatcgccaacttctcggcgcttgggctggagccggggcgcgacgtggacctggggcagctggagcaggagagctgtctggatggctgggagttcagtcaggacgtctacctgtccaccattgtgaccgagtggaacctggtgtgtgaggacgactggaaggccccactcacaatctccttgttcttcgtgggtgtgctgttgggctccttcatttcagggcagctgtcagacaggtttggccggaagaatgtgctgttcgtgaccatgggcatgcagacaggcttcagcttcctgcagatcttctcgaagaattttgagatgtttgtcgtgctgtttgtccttgtaggcatgggccagatctccaactatgtggcagcatttgtcctggggacagaaattcttggcaagtcagttcgtataatattctctacgttaggagtgtgcatattttatgcatttggctacatggtgctgccactgtttgcttacttcatccgagactggcggatgctgctggtggcgctgacgatgccgggggtgctatgcgtggcactctggtggttcatccctgagtccccccgatggctcatctctcagggacgatttgaagaggcagaggtgatcatccgcaaggctgccaaagccaatgggattgttgtgccttccactatctttgacccgagtgagttacaagacctaagttccaagaagcagcagtcccacaacattctggatctgcttcgaacctggaatatccggatggtcaccatcatgtccataatgctgtggatgaccatatcagtgggctattttgggctttcgcttgatactcctaacttgcatggggacatctttgtgaactgcttcctttcagcgatggttgaagtcccagcatatgtgttggcctggctgctgctgcaatatttgccccggcgctattccatggccactgccctcttcctgggtggcagtgtccttctcttcatgcagctggtacccccagacttgtattatttggctacagtcctggtgatggtgggcaagtttggagtcacggctgccttttccatggtctacgtgtacacagccgagctgtatcccacagtggtgagaaacatgggtgtgggagtcagctccacagcatcccgcctgggcagcatcctgtctccctacttcgtttaccttggtgcctacgaccgcttcctgccctacattctcatgggaagtctgaccatcctgacagccatcctcaccttgtttctcccagagagcttcggtaccccactcccagacaccattgaccagatgctaagagtcaaaggaatgaaacacagaaaaactccaagtcacacaaggatgttaaaagatggtcaagaaaggcccacaatccttaaaagcacagccttctaacatcgcttccagtaagggagaaactgaagaggaaagactgtcttgccagaaatggccagcttgtgcagactccgagtccttcagtgacaaaggcctttgctgtttgtcctcttgacctgtgtctgacttgctcctggatgggcacccacactcagaggctacatatggccctagagcaccaccttcctctagggacactggggctacctacagacaacttcatctaagtcctaactattacaatgatggactcagcacctccaaagcagttaatttttcactagaaccagtgagatctggaggaatgtgagaagcatatgctaaatgtacattttaattttagactacttgaaaaggcccctaataaggctagaggtctaagtcccccacccctttccccactcccctctagtggtgaactttagaggaaaaggaagtaattgcacaaggagtttgattcttaccttttctcagttacagaggacattaactggatcattgcttccccagggcaggagagcgcagagctagggaaagtgaaaggtaatgaagatggagcagaatgagcagatgcagatcaccagcaaagtgcactgatgtgtgagctcttaagaccactcagcatgacgactgagtagacttgtttacatctgatcaaagcactgggcttgtccaggctcataataaatgctccattgaatctactattcttgttttccactgctgtggaaacctccttgctactatagcgtcttatgtatggtttaaaggaaatttatcaggtgagagagatgagcaacgttgtcttttctctcaaagctgtaatgtgggttttgttttattgtttatttgtttgttgttgtatccttttctccttgttatttgcccttcagaatgcacttgggaaaggctggttccttagcctcctggtttgtgtctttttttttttttttttaaacacagaatcactctggcaattgtctgcagctgccactggtgcaaggccttaccagccctagcctctagcacttctctaagtgccaaaaacagtgtcattgtgtgtgttcctttcttgatacttagtcatgggaggatattacaaaaaagaaatttaaattgtgttcatagtctttcagagtagctcactttagtcctgtaactttattgggtgatattttgtgttcagtgtaattgtcttctctttgctgattatgttaccatggtactcctaaagcatatgcctcacctggttaaaaaagaacaaacatgtttttgtgaaagctactgaagtgccttgggaaatgagaaagttttaataagtaaaatgattttttaaatatcaaaaaaaaaaaaaaaa

SEQ ID NO: 6 - DNA, 2553 bp, Homo sapiens
actcccacctccagagtccaagggtctatgtggtgggcagtttgagctggctggatactagagggaggctgcacctgaagcatttggtgggtgagcagcatgggctttgaggagctgctggagcaggtgggcggctttgggcccttccaactgcggaatgtggcactgctggccctgccccgagtgctgctaccactgcacttcctcctgcccatcttcctggctgccgtgcctgcccaccgatgtgccctgccgggtgcccctgccaacttcagccatcaggatgtgtggctggaggcccatcttccccgggagcctgatggcacgctcagctcctgcctccgctttgcctatccccaggctctccccaacaccacgttgggggaagaaaggcagagccgtggggagctggaggatgaacctgccacagtgccctgctctcagggctgggagtacgaccactcagaattctcctctaccattgcaactgagtgggatctggtgtgtgagcagaaaggtctgaacagagctgcgtccactttcttcttcgccggtgtgctggtgggggctgtggcctttggatatctgtccgacaggtttgggcggcggcgtctgctgctggtagcctacgtgagtaccctggtgctgggcctggcatctgcagcctccgtcagctatgtaatgtttgccatcacccgcacccttactggctcagccctggctggttttaccatcatcgtgatgccactggagctggagtggctggatgtggagcaccgcaccgtggctggagtcctgagcagcaccttctggacagggggcgtgatgctgctggcactggttgggtacctgatacgggactggcgatggcttctgctagctgtcaccctgccttgtgccccaggcatcctcagcctctggtgggtgcctgagtctgcacgctggcttctgacccaaggccatgtgaaagaggcccacaggtacttgctccactgtgccaggctcaatgggcggccagtgtgtgaggacagcttcagccaggaggctgtgagcaaagtggccgccggggaacgggtggtccgaagaccttcatacctagacctgttccgcacaccacggctccgacacatctcactgtgctgcgtggtggtgtggttcggagtgaacttctcctattacggcctgagtctggatgtgtcggggctggggctgaacgtgtaccagacacagctgttgttcggggctgtggaactgccctccaagctgctggtctacttgtcggtgcgctacgcaggacgccgcctcacgcaagccgggacactgctgggcacggccctggcgttcggcactagactgctagtgtcctccgatatgaagtcctggagcactgtcctggcagtgatggggaaagctttttctgaagctgccttcaccactgcctacctgttcacttcagagttgtaccctacggtgctcagacagacagggatggggctgactgcactggtgggccggctggggggctctttggccccactggcggccttgctggatggagtgtggctgtcactgcccaagcttacttatggggggatcgccctgctggctgccggcaccgccctcctgctgccagagacgaggcaggcacagctgccagagaccatccaggacgtggagagaaagagtgccccaaccagtcttcaggaggaagagatgcccatgaagcaggtccagaactaagtgggagtggaggcaggccctccacagaagctctgcagcaggggctgggagagcagaagggcaggccctgcaactcaggctgggagtatcgaaccctctgcctagggccggagttgctgccagtacccgctccctctgctcatccatccttgattatttggcttctaggaacagttgacttcccagaatgcagtgggctgctgggcacccctctcacggttggggaggattctgtaaataaaggtgccccttgggttggggcagtggtgacgagctgtgggaagagccctggataggaagccactgagtctgccctgggctctgataaaaccttcaccattaacttgctgtgtgaccttgggcatgtggctttccctctctgggcctcagtctgttcatctcccaaatggataatgaagcctcttgggaggccctaccataggatctgttgccatgctcaaatgagttactgaataaggtgcttctgcttcttctagagatggtgctaaagaaaggactagcatatgagacttctggtaccaatggggctggtgggcatgctgtccactgtgtggtgctaggactgccaatgccaggcccaagggacaaaaagaacagagctttttgttctcatggctggccctgctacctccgaggcaccctgcagggcaatgcatgtcatcccaacccccacactccccatcctccaacccactggtctcatgcccaaagaagagttgaaggcatgggagccaacattttattgaagaagccacagaggctgaaattcaataaacacaagttttatgagta

SEQ ID NO: 7 - DNA, 2712 bp, Homo sapiens
gagactttaacatcagaaaaaggatggacttgttgcagttgctgtagcattcaaagtcaaggtgatcatttcaaaccaagcatcagcaacaattaaaaatattcacttggtatctgtagtttaataatggaccaacatcaacatttgaataaaacagcagagtcagcatcttcagagaaaaagaaaacaagacgctgcaatggattcaagatgttcttggcagccctgtcattcagctatattgctaaagcactaggtggaatcattatgaaaatttccatcactcaaatagaaaggagatttgacatatcctcttctcttgctggtttaattgatggaagctttgaaattggaaatttgcttgtgattgtatttgtaagttactttggatctaaactacacagaccgaagttaattggaattggttgtctccttatgggaactggaagtattttgacatctttaccacatttcttcatgggatattataggtattctaaagaaacccatattaatccatcagaaaattcaacatcaagtttatcaacctgtttaattaatcaaaccttatcattcaatggaacatcacctgagatagtagaaaaagattgtgtaaaggaatctgggtcacacatgtggatctatgtcttcatggggaatatgcttcgtggcataggggaaacccccatagtaccattggggatttcatacattgatgattttgcaaaagaaggacattcttccttgtatttaggtagtttgaatgcaataggaatgattggtccagtcattggctttgcactgggatctctgtttgctaaaatgtacgtggatattggatatgtagatctgagcactatcagaataactcctaaggactctcgttgggttggagcttggtggcttggtttccttgtgtctggactattttccattatttcttccataccattttttttcttgccgaaaaatccaaataaaccacaaaaagaaagaaaaatttcactatcattgcatgtgctgaaaacaaatgatgatagaaatcaaacagctaatttgaccaaccaaggaaaaaatgttaccaaaaatgtgactggttttttccagtctttgaaaagcatccttaccaatcccctgtatgttatatttctgcttttgacattgttacaagtaagcagctttattggttcttttacttacgtctttaaatatatggagcaacagtacggtcagtctgcatctcatgctaactttttgttgggaatcataaccattcctacggttgcaactggaatgtttttaggaggatttatcattaaaaaattcaaattgtctttagttggaattgccaaattttcatttcttacttcgatgatatccttcttgtttcaacttctatatttccctctaatctgcgaaagcaaatcagttgccggcctaaccttgacctatgatggaaataattcagtggcatctcatgtagatgtaccactttcttattgcaactcagagtgcaattgtgatgaaagtcagtgggaaccagtctgtgggaacaatggaataacttacctgtcaccttgtctagcaggatgcaaatcctcaagtggtattaaaaagcatacagtgttttataactgtagttgtgtggaagtaactggtctccagaacagaaattactcagcacacttgggtgaatgcccaagagataatacttgtacaaggaaatttttcatctatgttgcaattcaagtcataaactctttgttctctgcaacaggaggtaccacatttatcttgttgactgtgaagattgttcaacctgaattgaaagcacttgcaatgggtttccagtcaatggttataagaacactaggaggaattctagctccaatatattttggggctctgattgataaaacatgtatgaagtggtccaccaacagctgtggagcacaaggagcttgtaggatatataattccgtattttttggaagggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaacattgcattgattcattaagatgttatttttgaggtgttcctggtctttcactgacaattccaacattctttacttacagtggaccaatggataagtctatgcatctataataaactataaaaaatgggagtacccatggttaggatatagctatgcctttatggttaagattagaatatatgatccataaaaatttaaagtgagaggcatggttagtgtgtgatacaataaaaagtaattgtttggtagttgtaactgctaataaaaccagtgactagaatataagggaggtaaaaaggacaagatagattaatagcctaaataaagagaaaagcctgatgcctttaaaaaaaatgaaacactttggatgtattacttaggccaaaatctggcctggatttatgctataatatatattttcatgttaagttgtatatttttcagaaattataaatattattaatttaaaatttgaa

SEQ ID NO: 8 - DNA, 2343 bp, Homo sapiens
gtgaagtatgtttttatgagcaaatttacctttctctccaagttctcccaaatttggaaaccctgagaaaccctgaaaatactctgtcctgggcttatatgccttagacacaactgggattgctgagctcaagggttgcagagcatcctactatgggaagaatgaggacataactcagattgttctggatatttccctcattgctgagaatgcagaatgtttctgaccaagaattgcaagcaagaacaggaaagagtggagtcggccaagatgtttctgttggcaataacatgtgcatttgtatccaaaacactgtctggatcttatatgaattccatgctcacacaaatagagagacaattcaacatcccaacatctctagttggattcattaatggaagctttgagattggaaatcttttgttgattatatttgtgagttattttggaaccaaactgcatagacctataatgattggcattggatgtgtggttatgggcttaggctgtttcttaaaatcactacctcatttcctcatgaaccaatatgaatatgaatctacagtttcagtttcaggcaacttgtcctcaaacagtttcttgtgtatggaaaatggaacccagattttaagaccaacgcaggatccatcagagtgtacaaaggaagttaaatcattaatgtgggtgtacgtcctagtaggcaatattgtacgtggaatgggtgaaactcccatcctgcctttgggtatttcctatatagaagattttgccaaatttgaaaattctcctttatatattgggcttgtagaaacaggagctattattggtcctttgattggacttttgttggcatcattctgtgcaaatgtttatgttgacactggatttgtgaacacagatgatctgatcataactcccactgacactcgttgggtcggtgcatggtggtttggctttctgatttgtgcaggagttaacgtgctcactgccattccttttttctttttgcccaacacacttccaaaggaaggactagagactaatgctgacatcattaaaaatgaaaatgaagacaaacaaaaagaagaggtcaagaaggaaaaatatggaatcactaaagattttctacctttcatgaaaagtctttcctgcaatccaatttatatgcttttcatacttgtaagtgtgatacagttcaatgcattcgttaacatgatctccttcatgcctaaatacctagaacagcaatatggaatatcatcttcagatgcaatctttctaatgggtatttataacttacctccaatatgtattggatatataattggtggtttaattatgaagaagttcaagattactgtcaaacaagctgcccacataggatgttggttatccttacttgagtatcttctctattttttatcttttctcatgacttgtgaaaattcttcagttgttggaataaatacctcttatgaaggaattccacaagatttatatgtggaaaatgacatctttgctgattgcaatgtggattgcaactgtccatctaaaatatgggatcctgtgtgtggaaacaatggcttgtcatatctgtcagcttgtcttgctggttgtgagacatccattggaacgggaataaacatggtgttccaaaattgcagctgtattcaaacatcaggaaattcatctgcagttcttgggctgtgcgacaaaggacctgactgttccttgatgctccagtacttcctaatcttgtcagcgatgagcagtttcatttattctttggctgccatacctggatatatggttctcttgaggtgtatgaaatctgaagagaagtcccttggtgtgggattacatacattttgcacaagagtatttggtaaaaatagttattttttcccacatcctatcagaacaatattattattattattttttattacactttaagttttagggtacatgtgcacaacgtgcaggtttgttacatatgtatacatgtgccatgttggtgtgctgcacccattaactcatcatttaacattaggtgaatctcctaatgctatccctccccccttcccccaccccacaacaggccctggtgtgtgatgttccccttcctgtgtccatgtgcagaacaatattaaatactagataaacaaggtaatattatttcagaaaagcagtctagcaaatgtcccttctgtatgtaaaaagcacgttataaatatgagaatgcagcacacaaagagatagagaatctcttttacttgaaaaggccctgtctgaccttaattacactattctttgtttgaagatttgaaaataaaaaaaaaa

SEQ ID NO: 9 - DNA, 4108 bp, Homo sapiens
gtgacccagggagacaaacacttggagatacttggggctgagtttgagcaagactccctaacctgtgtctggacaggtctgatgtcctgtgtggcccaagaagaactgaccccgtgtctggagctcccaccgttattgcatccctgctgtggctcacctgctgctgtctccaggagcccctgagaagatttgcttcctctcccctgctaagctccaggtcctgagattaaattaggggctggagctcactgcactccagcagtcatgggacccaggatagggccagcgggtgaggtaccccaggtaccagacaaggaaaccaaagccacaatgggcacagaaaacacacctggaggcaaagccagcccagaccctcaggacgtgcggccaagtgtgttccataacatcaagctgttcgttctgtgccacagcctgctgcagctggcgcagctcatgatctccggctacctaaagagctccatctccacagtggagaagcgcttcggcctctccagccagacgtcggggctgctggcctccttcaacgaggtggggaacacagccttgattgtgtttgtgagctattttggcagccgggtgcaccgaccccgaatgattggctatggggctatccttgtggccctggcgggcctgctcatgactctcccgcacttcatctcggagccataccgctacgacaacaccagccctgaggatatgccacaggacttcaaggcttccctgtgcctgcccacaacctcggccccagcctcggccccctccaatggcaactgctcaagctacacagaaacccagcatctgagtgtggtggggatcatgttcgtggcacagaccctgctgggcgtgggcggggtgcccattcagccctttggcatctcctacatcgatgactttgcccacaacagcaactcgcccctctacctcgggatcctgtttgcagtgaccatgatggggccaggcctggcctttgggctgggcagcctcatgctgcgcctttatgtggacattaaccagatgccagaaggtggtatcagcctgaccataaaggacccccgatgggtgggtgcctggtggctgggtttcctcatcgctgccggtgcagtggccctggctgccatcccctacttcttcttccccaaggaaatgcccaaggaaaaacgtgagcttcagtttcggcgaaaggtcttagcagtcacagactcacctgccaggaagggcaaggactctccctctaagcagagccctggggagtccacgaagaagcaggatggcctagtccagattgcaccaaacctgactgtgatccagttcattaaagtcttccccagggtgctgctgcagaccctacgccaccccatcttcctgctggtggtcctgtcccaggtatgcttgtcatccatggctgcgggcatggccaccttcctgcccaagttcctggagcgccagttttccatcacagcctcctacgccaacctgctcatcggctgcctctccttcccttcggtcatcgtgggcatcgtggtgggtggcgtcctggtcaagcggctccacctgggccctgtgggatgcggtgccctttgcctgctggggatgctgctgtgcctcttcttcagcctgccgctcttctttatcggctgctccagccaccagattgcgggcatcacacaccagaccagtgcccaccctgggctggagctgtctccaagctgcatggaggcctgctcctgcccattggacggctttaaccctgtctgcgaccccagcactcgtgtggaatacatcacaccctgccacgcaggctgctcaagctgggtggtccaggatgctctggacaacagccaggttttctacaccaactgcagctgcgtggtggagggcaaccccgtgctggcaggatcctgcgactcaacgtgcagccatctggtggtgcccttcctgctcctggtcagcctgggctcggccctggcctgtctcacccacacaccctccttcatgctcatcctaagaggagtgaagaaagaagacaagactttggctgtgggcatccagttcatgttcctgaggattttggcctggatgcccagccccgtgatccacggcagcgccatcgacaccacctgtgtgcactgggccctgagctgtgggcgtcgagctgtctgtcgctactacaataatgacctgctccgaaaccggttcatcggcctccagttcttcttcaaaacaggttctgtgatctgcttcgccttagttttggctgtcctgaggcagcaggacaaagaggcaaggaccaaagagagcagatccagccctgccgtagagcagcaattgctagtgtcggggccagggaagaagccagaggattcccgagtgtgagctgtcttggggccccacctggccaagagtagcagccacagcagtacctcctctgagtcctttgcccaagattgggtgtcaagagccctgtgttccattctggctcctccactaaattgctgtgtgacttcaggcaagacattgatcctctctcagcctttgcttgctagtctgaaccaaagagttgtttgggcatttgctgtgttggccatttctggagcaagagggtcttcttcctccttcccccagccagccagctgtcctggggccaggctttcctgggtggaaagaagtatacctttccctggggccctaggatagcaaagtgagccatagtgggccaggctgccctccatgctgggccccagcccaggtctgcactcgcctggatcaccttctttgagccttagccatctcctgtcaggtaggaatgaacttgccagccttcaggctcgttcagctatgaccatctgtgcggtcagggtacactcagctctcctccccaactccagcagcctttaagaagtgtccctttggcgccccctggaggcagagcactgagctggaccctgggtagactcccacagggaggacggagctggcctcaggagtgggacacccagacttggcagggccttcaagaggcctgtgtgggggccccaggaatccttagctgaagcggggagactcactctccatctcaggaaattctagcccttgccctcagggagccacggttgagggtgaggcccaacacctgccttagggccctgggtgggcaagtctgggccctggggtagggagggagactcaggcccacacttgggtattttctaatttcagacaaacacacactcagcgcgcactcactgattcctacacattgccaagatttcacacatgtgaccaggggccaccaaagtccctgtgacctttgtgactaggatcctaatttctctattttctcctgggtgcctgggtctgtgtcacctggggcagtgtggataatgtttagttctgtgacactgttttttgggggtggcacctggttctccgatgcctgggctggtgtcaggcccaggactgtagtgctgggagcagtaaagctcagctctgtgtaatgagtgatgctatggcttgctcgtgtcttatgatccaatccttttctacatcagcccttgttttgttttatggctagtcttatctggcctggttatttccttgcggggaggagagggtttgctaatctgctcccagcccaacctattaccaccccacctcgctgggacctactgctcgggaggcagcagacagggagccaccagcagtggcttcctggccctgtgctgggggtggggggaagctgggggcacatgtggcccttgccttctgagcagctcccagtgccagggctttgagactttcccacatgataaaagaaaagggaggtacagaagttccaattccctttttattttgctggttggtatctgtaaatgtttaataaatatctgagcatgtatctatcaacgccaagaatttcaaagtctccttcaacaatatgaggcttttaggatgtttatattccttcatccctcttgtttcccaggttttgcagggaaaaaaagtctggaattatagatacagcttattattaaatttgttcttgcataaaaaaaaaaaaaaaaaaaaaaaaaaaaaaaaaaaaaaaaaa

SEQ ID NO: 10 - DNA, 3927 bp, Homo sapiens
agagggcgcgcgcggctgaaagcgtgtggaggcgcgggctgcagttcggatgtctgtgtggcggggagggggcggcggccgggagagacgactccgccccctgcgcgcatgctccggccccggcgggttataaggcagcctcgctggcccggccagacaaagtggtgagctgcgacgtgactggctagctgcgtgggtactggaacaagcaaacgaggcagcgagcgaaggacgggagccggaccctgggccccgtggaactccagcctgcgccaccacgtcacgcacacgctcggcgctgcgatccgcgcatataacgatatttggatttgacctgcattttggaatttatctacacttaaaatgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaagcagcagaatctccggaccagaaagacacagatggagggcccaaggaggaggaaagtccagtctgaatccatggggctgaagggtaaattgagcagttcatgacccaggatatctgaaaatattctactggcctgtaatctaccagtggtgctcaatgcaaatagtagacatttgtgtggaaatcataccagttgttcattgatgggatttttgtttgactccttaccaatagcctgaatttgaggagggaatgattggtagcaaaggatgggggaaagaagtaggttctgttttgttttgttttaatcttagcttttaatagtgtcataaagattataatatgtgccttaagttttagtctttagaactctagagagccttaacttcttaaaccatttttgctgaattcatctatttcgagtgttgtgttaaaaggaaaaataacaactaacttgtttgaggcaaatctaaaatttaaaattaatcttgcttcattgttacatgtaatatatttcagacattttcactggaagatttatgaacagaaatattggttgaaagttagagattttacaaaatgctgacaaaaatattttcctagcatcagtagatttctggcatatgtttctgctagctatatatttaggaaattcaaagcataaaactttggcaacatcttggctgttctagacacagtgtacttgtcaacccctctcaggtaccttttcttgggatgcttattagaagccaagtaaagtgcttaaggtttgttttcattaaattagctatttctgctcccctgttcaaagatgcattttgagtgtttatagatcactgccctttttgaaatcacctggtattatttttcttactggaaaagttagtattaaaatctacagaactacatatttgtgcctccttggtaaatacaacacatctaattaaatgtagacagatatttcaaacatcagctgaattcacttaagtttttccaaaacctcagttaaactgtgaagctattggaatttttttttcctggaatttttcccctttgattcacagtggtcccatttatatctgcttctagcttagtgctatgtgtgagatatgtgtgtgtttggtgtttttgtttttttgtttttttttttttaaggtttgcaaattaaaaagggccagaaaaatttggcaccaggcaaacgaataaagataggattgggaaagaagttgctaagtgtgcttagttttaataagtaattccttctcttttttcagagaaggccttacagaaaattgttgtgcttagaattgctggatgcatttttaccctccacacaaacctaaaaattttgtgacccctttcacttacctgaaaagtagagaaatggattcagtataaggataaggagggaaggtggaccagaatgaaaactgtaaatatttttttaacctaatatcacttaaatcgaggcagaaagatacagacattcaatgaattatattcaatgcatttaaaataccactgtaattgacagagtaaaagtatagatacaaaaccttgtgtaagaggctgacttttccaaataaacattttttaagaaaacatttcttctcccaaatgtctattttcttgaggaaaatattgctgtgtcttcattttcattaccaggtttcattttgggccttgctaaattgattgaattaaatcctccagcttttgaaccttgatatttgtgtatatgatttattttcatttgaatttctcctttcctcttctttgctgtaaggcaaggaggaggggaattttaaaaccatcttatttgaactgagagcatccagagcagttaaccttaaggaaacaatgaaaaactccctttgtatgcctgggcatcatggcagatagaggaagagtgttagaggagaaaactgctgctgagagtattggcaggcttggcctcagtttggactctgtaattttctttggacccaagtctgtaacctctgcgtacttcttctctcttaccttctataaaaatgaggattactgttggtgagggaataaggaatgtaagtaaaggaaatctgaaaaaataaaagtaaagcaagtataaaaaaaaaaaaaaaaaaaa

SEQ ID NO: 11 - DNA, 3045 bp, Homo sapiens
gaatgtccaaatcacacagtttctttggttatcccctgagcatcttcttcatcgtggtcaatgagttttgcgaaagattttcctactatggaatgcgagcaatcctgattctgtacttcacaaatttcatcagctgggatgataacctgtccaccgccatctaccatacgtttgtggctctgtgctacctgacgccaattctcggagctcttatcgccgactcgtggctgggaaagttcaagaccattgtgtcgctctccattgtctacacaattggacaagcagtcacctcagtaagctccattaatgacctcacagaccacaaccatgatggcacccccgacagccttcctgtgcacgtggtgctgtccttgatcggcctggccctgatagctctcgggactggaggaatcaaaccctgtgtgtctgcgtttggtggagatcagtttgaagagggccaggagaaacaaagaaacagatttttttccatcttttacttggctattaatgctggaagtttgctttccacaatcatcacacccatgctcagagttcaacaatgtggaattcacagtaaacaagcttgttacccactggcctttggggttcctgctgctctcatggctgtagccctgattgtgtttgtccttggcagtgggatgtacaagaagttcaagccacagggcaacatcatgggtaaagtggccaagtgcatcggttttgccatcaaaaatagatttaggcatcggagtaaggcatttcccaagagggagcactggctggactgggctaaagagaaatacgatgagcggctcatctcccaaattaagatggttacgagggtgatgttcctgtatattccactcccaatgttctgggccttgtttgaccagcagggctccaggtggacactgcaggcaacaactatgtccgggaaaatcggagctcttgaaattcagcccgatcagatgcagaccgtgaacgccatcctgatcgtgatcatggtcccgatcttcgatgctgtgctgtaccctctcattgcaaaatgtggcttcaatttcacctccttgaagaagatggcagttggcatggtcctggcctccatggcctttgtggtggctgccatcgtgcaggtggaaatcgataaaactcttccagtcttccccaaaggaaacgaagtccaaattaaagttttgaatataggaaacaataccatgaatatatctcttcctggagagatggtgacacttggcccaatgtctcaaacaaatgcatttatgacttttgatgtaaacaaactgacaaggataaacatttcttctcctggatcaccagtcactgctgtaactgacgacttcaagcagggccaacgccacacgcttctagtgtgggcccccaatcactaccaggtggtaaaggatggtcttaaccagaagccagaaaaaggggaaaatggaatcagatttgtaaatacttttaacgagctcatcaccatcacaatgagtgggaaagtttatgcaaacatcagcagctacaatgccagcacataccagttttttccttctggcataaaaggcttcacaataagctcaacagagattccgccacaatgtcaacctaatttcaatactttctaccttgaatttggtagtgcttatacctatatagtccaaaggaagaatgacagctgccctgaagtgaaggtgtttgaagatatttcagccaacacagttaacatggctctgcaaatcccgcagtattttcttctcacctgtggcgaagtggtcttctctgtcacgggattggaattctcatattctcaggctccttccaacatgaagtcggtgcttcaggcaggatggctgctgaccgtggctgttggcaacatcattgtgctcatcgtggcaggggcaggccagttcagcaaacagtgggccgagtacattctatttgccgcgttgcttctggtcgtctgtgtaatttttgccatcatggctcggttctatacttacatcaacccagcggagatcgaagctcaatttgatgaggatgaaaagaaaaacagactggaaaagagtaacccatatttcatgtcaggggccaattcacagaaacagatgtgaaggtcaggaggcaagtggaggatggactgggcccgcagatgccctgacctctgcccccaggtagcaggacactccattggatggcccctgatgaggaagacttcagaattgggaactaaaccatgaatgctattttcttttttctttttcttttctttttttttttttttttttttttgagacagagttttgctcttgttgtccaggctggagtgcaatggcacgatctcagctcactgcaacctccgcctcccaggttcaagtaattctcctgcctcagcctcccgagtggctgggattagcggcatgcaccaccacgcccagctatttttgtatttttagtagagatggggtttcaccatgttggccaggatggtctcgatctcttgacctggtgatctgcccacctcggcctgccaaagtgctgggattacaggcttgagctaccgcgcccggccgtgaacgctattttctaagcagccagcagtgaatctaaaactctggaagaagtcttctgtttgaaaggcttatttaagccacacgtacacacactgtcttagagtactgtgagcccaccccacattggtcatcttccctatcacacaaatgatgttattttggactagcttaattttgaaatggtaacaaagtttcctattccatactgttcatttctaatactcttacgaaaactattctaaaggaggcaggagccaaggccaaaagtgaacgtacaggtttgaaatggctgtgataaggaccagctggtattaactgataactttacctttgggtttttgttattttgtttttctagtccctacctgtgtttaaattatggataactcgaaagacagctcaggtgaaggccagtaatgatttttttgaagtttcaatggtgtgaaataaatttctgttctta

SEQ ID NO: 12 - DNA, 3162 bp, Homo sapiens
gcgccctagccctctttcggggatactggccgaccccctcttccttttcccctttagtgaaggcctcccccgtcgccgcgcggcttcccggagccgactgcagactccctcagcccggtgttccccgcgtccggacgccgaggtcgcggcttcgcagaaactcgggcccctccatccgccctcagaaaagggagcgatgttgatctcaggaagcacaaagggaccttcctagctctgactgaaccacggagctcaccctggacagtatcactccgtggaggaagactgtgagactgtggctggaagccagattgtagccacacatccgcccctgccctaccccagagccctggagcagcaactggctgcagatcacagacacagtgaggatatgagtgtaggggtgagcacctcagcccctctttccccaacctcgggcacaagcgtgggcatgtctaccttctccatcatggactatgtggtgttcgtcctgctgctggttctctctcttgccattgggctctaccatgcttgtcgtggctggggccggcatactgttggtgagctgctgatggcggaccgcaaaatgggctgccttccggtggcactgtccctgctggccaccttccagtcagccgtggccatcctgggtgtgccgtcagagatctaccgatttgggacccaatattggttcctgggctgctgctactttctggggctgctgatacctgcacacatcttcatccccgttttctaccgcctgcatctcaccagtgcctatgagtacctggagcttcgattcaataaaactgtgcgagtgtgtggaactgtgaccttcatctttcagatggtgatctacatgggagttgtgctctatgctccgtcattggctctcaatgcagtgactggctttgatctgtggctgtccgtgctggccctgggcattgtctgtaccgtctatacagctctgggtgggctgaaggccgtcatctggacagatgtgttccagacactggtcatgttcctcgggcagctggcagttatcatcgtggggtcagccaaggtgggcggcttggggcgtgtgtgggccgtggcttcccagcacggccgcatctctgggtttgagctggatccagacccctttgtgcggcacaccttctggaccttggccttcgggggtgtcttcatgatgctctccttatacggggtgaaccaggctcaggtgcagcggtacctcagttcccgcacggagaaggctgctgtgctctcctgttatgcagtgttccccttccagcaggtgtccctctgcgtgggctgcctcattggcctggtcatgttcgcgtattaccaggagtatcccatgagcattcagcaggctcaggcagccccagaccagttcgtcctgtactttgtgatggatctcctgaagggcctgccaggcctgccagggctcttcattgcctgcctcttcagcggctctctcagcactatatcctctgcttttaattcattggcaactgttacgatggaagacctgattcgaccttggttccctgagttctctgaagcccgggccatcatgctttccagaggccttgcctttggctatgggctgctttgtctaggaatggcctatatttcctcccagatgggacctgtgctgcaggcagcaatcagcatctttggcatggttgggggaccgctgctgggactcttctgccttggaatgttctttccatgtgctaaccctcctggtgctgttgtgggcctgttggctgggctcgtcatggccttctggattggcatcgggagcatcgtgaccagcatgggcttcagcatgccaccctctccctctaatgggtccagcttctccctgcccaccaatctaaccgttgccactgtgaccacactgatgcccttgactaccttctccaagcccacagggctgcagcggttctattccttgtcttacttatggtacagtgctcacaactccaccacagtgattgtggtgggcctgattgtcagtctactcactgggagaatgcgaggccggtccctgaaccctgcaaccatttacccagtgttgccaaagctcctgtccctccttccgttgtcctgtcagaagcggctccactgcaggagctacggccaggaccacctcgacactggcctgtttcctgagaagccgaggaatggtgtgctgggggacagcagagacaaggaggccatggccctggatggcacagcctatcaggggagcagctccacctgcatcctccaggagacctccctgtgatgttgactcaggaccccgcctctgtcctcactgtgccaggccatagccagaggccaccctgtagtacagggatgagtcttggtgtgttctgcagggacaggcctggatgatctagctcataccaaaggaccttgttctgagaggttcttgcctgcaggagaagctgtcacatctcaagcatgtgaggcaccgtttttctcgtcgcttgccaatctgttttttaaaggatcaggctcgtagggagcaggatcatgccagaaatagggatggaagtgcatcctctgggaaaaagataatggcttctgattcaacatagccatagtcctttgaagtaagtggctagaaacagcactctggttataattgccccagggcctgattcaggactgactctccaccataaaactggaagctgcttcccctgtagtccccatttcagtaccagttctgccagccacagtgagcccctattattactttcagattgtctgtgacactcaagcccctctcatttttatctgtctacctccattctgaagagggaggttttggtgtccctggtcctctgggaatagaagatccatttgtctttgtgtagagcaagcacgttttccacctcactgtctccatcctccacctctgagatggacacttaagagacggggcaaatgtggatccaagaaaccagggccatgaccaggtccactgtggagcagccatctatctacctgactcctgagccaggctgccgtggtgtcatttctgtcatccgtgctctgtttccttttggagtttcttctccacattatctttgttcctggggaataaaaactaccattggacctaaaaaaaaaaaaaaaaaa

SEQ ID NO: 13 - DNA, 5176 bp, Homo sapiens
ctccggcgcccgctctgcccgccgctgggtccgaccgcgctcgccttccttgcagccgcgcctcggccccatggacgccctgtgcggttccggggagctcggctccaagttctgggactccaacctgtctgtgcacacagaaaacccggacctcactccctgcttccagaactccctgctggcctgggtgccctgcatctacctgtgggtcgccctgccctgctacttgctctacctgcggcaccattgtcgtggctacatcatcctctcccacctgtccaagctcaagatggtcctgggtgtcctgctgtggtgcgtctcctgggcggaccttttttactccttccatggcctggtccatggccgggcccctgcccctgttttctttgtcacccccttggtggtgggggtcaccatgctgctggccaccctgctgatacagtatgagcggctgcagggcgtacagtcttcgggggtcctcattatcttctggttcctgtgtgtggtctgcgccatcgtcccattccgctccaagatccttttagccaaggcagagggtgagatctcagaccccttccgcttcaccaccttctacatccactttgccctggtactctctgccctcatcttggcctgcttcagggagaaacctccatttttctccgcaaagaatgtcgaccctaacccctaccctgagaccagcgctggctttctctcccgcctgtttttctggtggttcacaaagatggccatctatggctaccggcatcccctggaggagaaggacctctggtccctaaaggaagaggacagatcccagatggtggtgcagcagctgctggaggcatggaggaagcaggaaaagcagacggcacgacacaaggcttcagcagcacctgggaaaaatgcctccggcgaggacgaggtgctgctgggtgcccggcccaggccccggaagccctccttcctgaaggccctgctggccaccttcggctccagcttcctcatcagtgcctgcttcaagcttatccaggacctgctctccttcatcaatccacagctgctcagcatcctgatcaggtttatctccaaccccatggccccctcctggtggggcttcctggtggctgggctgatgttcctgtgctccatgatgcagtcgctgatcttacaacactattaccactacatctttgtgactggggtgaagtttcgtactgggatcatgggtgtcatctacaggaaggctctggttatcaccaactcagtcaaacgtgcgtccactgtgggggaaattgtcaacctcatgtcagtggatgcccagcgcttcatggaccttgcccccttcctcaatctgctgtggtcagcacccctgcagatcatcctggcgatctacttcctctggcagaacctaggtccctctgtcctggctggagtcgctttcatggtcttgctgattccactcaacggagctgtggccgtgaagatgcgcgccttccaggtaaagcaaatgaaattgaaggactcgcgcatcaagctgatgagtgagatcctgaacggcatcaaggtgctgaagctgtacgcctgggagcccagcttcctgaagcaggtggagggcatcaggcagggtgagctccagctgctgcgcacggcggcctacctccacaccacaaccaccttcacctggatgtgcagccccttcctggtgaccctgatcaccctctgggtgtacgtgtacgtggacccaaacaatgtgctggacgccgagaaggcctttgtgtctgtgtccttgtttaatatcttaagacttcccctcaacatgctgccccagttaatcagcaacctgactcaggccagtgtgtctctgaaacggatccagcaattcctgagccaagaggaacttgacccccagagtgtggaaagaaagaccatctccccaggctatgccatcaccatacacagtggcaccttcacctgggcccaggacctgccccccactctgcacagcctagacatccaggtcccgaaaggggcactggtggccgtggtggggcctgtgggctgtgggaagtcctccctggtgtctgccctgctgggagagatggagaagctagaaggcaaagtgcacatgaagggctccgtggcctatgtgccccagcaggcatggatccagaactgcactcttcaggaaaacgtgcttttcggcaaagccctgaaccccaagcgctaccagcagactctggaggcctgtgccttgctagctgacctggagatgctgcctggtggggatcagacagagattggagagaagggcattaacctgtctgggggccagcggcagcgggtcagtctggctcgagctgtttacagtgatgccgatattttcttgctggatgacccactgtccgcggtggactctcatgtggccaagcacatctttgaccacgtcatcgggccagaaggcgtgctggcaggcaagacgcgagtgctggtgacgcacggcattagcttcctgccccagacagacttcatcattgtgctagctgatggacaggtgtctgagatgggcccgtacccagccctgctgcagcgcaacggctcctttgccaactttctctgcaactatgcccccgatgaggaccaagggcacctggaggacagctggaccgcgttggaaggtgcagaggataaggaggcactgctgattgaagacacactcagcaaccacacggatctgacagacaatgatccagtcacctatgtggtccagaagcagtttatgagacagctgagtgccctgtcctcagatggggagggacagggtcggcctgtaccccggaggcacctgggtccatcagagaaggtgcaggtgacagaggcgaaggcagatggggcactgacccaggaggagaaagcagccattggcactgtggagctcagtgtgttctgggattatgccaaggccgtggggctctgtaccacgctggccatctgtctcctgtatgtgggtcaaagtgcggctgccattggagccaatgtgtggctcagtgcctggacaaatgatgccatggcagacagtagacagaacaacacttccctgaggctgggcgtctatgctgctttaggaattctgcaagggttcttggtgatgctggcagccatggccatggcagcgggtggcatccaggctgcccgtgtgttgcaccaggcactgctgcacaacaagatacgctcgccacagtccttctttgacaccacaccatcaggccgcatcctgaactgcttctccaaggacatctatgtcgttgatgaggttctggcccctgtcatcctcatgctgctcaattccttcttcaacgccatctccactcttgtggtcatcatggccagcacgccgctcttcactgtggtcatcctgcccctggctgtgctctacaccttagtgcagcgcttctatgcagccacatcacggcaactgaagcggctggaatcagtcagccgctcacctatctactcccacttttcggagacagtgactggtgccagtgtcatccgggcctacaaccgcagccgggattttgagatcatcagtgatactaaggtggatgccaaccagagaagctgctacccctacatcatctccaaccggtggctgagcatcggagtggagttcgtggggaactgcgtggtgctctttgctgcactatttgccgtcatcgggaggagcagcctgaacccggggctggtgggcctttctgtgtcctactccttgcaggtgacatttgctctgaactggatgatacgaatgatgtcagatttggaatctaacatcgtggctgtggagagggtcaaggagtactccaagacagagacagaggcgccctgggtggtggaaggcagccgccctcccgaaggttggcccccacgtggggaggtggagttccggaattattctgtgcgctaccggccgggcctagacctggtgctgagagacctgagtctgcatgtgcacggtggcgagaaggtggggatcgtgggccgcactggggctggcaagtcttccatgaccctttgcctgttccgcatcctggaggcggcaaagggtgaaatccgcattgatggcctcaatgtggcagacatcggcctccatgacctgcgctctcagctgaccatcatcccgcaggaccccatcctgttctcggggaccctgcgcatgaacctggaccccttcggcagctactcagaggaggacatttggtgggctttggagctgtcccacctgcacacgtttgtgagctcccagccggcaggcctggacttccagtgctcagagggcggggagaatctcagcgtgggccagaggcagctcgtgtgcctggcccgagccctgctccgcaagagccgcatcctggttttagacgaggccacagctgccatcgacctggagactgacaacctcatccaggctaccatccgcacccagtttgatacctgcactgtcctgaccatcgcacaccggcttaacactatcatggactacaccagggtcctggtcctggacaaaggagtagtagctgaatttgattctccagccaacctcattgcagctagaggcatcttctacgggatggccagagatgctggacttgcctaaaatatattcctgagatttcctcctggcctttcctggttttcatcaggaaggaaatgacaccaaatatgtccgcagaatggacttgatagcaaacactgggggcaccttaagattttgcacctgtaaagtgccttacagggtaactgtgctgaatgctttagatgaggaaatgatccccaagtggtgaatgacacgcctaaggtcacagctagtttgagccagttagactagtcccccggtctcccgattcccaactgagtgttatttgcacactgcactgttttcaaataacgattttatgaaatgacctctgtcctccctctgatttttcatattttcctaaagtttcgtttctgttttttaataaaaagctttttcctcctggaacagaagacagctgctgggtcaggccacccctaggaactcagtcctgtactctggggtgctgcctgaatccattaaaaatgggagtactgatgaaataaaactacatggtcaacagtaaaaaaaaaaaaaaaaaaa